SEQUENCE LISTING (61 sequences)

SEQ ID NO: 1 - protein, 54 aa, Artificial Sequence
  Description of Artificial Sequence note = synthetic construct
LVPHLGDREKRDSVCPQGKYIHPQNNSICCTKCHKGTYLYNDCPGPGQDTDCRE

SEQ ID NO: 2 - protein, 45 aa, Artificial Sequence
  Description of Artificial Sequence note = synthetic construct
YAPEPGSTCRLREYYDQTAQMCCSKCSPGQHAKVFCTKTSDTVCD

SEQ ID NO: 3 - protein, 43 aa, Artificial Sequence
  Description of Artificial Sequence note = synthetic construct
RLSSKSVNAQVTDINSKGLELRKTVTTVETQNLEGLHHDGQFC

SEQ ID NO: 4 - protein, 66 aa, Artificial Sequence
  Description of Artificial Sequence note = synthetic construct
RLSSKSVNAQVTDINSKGLELRKTVTTVETQNLEGLHHDGQFCHKPCPPGERKARDCTVNGDEPDC

SEQ ID NO: 5 - protein, 38 aa, Artificial Sequence
  Description of Artificial Sequence note = synthetic construct
CRDQEKEYYEPQHRICCSRCPPGTYVSAKCSRIRDTVC

SEQ ID NO: 6 - protein, 34 aa, Artificial Sequence
  Description of Artificial Sequence note = synthetic construct
CREKQYLINSQCCSLCQPGQKLVSDCTEFTETEC

SEQ ID NO: 7 - protein, 41 aa, Artificial Sequence
  Description of Artificial Sequence note = synthetic construct
CHGNPSHYYDKAVRRCCYRCPMGLFPTQQCPQRPTDCRKQC

SEQ ID NO: 8 - protein, 36 aa, Artificial Sequence
  Description of Artificial Sequence note = synthetic construct
CPERHYWAQGKLCCQMCEPGTFLVKDCDQHRKAAQC

SEQ ID NO: 9 - protein, 32 aa, Artificial Sequence
  Description of Artificial Sequence note = synthetic construct
CKEDEYPVGSECCPKCSPGYRVKEACGELTGT

SEQ ID NO: 10 - protein, 34 aa, Artificial Sequence
  Description of Artificial Sequence note = synthetic construct
CVGDTYPSNDRCCHECRPGNGMVSRCSRSQNTVC

SEQ ID NO: 11 - protein, 30 aa, Artificial Sequence
  Description of Artificial Sequence note = synthetic construct
CNRCTEGVGYTNASNNLFACLPCTACKSDE

SEQ ID NO: 12 - protein, 455 aa, Artificial Sequence
  Description of Artificial Sequence note = synthetic construct
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 13 - protein, 461 aa, Artificial Sequence
  Description of Artificial Sequence note = synthetic construct
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 14 - protein, 335 aa, Artificial Sequence
  Description of Artificial Sequence note = synthetic construct
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 15 - protein, 435 aa, Artificial Sequence
  Description of Artificial Sequence note = synthetic construct
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 16 - protein, 277 aa, Artificial Sequence
  Description of Artificial Sequence note = synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 17 - protein, 595 aa, Artificial Sequence
  Description of Artificial Sequence note = synthetic construct
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 18 - protein, 260 aa, Artificial Sequence
  Description of Artificial Sequence note = synthetic construct
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 19 - protein, 283 aa, Artificial Sequence [sequence omitted]

SEQ ID NO: 20 - protein, 277 aa, Artificial Sequence
  Description of Artificial Sequence note = synthetic construct
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 21 - protein, 468 aa, Artificial Sequence
  Description of Artificial Sequence note = synthetic construct
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSERPGACNRCTEGVGYTNASNNLFACLPCTACKSDEEERSPCTTTRNTACQCKPGTFRNDNSAEMCRKCSTGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAKEKIQDLLVDSGKFIYLEDGTGSAVSLE

SEQ ID NO: 22 - protein, 38 aa, Artificial Sequence
  Description of Artificial Sequence note = synthetic construct
CPQGKYIHPQNNSICCTKCHKGTYLYNDCPGPGQDTDC

SEQ ID NO: 23 - protein, 36 aa, Artificial Sequence
  Description of Artificial Sequence note = synthetic construct
CRLREYYDQTAQMCCSKCSPGQHAKVFCTKTSDTVC

SEQ ID NO: 24 - protein, 38 aa, Artificial Sequence; identical to SEQ ID NO: 5
  Description of Artificial Sequence note = synthetic construct

SEQ ID NO: 25 - protein, 34 aa, Artificial Sequence; identical to SEQ ID NO: 6
  Description of Artificial Sequence note = synthetic construct

SEQ ID NO: 26 - protein, 34 aa, Artificial Sequence
  Description of Artificial Sequence note = synthetic construct
CKEDEYPVGSECCPKCSPGYRVKEACGELTGTVC

SEQ ID NO: 27 - protein, 41 aa, Artificial Sequence; identical to SEQ ID NO: 7
  Description of Artificial Sequence note = synthetic construct

SEQ ID NO: 28 - protein, 42 aa, Artificial Sequence
  Description of Artificial Sequence note = synthetic construct
PYGADRGKCRGNDYEKDGLCCTSCPPGSYASRLCGPGSDTVC

SEQ ID NO: 29 - protein, 28 aa, Artificial Sequence
  Description of Artificial Sequence note = synthetic construct
EKDGLCCASCHPGFYASRLCGPGSNTVC

SEQ ID NO: 30 - protein, 22 aa, Artificial Sequence
  Description of Artificial Sequence note = synthetic construct
CTPCPNGTYVSGLYNCTDCTEC

SEQ ID NO: 31 - protein, 44 aa, Artificial Sequence
  Description of Artificial Sequence note = synthetic construct
HAPVNGSCDDGEYLDKTHNQCCNRCPPGEFAKIRCSGSDNTKCE

SEQ ID NO: 32 - protein, 44 aa, Artificial Sequence
  Description of Artificial Sequence note = synthetic construct
HAPVNGSCDEGEYLDKRHNQCCNRCPPGEFAKVRCNGNDNTKCE

SEQ ID NO: 33 - protein, 37 aa, Artificial Sequence
  Description of Artificial Sequence note = synthetic construct
CNGTDYNSNSNNLCCKQCDPGMYMTHSCNTTSNTKCD

SEQ ID NO: 34 - protein, 31 aa, Artificial Sequence
  Description of Artificial Sequence note = synthetic construct
YYNSQELKCCKLCKPGTYSDHRCDKYSDTIC

SEQ ID NO: 35 - protein, 38 aa, Artificial Sequence
  Description of Artificial Sequence note = synthetic construct
CRQGYYYDPESEMCFPCSNCESSKVKVTTCNRTHDTVC

SEQ ID NO: 36 - protein, 42 aa, Artificial Sequence
  Description of Artificial Sequence note = synthetic construct
YTPINGKCNGTDYNSNNLCCKQCNPGMYMTHSCNTTSNTKCD

SEQ ID NO: 37 - protein, 41 aa, Artificial Sequence
  Description of Artificial Sequence note = synthetic construct
YAPSNGKCKDNEYKRHNLCCLSCPPGTYASRLCDSKTNTQC

SEQ ID NO: 38 - protein, 40 aa, Artificial Sequence
  Description of Artificial Sequence note = synthetic construct
HAPSNGKCKDNEYRSRNLCCLSCPPGTYASRLCDSKTNTQ

SEQ ID NO: 39 - protein, 41 aa, Artificial Sequence
  Description of Artificial Sequence note = synthetic construct
YTPPNGKCKDTEYKRHNLCCLSCPPGTYASRLCDSKTNTQC

SEQ ID NO: 40 - protein, 124 aa, Artificial Sequence
  Description of Artificial Sequence note = synthetic construct
VCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVC

SEQ ID NO: 41 - protein, 33 aa, Artificial Sequence
  Description of Artificial Sequence note = synthetic construct
DINSEGMEDLSFDDDAQDDNANKTLETQNLEHD

SEQ ID NO: 42 - protein, 17 aa, Artificial Sequence
  Description of Artificial Sequence note = synthetic construct
TEINELRQTITDVETNL

SEQ ID NO: 43 - protein, 30 aa, Artificial Sequence
  Description of Artificial Sequence note = synthetic construct
DENSKELERLQKIAFNVETKTLESLVDEGQ

SEQ ID NO: 44 - protein, 326 aa, Artificial Sequence
  Description of Artificial Sequence note = synthetic construct
MFRLTLLLAYVACVYGGGAPYGADRGKCRGNDYEKDGLCCTSCPPGSYASRLCGPGSDTVCSPCKNETFTASTNHAPACVSCRGRCTGHLSESQSCDKTRDRVCDCSAGNYCLLKGQEGCRICAPKTKCPAGYGVSGHTRTGDVLCTKCPRYTYSDAVSSTETCTSSFNYISVEFNLYPVNDTSCTTTAGPNEVVKTSEFSVTLNHTDCDPVFHTEYYGTSGSEGAGGFFTGMDRYQNTTKMCTLNIEIRCVEGDAVRTIPRTSDGVGVLSHSETITVIGGCLSDVNVDIEYSDSNHPEEVDDFVEYHWGTRLRLFPSPKRCRLVS

SEQ ID NO: 45 - protein, 325 aa, Artificial Sequence
  Description of Artificial Sequence note = synthetic construct
MLRLIALLVCVVYVYGDDVPYSSNQGKCGGHDYEKDGLCCASCHPGFYASRLCGPGSNTVCSPCEDGTFTASTNHAPACVSCRGPCTGHLSESQPCDRTHDRVCNCSTGNYCLLKGQNGCRICAPQTKCPAGYGVSGHTRAGDTLCEKCPPHTYSDSLSPTERCGTSFNYISVGFNLYPVNETSCTTTAGHNEVIKTKEFTVTLNYTDCDPVFHTEYYATSGKEGAGGFFTGTDIYQNTTKVCTLNVEIQCSEGDDIHTLQKTNGGSTMPHSETITVVGSCLSDVNVDIMYSDTNHPGEVDDFVEYHWGTRLRFFPLPKRCTPVS

SEQ ID NO: 46 - protein, 175 aa, Artificial Sequence
  Description of Artificial Sequence note = synthetic construct
MKPLVMLICFAVILLQLGVTKVCQHNEVQLGNECCPPCGSGQRVTKVCTDYTSVTCTPCPNGTYVSGLYNCTDCTECNDTEVTIRNCTSTNNTVCASKNYTSFSISGVQHHKQRQNHTAHVTVKQGKSGRHTLARLSLFIFLVGIILLILYLIAAYRSEKCQQCCSIGKIFYRTL

SEQ ID NO: 47 - protein, 186 aa, Artificial Sequence
  Description of Artificial Sequence note = synthetic construct
MDIKNLLTVCTILYISTLVTADIPTSSLPHAPVNGSCDDGEYLDKTHNQCCNRCPPGEFAKIRCSGSDNTKCERCPPHTYTTVPNYSNGCHQCRKCPTGSFDKVKCTGTQNSKCSCLPGWFCATDSSKTEDCRDCIPKRKCPCGYFGGIDELGNPLCKSCCVGEYCDDIRNHRVGPFPPCKLSKCN

SEQ ID NO: 48 - protein, 186 aa, Artificial Sequence
  Description of Artificial Sequence note = synthetic construct
MDIKNLLTACTIFYITTLATADIPTSSLPHAPVNGSCDEGEYLDKRHNQCCNRCPPGEFAKVRCNGNDNTKCERCPPHTYTAIPNYSNGCHQCRKCPTGSFDKVKCTGTQNSKCSCLPGWYCATDSSQTEDCRDCIPKRRCPCGYFGGIDEQGNPICKSCCVGEYCDYLRNYRLDPFPPCKLSKCN

SEQ ID NO: 49 - protein, 148 aa, Artificial Sequence
  Description of Artificial Sequence note = synthetic construct
MMKMTPSYILLVYMFVVVSGDVPYEHINGKCNGTDYNSNSNNLCCKQCDPGMYMTHSCNTTSNTKCDKCPDGTFTSIPNHIPTCLSCRGKCSSNQVETKSCSNTQAEYVSVHPDTTANLKDQMVAGYVYHKQSVILVTAYMATHLKEM

SEQ ID NO: 50 - protein, 167 aa, Artificial Sequence
  Description of Artificial Sequence note = synthetic construct
MTKVIIILGFLIINTNSLSMKCEQGVSYYNSQELKCCKLCKPGTYSDHRCDKYSDTICGHCPSDTFTSIYNRSPWCHSCRGSCGTNRVEVTPCTPTTNRICHCDSNSYCLLKASDGNCVTCAPKTKCGRGYGKKGEDEMGNTICKKCRKGTYSDIVSDSDQCKPMTR

SEQ ID NO: 51 - protein, 289 aa, Artificial Sequence
  Description of Artificial Sequence note = synthetic construct
MMLFILFLLPITVHTATDCPPGYYISKVYPAGTPMCSPCSPGTYTGLQNSLRKCLRCSTCSHNEEPKVACSTTSDVQCQCRQGYYYDPESEMCFPCSNCESSKVKVTTCNRTHDTVCKCKEGYYDKNGVCVKCGNCYLGEGVKSKCANNTDVTCELCKNGTFSDKVSSSNICYLYTMCTLGLTQLNFNVTWFDTICINCSMSTDLLDLETFFTINFVTQRRFPEDDLKNMFRLTYNKTRNDIDSANRDDVEGSFTYDPNLPYTMKQLDYLIASKFLMTAYKKISQICQL

SEQ ID NO: 52 - protein, 320 aa, Artificial Sequence
  Description of Artificial Sequence note = synthetic construct
MMKMTPSYILLVYMFVVVSGDVPYTPINGKCNGTDYNSNNLCCKQCNPGMYMTHSCNTTSNTKCDKCPDDTFTSIPNHSPACLSCRGKCSSNQVETKSCSNTQDRVCVCASGYYCEFEGSNGCRLCVPQTKCGSGYGVYGYSSKGDVICKKCPGNIDKCDLSFNSIDVEINMYPVNKTSCNSSIGSSSTISTSELTITLTHEDCTPVFIGDYYSVVDKLATSGFFTNDKVHQDLTTQCKINLEIKCNSGRESRQLTPTTKVYFMPHSETVTVVGDCLSNLDVYIVYANTDAIYSDMDVVAYHTSYILNVDHIPPNDCERD

SEQ ID NO: 53 - protein, 349 aa, Artificial Sequence
  Description of Artificial Sequence note = synthetic construct
MKSVLYSYILFLSCIIINGRDVTPYAPSNGKCKDNEYKRHNLCCLSCPPGTYASRLCDSKTNTQCTPCGSGTFTSRNNHLPACLSCNGRCDSNQVETRSCNTTHNRICECSPGYYCILKGSSGCKACVSQTKCGIGYGVSGHTSAGDVICSPCGLGTYSRTVSSADKCEPVPSNTFNYIDVEINLYPVNDTSCTRTTTTGISESISTSELTITMNHKDCDPVFREEYFSVLNKVATSGFFTGENRYQNISKVCTLNFEIKCNNKGSSSKQLTKAKNDDGIMPHSETVTLVGDCLSSVDIYILYSNTNTQDYETDTISYHAGNVLDVDSHMPGSCDIHKLITNSKPTHFL

SEQ ID NO: 54 - protein, 348 aa, Artificial Sequence
  Description of Artificial Sequence note = synthetic construct
MRSVLYSYILFLSCIIINGRDLAPHAPSNGKCKDNEYRSRNLCCLSCPPGTYASRLCDSKTNTQCTPCGSDTFTSHNNHLQACLSCNGRCDSNQVETRSCNTTHNRICECSPGYYCLLKGSSGCRTCISKTKCGIGYGVSGYTSTGDVICSPCGPGTYSHTVSSTDKCEPVTSNTFNYIDVEINLYPVNDTSCTRTTTTGLSESISTSELTITMNHKDCDPVFRAEYFSVLNNVATSGFFTGENRYQNTSKICTLNFEIKCNNKDSSSKQLTKTKNDTIMPHSETVTLVGDCLSSVDIYILYSNTNTQDYETDTISYHMGNVLDVNSHMPASCDIHKLITNSQNPTHL

SEQ ID NO: 55 - protein, 349 aa, Artificial Sequence
  Description of Artificial Sequence note = synthetic construct
MKSVLYLYILFLSCIIINGRDAAPYTPPNGKCKDTEYKRHNLCCLSCPPGTYASRLCDSKTNTQCTPCGSGTFTSRNNHLPACLSCNGRCNSNQVETRSCNTTHNRICECSPGYYCLLKGSSGCKACVSQTKCGIGYGVSGHTSVGDVICSPCGFGTYSYTVSSTDKCEPVPNNTFNYIDVEITLYPVNDTSCTRTTTTGLSESILTSELTITMNHTDCNPVFREEYFSVLNKVATSGFFTGENRYQNISKVCTLNFEIKCNNKGSSFKQLTKAKNDDGMMSHSETVTLAGDCLSSVDIYILYSNTNAQDYETDTISYRVGNVLDDDSHMPGSCDIHKLITNSKPTRFL

SEQ ID NO: 56 - protein, 547 aa, Artificial Sequence
  Description of Artificial Sequence note = synthetic construct
MKILLLNENPVVSRLVSLSAKKMSYDFEELNAYSENLGNYDVIVVDSDTPAPLKILKEKCDRLIFLAPRNQNVEDIDAQILQKPFLPTDFLNLLNNKDANKHTSIDLPMLSNDENPYADISLDLDNLNLDDLPDENSLDINSEGMEDLSFDDDAQDDNANKTLETQNLEHDNLEQETIKEQTQEDTQTDLDLTLEDSESEKEDLSQEHTALDTEPSLDELDDKNDEDLEIKEDDKNEEIEKQELLDDSKTNTLEMQEELSESQDDNANKTLETQNLEHDNLEQETIKEQTQEDTQTDLDLTLEDGESEKEDLSQEHTALDTEPSLDELDDKNDEDLEDNKELQANISDFDDLPEVEEQEKEMDFDDLPEDAEFLGQAKYNEESEENLEEFAPVVEEDVQDEIDDFASNLSTQDQIKEELAQLDELDYGIDSDNSSKVLEDFKDEPILDDKELGTNEEEVVVPNLNISDFDTLKESDIQEALGEEILEKNEEPIVSDVTKDDNSEEIVNELSQSIAGAITSSIKDDTLKAALKGMNMNININISFKED

SEQ ID NO: 57 - protein, 496 aa, Artificial Sequence
  Description of Artificial Sequence note = synthetic construct
MLRCRVADVVSSDQMCEMTNFPPVQATVGYVQLLRRLSFKREFFVPFVPTKHVIFTFQTRNPLEEGKVKDEVTDLVSHYFNWIRERGVYSFQILQLYVCILSGCTVVVIECPDTLTTLDVHSFSQEPEAPTVGKRRVLVFPCDNDEISDSELESNYYIVPTCTLCAERLEPTLTGYSSPTCSCVDGRECRCLLEQSSCVVCQTSITMQHESQKVQCEQCSRTGDPWICLVCGYVGCSRYQAKHAREHYLQHKHLFSMSLLTQQIWDYDSDAFVHRVVVLLDNATGAVNRVQYPDRDNIPSSLADEYVDAAAEKVSKKHINAKFDSKVETSNEQLALMIISELNTRRVEYETEMHGGSHHLNDELMGDPSLCSVIVAERACAASRERWWQLHNANKSIQEELMQRRREEEAHQKTIDELQQELRSVVQHYATREYSLLTEINELRQTITDVETNLRTFAKLSRGLGNDTLEHVRIVGGTKEPKPRRRGGNNTRDGRA

SEQ ID NO: 58 - protein, 680 aa, Artificial Sequence
  Description of Artificial Sequence note = synthetic construct
MEIFETILIFIAVVILSSFVHTFIPKVPLAFIQIFLGMLLFITPIPVQFNFDSELFMVTMIAPLLFVEGVNVSRVHLRKYIKPVMMMALGLVITTVIGVGLFIHWIWPDLPIGAAFAIAAILCPTDAVAVQAITKGKVLPKGAMTILEGESLLNDAAGIISFKIAVGVLVTGAFSLVDAVQLFLIASIGGAVVGLLIGMALVRFRLTLMRRGYENINMFTIIQLLTPFVTYLIAELFHASGIIAAVVAGLVHGFERDRIMQVRTQLQMSYNHTWNILGYVLNGFVFSILGFLVPEVIIKIIKTEPHNLIFLIGITIVVALAVYLFRFVWVYVLYPYFYLAISPFQKMMTKNDDDNPTTEKPPKRSLYALIMTLCGVHGTISLAIALTLPYFLAGHHAFTYRNDLLFIASGMVIISLVVAQVLLPLLTKPAPKTVIGNMSFKVARIYILEQVIDYLNQKSTFETSFKYGNVIKEYHDKLAFLKTVEKDDENSKELERLQKIAFNVETKTLESLVDEGQITNSVLENYMRYAERTQVYRQASLIRRMIVLLRGALLKRRVQTRVNSASSLSVTDNLMELNKINKLVHYNVVSRLSKETTKDNTLEIGMVCDGYLMRIENLTPSNFFNSASEDTITKIKLNALREQRRILRELIDTDEVSEGTALKLREAINYDEMVIVDSMT

SEQ ID NO: 59 - protein, 294 aa, Artificial Sequence
  Description of Artificial Sequence note = synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEFLVPHLGDREKRDSVCPQGKYIHPQNNSICCTKCHKGTYLYNDCPGPGQDTDCREFPGRLERPHRD

SEQ ID NO: 60 - protein, 286 aa, Artificial Sequence
  Description of Artificial Sequence note = synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEFYAPEPGSTCRLREYYDQTAQMCCSKCSPGQHAKVFCTKTSDTVCDEFPGRLERPHRD

SEQ ID NO: 61 - DNA, 20 bp, Artificial Sequence
  Description of Artificial Sequence note = synthetic construct
tccatgacgttcctgatgct

(19) United States
(12) Patent Application Publication
(10) Pub. No.: US 2010/0041596 A1
(43) Pub. Date: Feb. 18, 2010

(54) Amelioration of Inflammatory Arthritis By Targeting the Pre-ligand Assembly Domain (Plad) of Tumor Necrosis Factor Receptors

(76) Inventors: Michael Lenardo, Potomac, MD (US); Guo-Min Deng, Bethesda, MD (US); Francis Ka-Ming Chan, Silver Spring, MD (US); Lixen Zheng, Rockville, MD (US)

Correspondence Address: NATIONAL INSTITUTE OF HEALTH;C/O Ballard Spahr LLP, SUITE 1000, 999 PEACHTREE STREET, ATLANTA, GA, 30309, US

(21) Appl. No.: 11/922,547

(22) PCT Filed: Jun. 26, 2006
(86) PCT No.: PCT/US06/24909

§ 371 Date: Mar. 27, 2008

Related U.S. Application Data
(60) Provisional application No. 60/694,015, filed on Jun. 24, 2005.
(60) Provisional application No. 60/717,589, filed on Sep. 16, 2005.

(51) Int. Cl. A61K 38/16; C07K 14/00; C07H 21/04; C12N 15/63; C12N 5/02; C12Q 1/02

(52) U.S. Cl. 514/12; 530/324; 530/350; 536/23.1; 435/320.1; 435/375; 435/29

(57) ABSTRACT

The present invention provides a polypeptide comprising the isolated amino acid sequence of a pre-ligand assembly domain (PLAD) of a TNF receptor-like receptor. Also provided by this invention is a polypeptide comprising the isolated amino acid sequence of a pre-ligand assembly domain (PLAD), wherein the PLAD is selected from the group consisting of: the PLAD of a TNF-R, the PLAD of p60, the PLAD of p80, the PLAD of Fas (CD95/APO-1), the PLAD of TRAIL receptors, the PLAD of LTyR, the PLAD of CD40, the PLAD of CD30, the PLAD of CD27, the PLAD of HVEM, the PLAD of OX40 and the PLAD of DR4. TNF-R, p60, p80, Fas, TRAIL receptor, LTyR, CD40, CD30, CD27, HVEM, OX40, DR4, TROY, EDAR, XEDAR, DCR3, AITR, 4-1BB, DR3, RANK, TACI, BCMA, DR6, DPG, DR5, DCR1 AND DCR2 are all members of the TNF receptor superfamily or the TNF-like receptor family. The invention also provides the PLAD for other members of the TNF receptor superfamily. The polypeptides of the present invention can be utilized to inhibit oligomerization of members of the TNF receptor superfamily. These polypeptides can also be utilized to inhibit ligand binding to members of the TNF receptor superfamily. The present invention also provides a composition comprising an inhibitor of TNF receptor oligomerization. Further provided by this invention are members of the TNF receptor superfamily that are lacking a PLAD.

This application claims priority to U.S. Provisional Application No. 60/694,015, filed on Jun. 24, 2005, and U.S. Provisional Application No. 60/717,589, filed on Sep. 16, 2005, hereby incorporated by reference in their entirety.

### BACKGROUND OF THE INVENTION

1. Field of the Invention

This invention provides a novel function for a conserved domain in the extracellular region of the members of the TNF receptor (TNFR) superfamily in mediating specific ligand-independent assembly of receptor oligomers.

2. Background Art

The members of the TNFR superfamily typically contain one to six cysteine rich domains (CRDs) in their extracellular regions, a single transmembrane domain and variably sized intracytoplasmic domains. The members of this receptor family typically bind to ligands of the TNF cytokine family that are defined by structural, functional and sequence similarities. These receptors form trimers in their active liganded state and several members contain a cytoplasmic domain referred to as a death domain.

According to the present invention, the extracellular region of these receptors is further characterized by a novel self-association or homotypic association function that is mediated via a pre-ligand receptor assembly domain (PLAD) that contains at least one cysteine rich domain. More specifically, members of the TNFR superfamily, including TRAIL receptor 1, CD40, 60 kDa TNFR and 80 kDa TNFR show this homotypic association. Other members of the TNFR superfamily, including Fas, LTβR, CD40, CD30, CD27, HVEM, RANK, OX40 and DR4 contain this PLAD. The PLAD is necessary for ligand binding and receptor function. Thus, members of the TNFR superfamily appear to signal through distinct pre-formed complexes rather than through ligand-induced cross-linking of individual receptor subunits. Therefore, PLAD can be targeted by pharmaceutical agents in order to block the formation of these preformed complexes and thus block receptor function.

It is well established that many microbes have evolved effective strategies for countering the immune response directed against them (79). More specifically, several viruses and bacteria express homologs of cellular proteins designed to modulate or directly block the action of immune effector molecules, including cytokines and chemokines (80). Viral homologs of TNF receptor-like receptors (vTNFRs) have been identified for several large DNA viruses, including several poxviruses and human cytomegalovirus. The existence of or any role for PLAD-mediated self-association of vTNFRs or heterologous association with TNF family receptors has not been elucidated.

Two major arthritides are rheumatoid arthritis (RA) and septic arthritis (SA). RA is a common human autoimmune disease with chronic joint inflammation and progressive bone destruction (48). Although the etiology and pathogenesis of RA are not yet fully understood, cytokines such as TNF-α, IL-1, IL-6 and receptor activator of NF-κB ligand (RANKL), are involved in disease progression (56-60). Nuclear factor-kappa B (NF-κB) is a critical regulator of these cytokines (56-60). TNF-α plays a key role in the pathogenesis of RA and its antagonists such as etanercept (also known as Enbrel), a TNFR II immunoglobulin Fc fusion protein, and infliximab (also known as Remicade), an anti-TNF-α monoclonal antibody, can improve the clinical course of RA (48). SA is a rapidly progressive and highly destructive joint disease induced by bacterial infection in which TNF-α also plays an important role (49). Experimental mouse models of arthritis induced by TNF-α (59), lipopolysaccharide (LPS), CpG-DNA (50, 61), and collagen (62) have been useful for testing new treatments. These agents induce synovitis, pannus formation, and bone and cartilage destruction as well as other features observed in human RA and SA.

According to the invention, in vitro and in vivo data indicate that TNFR PLAD proteins can potently inhibit TNF-α and its consequences in experimental inflammatory arthritis.

### SUMMARY OF THE INVENTION

The present invention provides a polypeptide comprising the isolated amino acid sequence of a pre-ligand assembly domain (PLAD) of a TNF receptor-like receptor.

Also provided by this invention is a polypeptide comprising the isolated amino acid sequence of a pre-ligand assembly domain (PLAD), wherein the PLAD is selected from the group consisting of: the PLAD of TNF-R, the PLAD of p60, the PLAD of p80, the PLAD of Fas (CD95/APO-1), the PLAD of TRAIL receptors, the PLAD of LTβR, the PLAD of CD40, the PLAD of CD30, the PLAD of CD27, the PLAD of HVEM, the PLAD of OX40, the PLAD of DR4, the PLAD of NGFR, the PLAD of Troy, the PLAD of EDAR, the PLAD of XEDAR, the PLAD of DcR3, the PLAD of AITR, the PLAD of 4-1BB, the PLAD of DR3, the PLAD of RANK, the PLAD of TACI, the PLAD of BCMA, the PLAD of DR6, the PLAD of OPG, the PLAD of DRS, the PLAD of DcR1, and the PLAD of DcR2. TNF-R, p60 TNFR, p80 TNFR, Fas, TRAIL receptors, LTβR, CD40, CD30, CD27, HVEM, OX40, DR4, NGFR, Troy, EDAR, XEDAR, DcR3, AITR, 4-1BB, DR3, RANK, TACI, BCMA, DR6, OPG, DRS, DcR1, and DcR2 are all members of the TNF receptor superfamily also referred to herein as the TNF receptor-like receptor family. The invention also provides the PLAD for other members of the TNF receptor superfamily and how it can be identified by one of skill in the art.

The polypeptides of the present invention can be utilized to inhibit PLAD self-association as well as oligomerization of members of the TNF receptor superfamily. These polypeptides can also be utilized to inhibit ligand binding to members of the TNF receptor superfamily.

The present invention also provides a composition comprising an inhibitor of TNF receptor oligomerization. Further provided by this invention are members of the TNF receptor superfamily that are lacking a PLAD.

### BRIEF DESCRIPTION OF THE DRAWINGS

FIG. 1A illustrates TNFR oligomers in the absence of ligand. H9 T cell lymphoma, treated or untreated with TNFα, were subjected to crosslinking with DTSSP (7). Total cell lysates were electrophoresed under non-reducing (lanes 1-4, 9-12) or reducing (lanes 5-8, 13-16) conditions as indicated and blotted for p60 or p80 TNFRs. The brackets indicate the position of trimers (T) and monomers (M). The circles indicate a non-specific protein cross-reacting with the anti-p80 antibody. The results represent three independent experiments.

FIG. 1B illustrates specific p60 TNFR self-association. 293T cells were transfected with p60ΔCD-GFP-HA (lanes 1-3) or pEGFP-N1 (lanes 4-6) and either pcDNA3 (lanes 1, 4), p60ΔCD-HA (lanes 2, 5) or HVEMΔCD-HA (lanes 3, 6). Immunoprecipitation was carried out with anti-GFP antibody (GFP IP in the top 2 panels) and blotted with anti-HA antibody (HA WB) or anti-GFP antibody (GFP WB) as indicated. The top and middle panels show the precipitated p60ΔCD-GFP-HA (or GFP) and p60ΔCD-HA respectively. The bottom panels show the p60ΔCD-HA and HVEMΔCD-HA proteins in cell lysates. Results represent five experiments.

FIG. 1C illustrates specific p80 self-association and the definition of the Pre-Ligand Assembly Domain (PLAD). 293T cells were transfected with the plasmids indicated at the top. Immunoprecipitation was performed with a C-terminal-specific anti-p80 antibody (p80 IP) that recognizes only the full-length p80 (top and middle panels). The expression of the truncated p80 or p60 proteins in the lysates is shown in the bottom panel. Western blots were performed with anti-HA antibody (top and bottom panels) and the C-terminal specific anti-p80 antibody (middle panel). The open circles represent the glycosylated and unglycosylated forms of p80. The closed circle denotes the Ig heavy chain.

FIG. 1D illustrates that PLAD is sufficient for receptor self-association. 293T cells were transfected with p80ΔCD-GFP-HA (lanes 1-5) together with the plasmids indicated at the top of each lane. Immunoprecipitation was performed with anti-GFP antibody and Western blots with anti-HA antibody. The co-precipitated DCD proteins and their expression in total cell lysates are shown in the middle and bottom panels respectively. The top panel shows the precipitated p60ΔCD-GFP-HA protein.

FIG. 1E illustrates the PLAD is essential for TNFα binding. Histograms show the expression of transfected receptors (by anti-HA staining) and their binding to TNFα in 293T cells transfected with the indicated constructs (25). The x-axis shows the intensity of fluorescence and the y-axis shows the cell number. The numbers shown are percentages of positive population compared to the vector-transfected control.

FIG. 2A illustrates that replacement of residues in the PLAD prevents self-association. 293T cells were transfected with the indicated plasmids. Immunoprecipitation was performed as in FIG. 1 with anti-GFP antibody. Western blots were performed with anti-HA antibody. The top and middle panels show the precipitated p60ΔCD-GFP-HA (open circle) and p60ΔCD-HA mutant proteins (bracket) respectively. The bottom panel shows the expression of p60ΔCD-HA mutants (bracket) and HVEMΔCD-HA (filled circle) in cell lysates.

FIG. 2B illustrates homotypic self-association of p60 and p80 TNFRs as demonstrated by fluorescence resonance energy transfer (FRET). Histograms of flow cytometric analysis of 293T cells transfected with the indicated CFP (top) and YFP (bottom) plasmid pairs. The dashed line represents the CFP transfected alone control, the solid line represents FRET without TNFα and the thick line represents FRET with TNFα. The x-axis and y-axis show the FRET fluorescence intensity and cell number respectively. FRET was analyzed in the CFP positive population in which all cells were YFP positive as well. FRET is defined as fluorescence emission of YFP due to excitation of CFP. The results are representative of four independent experiments.

FIG. 3A shows a sequence alignment of CRD1 for representatives (CRD1 of p60 (SEQ ID NO: 22), CRD1 of p80 (SEQ ID NO: 23), CRD1 of LTβR (SEQ ID NO: 24), CRD1 of CD40 (SEQ ID NO: 25), CRD1 of HVEM (SEQ ID NO: 26) and CRD1 of CD30 (SEQ ID NO: 27) of the TNFR superfamily (26). This figure illustrates the highly conserved positions of cysteines that for disulfide bonds and define the cysteine-rich domain which confers membership in the TNFR superfamily.

FIG. 3B illustrates receptor self-association in other TNFR superfamily members. 293T cells were transfected with either DR4ΔCD-GFP-HA (lanes 1-4) or CD40ΔCD-GFP-HA (lanes 5, 6) together with p80ΔCD-HA (lanes 1,6), p60ΔCD-HA (lane 2), HVEMΔCD-HA (lane 3), DR4ΔCD-HA (lane 4) or CD40ΔCD-HA (lane 5). Immunoprecipitations and Western blots were performed with anti-GFP and anti-HA antibodies respectively. The top panels show the precipitated proteins in the immune complexes and the bottom panels show the expression of the DCD proteins in the cell lysates. The filled circles denote the GFP fusion proteins and the arrows indicate the DCD protein in the immune complexes.

FIG. 3C shows flow cytometric analysis of specific receptor association of DR4 and CD40 as demonstrated by FRET. Transfections with the indicated CFP (top) and YFP (bottom) plasmid pairs were performed as in FIG. 2B. The dashed lines represent background FRET with CFP alone and the thick lines represent FRET in the presence of both CFP and YFP fusion proteins. For each group, the x-axis is the FRET intensity and the y-axis is the cell number.

FIG. 3D illustrates the two models of TNFR signaling based on pre-associated trimer complexes. For the pre-assembly chain rearrangement model (left), the ovals represent CRDs (CRDs are numbered 1-4 going from membrane distal to membrane proximal) and stippled boxes indicate the cytoplasmic domains. The receptors are viewed perpendicular to the plasma membrane. The Roman numerals represent the chains in the trimer complex. For the trimer clustering model (right), the gray symbols indicate pre-assembled TNFR trimers on the cell surface and the encircled triangles represent the trimeric TNFα. The numbers 1-3 represent the three chains of receptor in the pre-assembled trimer complex. The receptors are viewed top down to the plasma membrane.

FIG. 4A snows that a pathogenic Fas mutation causes dominant-interference in the absence of ligand binding. Surface expression and binding characteristics of wild-type (WT), Pt 2 (del 52-96), and Pt 6 (A241D) Fas molecules. The left column shows surface expression 24 hours after transfection into 293T cells using staining for the AU-1 epitope tag present at the N-terminus of each receptor protein. The middle column shows the same cells stained with 10 μg/ml of the anti-Fas agonistic antibody APO-1 (Kamiya). The right column shows binding of FasL engineered to trimerize through a modified leucine zipper and visualized by staining with an anti-leucine zipper mAb (FasL stain). Antibody binding was visualized with phycoerythrin-conjugated anti-mouse antibodies. The brackets indicate the percentage of cells strongly positive for staining when compared to the non-transfected controls. In each plot the thick and thin lines represent the signals from the transfected and non-transfected cell preparations, respectively. All histograms represent 10,000 events plotted on a 4 decade logarithmic fluorescence scale (X axis) vs. cell count (Y axis). Data was collected on a FACScalibur flow cytometer using Flowjo software (Treestar software).

FIG. 4B shows dominant inhibition by mutant Fas molecules co-transfected with WT Fas. Ten μg of the indicated expression vectors or pCI vector alone were electroporated into BW cells lacking human Fas as previously described (15), with 5 μg of pEGFP-N1 (Clontech) to mark transfected cells with GFP. Twenty-four hours later, the indicated amounts of anti-Fas mAb APO-1 were added along with 1/20 volume soluble protein A (Sigma) for maximal apoptosis induction. Apoptosis was quantitated by enumerating GFP-positive viable cells by flow cytometry and calculating cell loss (15).

FIG. 4C illustrates self-association of Fas molecules. An expression vector encoding HA-tagged Fas with the C-terminal death domain replaced by the Green Fluorescent Protein (HAFas 1-210:GFP) was co-transfected with wild-type (WT) Fas and the EC mutant Pt 2 Fas (del 52-96). Control cells were co-transfected with WT Fas and an HA-tagged cytoplasmic truncated version of the TNF-receptor family member Herpesvirus Entry Mediator (HVEM or HveA) fused to GFP (HA HVEMΔCD:GFP). Cell lysates were lysed, immunoprecipitated with anti-GFP and electrophoresed as described in the Examples. Blots of precipitated proteins were probed with a polyclonal antiserum against the Fas C-terminal (C20, Santa Cruz biotechnology) (Anti-Fas CT) to reveal full-length Fas molecules co-precipitating with the GFP tagged proteins. Cell lysates were also probed in western blots (WB) with anti-HA-HRP (Roche Molecular Biochemicals) and anti-Fas C20 to quantitate the total amount of these proteins. The open circle indicates the IgG heavy chain in the immunoprecipitates, the closed circle indicates WT Fas, and the arrow indicates the truncated Pt 2 Fas protein. The upper band in some lanes blotted with anti-Fas C20 represents glycosylated Fas.

FIG. 5A shows the expression and function of Fas mutants lacking the PLAD or ligand binding. Binding of APO-1 and FasL by N-terminal Fas mutants. Staining of the indicated HA-tagged Fas mutants, the R86S Fas mutant, and control transfections with a C-terminal truncated HA-tagged TNFR2 was performed as in FIG. 1A except that anti-HA was used instead of anti-AU1 to show total expression of each mutant on the cell surface.

FIG. 5B shows the interaction of Fas extracellular domains is dependent on a domain in the N-terminal region of the protein. In lanes 14, 293T cells were co-transfected with an AU-1 tagged Fas 1-210 lacking the death domain and the indicated HA-tagged Fas mutants or control TNFR2 protein (HA TNFR2ΔCD). Lysates were immunoprecipitated with anti-AU1, and probed with anti-HA to reveal co-precipitated proteins. Control blots with an antibody against the N-terminal of Fas (WB anti-FasN) are shown to quantitate the amount of the AU-1 Fas 1-210 protein in the lysates. The results are representative of three independent transfections. Lanes 5-7 show co-precipitation of WT Pas and the FasR86S mutant by HAFas1-210:GFP with the same procedure used in FIG. 1C. The open circle indicates the Ig heavy chain of the immunoprecipitating antibody, and the closed circle indicates the position of immunoprecipitated Fas.

FIG. 5C illustrates the induction of apoptosis is lost in Fas molecules lacking the self-association domain. BW5147 murine thymoma cells were transfected with 10 μg of expression vectors for indicated Fas molecules. Apoptosis was induced with 500 μg/ml soluble APO-1 and quantitated as in FIG. 1B.

FIG. 5D illustrates the induction and inhibition of apoptosis by the non-ligand binding R86S Fas mutant. BW cells were transfected with 10 μg of each Fas expression vector and 5 μg of GFP plasmid. Apoptosis induction and quantitation was performed as in FIG. 2C, except that APO-1 was used to induce apoptosis in samples shown with open bars, and 5% v/vol FasL supernatant was added to the samples with filled bars.

FIG. 6A shows Fluorescence Resonance Energy Transfer between Pas molecules. Dot plots showing the relationships between CFP, YFP and FRET signals in the indicated co-transfectants. CFP and YFP fusion proteins were constructed, transfected into 293T cells and analyzed on a FACS vantage cytometer. Numbers are the percentage of cells positive for CFP or YFP with FRET signal (top right quadrant).

FIG. 6B is a comparison of FRET signals between full-length and N-terminal deleted Fas receptors. Histograms of FRET signals were generated in cells gated for CFP fluorescence. YFP fluorescence was comparable between all transfectants. The thick line is the signal from co-transfected cells and the thin line is the signal from the CFP construct alone of each pair.

FIG. 6C shows FRET efficiency for the indicated CFP and YFP pairs as determined by microscopic photobleaching of YFP on individual cells (Five readings of 4-7 cell regions). The numbers represent the average E % and standard error for each plasmid pair.

FIG. 7A illustrates pre-association of endogenous Fas receptor chains. 1×10⁷ H9 lymphoma cells were treated with the crosslinker DTSSP (Pierce, 10 mM for 30 minutes at 4° C., followed by quenching with 10 mM Tris-Cl pH8 for 15 min), and/or stimulated with 1 μg of the agonistic antibody APO-1 or FasL for 15 minutes under the indicated conditions. For anti-Fas immunoblotting, cell lysates were treated with N-glycanase-F (Roche Molecular Biochemicals) before electrophoresis and probed with the anti-Fas C terminal mAb B10 (Santa Cruz Biotechnology) and anti-mouse IgG1-HRP (Southern Biotechnology).

FIG. 7B After treatment with the indicated reagents, cells were lysed, immunoprecipitated and blotted for FADD and caspase-8 as previously described (11). The positions of the two isoforms of procaspase-8 (p54/52) and the caspase-8 cleavage products after proteolysis of the p11 caspase subunit (p43/41) are shown with arrows.

FIG. 7C shows PARP cleavage. Aliquots of cells used in (A) and (B) were cultured at 37° C. for an additional 4 hrs and cell lysates were blotted with anti-PARP mAb (Research Diagnostics Inc). The upper band is the 115 kD full-length PARP and the lower band is the signature 85 kD caspase cleavage fragment. The results are representative of at least three independent experiments for each condition.

FIG. 8A illustrates that dominant interference depends on the N-terminal PLAD. Alignment of selected ALPS patient Fas mutations from families studied at the NIH. “X” symbols indicate the location of point mutations. Capacity to associate with wildtype Fas as tested by co-precipitation (SA) and dominant inhibition of Fas-induced apoptosis in co-transfection studies (DI) are indicated as shown. Sequences encoding dominant-negative PLAD containing polypeptides encoded by mutations from patients #1 and #20 are shown. Numbering begins with the first amino acid after the signal peptide. Italics denote extra amino acids added by frameshift mutations.

FIG. 8B shows that dominant interference is lost without the PLAD. Fas-sensitive Jurkat T lymphoma cells were transfected with the 10 μg of the indicated constructs and 2.5 μg of the GFP reporter plasmid. Eighteen hours after transfection, the indicated amounts of Apo-1 were added for 6 hours and apoptosis was quantitated by staining with Annexin V-PE (Pharmingen). Percentages are the percent of GFP(+) cells staining positive for Annexin V. These results are representative of three independent transfections.

FIG. 9 shows the analysis of immunoprecipitates for the presence of p80 chimeric receptors or truncations. 293T cells were transfected with the indicated plasmids and harvested for co-immunoprecipitation using an anti-p80 COOH-terminal specific antibody. The immunoprecipitates were analyzed for the presence of p80 chimeric receptors or truncations using anti-HA antibody in Western blot analysis (top panel). The bottom panel shows the expression of the HA-tagged proteins in whole cell lysates.

FIG. 10 shows expression of recombinant bacterial PLAD proteins. (a) A model of how the PLAD contributes to receptor trimer assembly and competence for ligand binding. A soluble PLAD protein could associate with individual receptor chains, prevent trimeric receptor assembly, and thereby block ligand-induced signaling. (b) Gel electrophoresis of purified GST, P60 PLAD-GST (P60), and P80 PLAD-GST (P80). Molecular weight markers and sizes in kilodaltons are shown on the left. (c) Western blot analysis using monoclonal antibodies (MAb) against the P60 PLAD (top panels) or the P80 PAD (bottom panels). Titrations in μg protein used in the original gel electrophoresis for P60 PLAD (left panels) or P80 PLAD (right panels) are indicated.

FIG. 11 shows the effects of PLAD proteins in TNF-α-induced cell death. (a) Cell death assessed by flow cytometry after L929 cells were treated with: medium; human TNF-α (hTNF) (2 ng); hTNF-α (2 ng)+P60 PLAD (P60) (40 μg). Inset shows phase contrast photomicrographs. In the lower right is given the percent of gated live cells. The Y axis is propidium iodide (PI) staining and the X axis is FSC (forward scatter profile). Dead cells exhibit increased PI staining and reduced FSC. (b) Cell loss induced by mouse TNF-α (mTNF) (2 ng) or hTNF (2 ng) with different doses of PLAD P60 (P60) protein. (c) Loss of L929 cells induced by mTNF (2 ng) or hTNF (2 ng) with different doses of PLAD CD40 (CD40) protein. (d) Loss of 42.3 Jurkat cells after treatment with TNF-α or anti-Fas with and without P60 PLAD (P60), P80 PLAD (P80) and GST for 12 h. TNF-α (3 ng), P60L (low; 4.5 μg), P60H (high; 15 μg), P80L (low; 4.5 μg), P80H (high; 15 μg), GST (15 μg), anti-Fas (10 ng). (e) Caspase-8 activity in L929 cells measured by optical density (OD) of substrate conversion treated with mTNF-α (4 ng) with or without P60 PLAD (100 μg), etanercept (25 μg), infliximab (20 μg).

FIG. 12 shows the effects of P60 and P80 PLAD proteins on arthritis induced by intra-articular injection of TNF-α in BALB/c mice and bacterial CpG DNA in C3H/HeJ mice. (a) Representative photomicrographs of H&E-stained tissue sections of knee joints showing: PBS; 45 ng TNF-α; P60 PLAD (100 μg) and 45 ng TNF-α; P80 PLAD (100 μg) and 45 ng TNF-α; CpG DNA (1 nmole); CpG DNA (1 nmole) and P60 PLAD (100 μg). Arrows indicate foci of inflammation. Labels are: C (cartilage), JC (joint cavity), ST (synovial tissue), B (bone), and the arrowhead indicates the lining layer of synovial tissue. (b, c) Quantitation of the histological analysis of synovitis, pannus, and erosion of bone and cartilage of experimental groups (n=5) treated with TNF-α alone (TNF) or TNF-α plus the P60 PLAD protein (P60) or TNF-α plus P80 PLAD protein (P80) as indicated. (d) Quantitation of histological analysis of synovitis, pannus, erosion of bone and cartilage of each experimental group (n=5). These analyses were repeated at least two times. CpG DNA alone (CpG), CpG DNA plus P60 PLAD (P60). Values are mean±standard deviation (s.d.) **P<0.01 for treated versus control group. Mice were sacrificed 3 d after intra-articular inoculation for histopathological examination. This is representative of three experiments.

FIG. 13 shows the effects of P60 and P80 PLAD proteins on CIA in DBA/1J mice. A masked experiment (a-f). (a) Photographs of the paws of CIA mice treated with PBS or P60 PLAD. (b) H&E stained section of a CIA joint 75 d after primary immunization treated with PBS or 4 weeks of intraperitoneal injection of P60 PLAD protein (100 μg three times per week). (c) Severity of arthritis; paw thickness measurement; and weight in CIA mice treated with PBS (n=13 mice) (square), P60 PLAD protein (P60) (n=12 mice) (diamond), and P80 PLAD protein (P80) (n=13 mice) (oval). (d) Incidence of arthritis in PBS, P60, and P80 PLAD treatment groups. (e) Evaluation of synovitis; pannus; erosion of bone and cartilage in joint sections in CIA treated for 4 weeks as indicated. (f) IL-1 and IL-6 level in sera. *=P<0.05 versus control PBS group. (g) Severity of arthritis in CIA mice receiving 2 weeks' intraperitoneal injection of PBS (n=10 mice) (square), P60 PLAD protein (P60) (n=10 mice) (diamond), and etanercept (n=10 mice) (oval). *=P<0.05 versus control PBS group. (h) The effect of P60 PLAD protein in established CIA. Severity of arthritis in established CIA mice receiving 2 weeks' intraperitoneal injection of PBS (n=9 mice) (square), P60 PLAD protein (P60, 400 μg every other day) (n=10 mice) (diamond). *=P<0.05 versus control PBS group.

FIG. 14 shows TNFR expression in arthritic joints and PLAD protein inhibition of TNF-α binding and NF-κB activation. (a) Immunohistochemistry for TNFR1 and TNFR2 in an arthritic joint from DBA/1J mice sacrificed 75 d with CIA treated with PBS or P60 PLAD. Brown color indicates TNFR expressing cells. (b) Flow cytometry following staining with: 50 ng biotinylated human TNF-α (Bt-TNF-α) and different doses of P60 PLAD protein pretreatment. Curves represent Bt-TNFα alone, Bt-TNF-α plus 3 μg P60 PLAD, Bt-TNF-α plus 15 μg P60 PLAD, Bt-TNF-α plus 30 μg P60 PLAD, human TNF-α, or negative control. (c) Gel electrophoresis of 50 ng human TNF-α immunoprecipitated (IP) with etanercept (1 μg), P60 (1 μg) or P80 (1 μg) PLAD protein as indicated (test protein). Western blot using antibody (Ab) against TNF-α. (d) Electrophoretic mobility shift assay of nuclear extracts prepared from A3 Jurkat cells treated with TNF-α in the presence (+) or absence (−) of P60 PLAD protein using radiolabelled oligonucleotide probes for NF-κB (left arrow) or OCT1 (right arrow). Quantitation of NF-κB p65 nuclear translocation in mononuclear cells isolated from spleen from TNFR1(e) or TNFR2 (f) knockout (−/−) mice after these cells were treated with mTNF-α (2 ng) with or without P60 (100 μg) or P80 (100 μg) PLAD protein in vitro. *P<0.05 versus control group; **=P<0.01 versus control group. The difference between the P60 and P80 treatment groups is significantly different in each genetic background (P<0.01).

FIG. 15 shows that P60 PLAD protein inhibits osteoclastogenesis and RANK and RANK ligand (RANKL) expression. (a) Representative photomicrographs of immunohistochemistry of calcitonin receptor in an arthritic joint from DBA/1J mice sacrificed 75 d after primary immunization with collagen treated with PBS or P60 PLAD protein Light shaded arrow indicates positive staining (brown in original color slide). (b) Effects of PLAD protein in TNF-α-induced osteoclastogenesis in vitro. Photomicrographs of TRAP-positive osteoclasts in bone marrow macrophages (MM) cultured with: M-CSF and mouse TNF-α (10 μg); mouse TNF-α (10 μg) plus 32 μg P60 PLAD; or PBS. Light shaded arrows indicate tartrate-resistant acid phosphatase (TRAP)-positive osteoclasts. Quantitation of TRAP-positive cells in each well of BMM cultures after 7 d treatment with different doses of P60 PLAD protein as indicated, determined microscopically. (c) Photomicrographs of immunohistochemistry of RANK and RANKL staining in an arthritic joint from DBA/1J mice sacrificed 75 d after primary immunization with collagen and then treated with PBS, with P60 PLAD protein. Dark (brown in its original color slide) staining indicates positive staining cells.

FIG. 16 shows the amino acid sequences of human PLAD-GST fusion proteins in standard single letter code. (a) Shown bold and underline (amino acid residues 230-282) is the P60 PLAD peptide sequence (SEQ ID NO:59). (b) Shown bold and underline (amino acid residues 230-274) is the P80 PLAD peptide sequence (SEQ ID NO:60). Shown in plain text in both parts is the GST protein sequence.

FIG. 17 shows the effects of P60 PLAD protein on TNF-α-induced cell death in L929 cells. Phase contrast photomicrographs of cells treated for 19 hours with: (a) medium alone; (b) human TNF-α (2 ng); (c) P60 PLAD (3 μg)+hTNF-α (2 ng); (d) P60 PLAD (30 μg)+hTNF-α (2 ng); (e) infliximab (1 μg)+hTNF-α (2 ng); (f) etanercept (1 μg)+hTNF-α (2 ng). (g) P80 PLAD (30 μg)+hTNF-α (2 ng). Magnification 400×. (h) Loss of L929 cells induced by hTNF (2 ng) with or without P60 PLAD (50 μg), etanercept (1 μg) and infliximab (1 μg).

FIG. 18 shows the effects of GST protein and P80 PLAD protein on inflammatory arthritis. (a) Quantitation of the histological analysis of synovitis, pannus and erosion of bone and cartilage in experimental groups (n=5) treated with TNF-α (45 ng) alone (TNF) or TNF-α (45 ng) plus the GST protein (GST, 100 μg) in BALB/c mice; (b) with CpG DNA (1 mmole) alone (CpG) or CpG DNA (1 mmole) plus P80 PLAD protein (P80, 100 μg) in C3H/HeJ mice. Values are mean±s.d. Mice were sacrificed 3 d after intra-articular inoculation for histopathological examination. This is representative of three experiments. (c) Photomicrographs of H&E stained section showing inflammation and destruction of a CIA joint 75 d after primary immunization with 4 weeks' intraperitoneal injection of P80 PLAD protein (100 μg). Magnification 200×. (d) Quantitation of the histological analysis of synovitis, pannus, and erosion of bone and cartilage in CIA in DBA/1J mice treated with P80 PLAD protein for 4 weeks. P>0.05 compared with control group.

FIG. 19 shows photomicrographs of immunohistochemistry. (a) TNFR1 and TNFR2 in an arthritic joint from a TNF-α transgenic mouse sacrificed at 6 months. Arrow in TNFR1 panel shows dark (brown in its original color slide) staining indicating receptor expression. Arrow in TNFR2 panel indicates high TNFR2 expression by chondrocytes deep within cartilage. (b) calcitonin receptor in: an arthritic joint from a TNF-o transgenic mouse sacrificed at 6 months of age, and a healthy joint from a control C57BL/6 mouse. (c) RANK and RANKL staining in an arthritic joint from TNF-α transgenic mice sacrificed at age of 6 months, and in a healthy joint from normal control C57BL/6 mouse. Dark (brown in it's original color slide) staining indicates a positive histochemical reaction. Magnification 300×.

FIG. 20 shows the immunogenicity and half-life of the P60 PLAD protein. (a) Anti-PLAD P60 antibody level compared to a standard curve based on purified PLAD protein in sera from DBA/1 mice sacrificed 75 d after primary immunization with collagen and then treated with PBS, P60 or P80 PLAD proteins. GST+means that GST was added to remove GST antibody from sera. (b) Half-life of P60 PLAD protein. (c) Half-life of P80 PLAD protein.

FIG. 21 shows that PLAD proteins inhibit TNF-α-induced IkBα degradation. Western Blot shows that IkBα degradation in mononuclear cells isolated from spleen from TNFR1 or TNFR2 knockout (−\−) mice after these cells were treated with mTNF-α (2 ng) with or without P60 (10, 50, 100 μg) or P80 (10, 50, 100 μg) for 5 min (b), 15 min (a) or GST (10, 50, 100 μg) for 1 h (c) in vitro.

FIG. 22 shows gel electrophoresis of 50 ng of human TNF-α immunoprecipitated (IP) with different amounts of etanercept and P60 PLAD protein. (a) etanercept (10 μg) or P60 PLAD protein (100 μg) as indicated (testing protein). (b) etanercept. (0.1, 1, 10 μg) or P60 PLAD protein (0.1, 1, 10 μg) as indicated (testing protein). Arrowheads indicate the position of the TNF protein on the gel. Western blotting was carried out with antibody (Ab) against TNF-α.

FIG. 23 shows the dimerized-PLAD portion from the crystal structure of TNFR1 (PDB ID: 1NCF). Dark and light grey illustrate individual peptide chains of a PLAD dimer. The mirror imidazole rings of His-34 from each peptide are depicted within the circle. These two histidines lock up each other in an inter-chain pocket.

FIG. 24 shows immunoprecipitation (IP) and Western Blot (WB) of P80-PLAD protein mixed with etanercept.

FIG. 25 shows that P60-PLAD protein inhibited MW expression in CIA. Note that the dark (brown in its original color slide) staining in the left panel indicates MMP expression.

FIG. 26 shows that P60-PLAD protein inhibits iNOS expression in CIA. Note that the dark (brown in its original color slide) staining in the left panel indicate iNOS expression.

### DETAILED DESCRIPTION OF THE INVENTION

The present invention may be understood more readily by reference to the following detailed description of the preferred embodiments of the invention and the Examples included therein and to the Figures and their previous and following description.

As used in the specification and in the claims, “a” can mean one or more, depending upon the context in which it is used. Thus, for example, reference to “a nucleic acid” means that at least one nucleic acid is utilized.

### Polypeptides

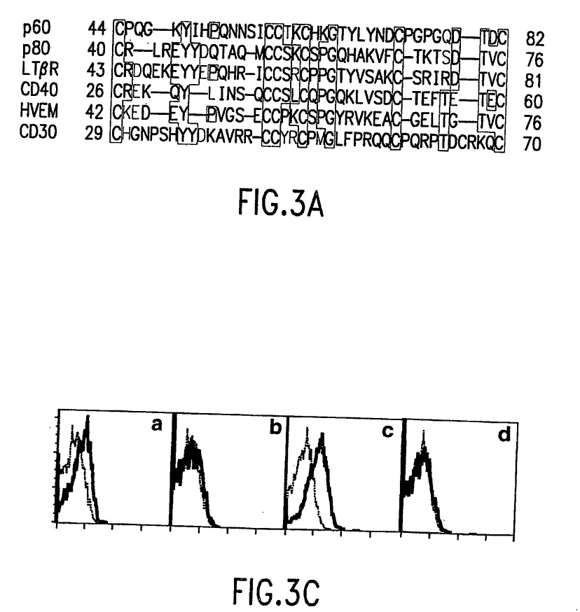

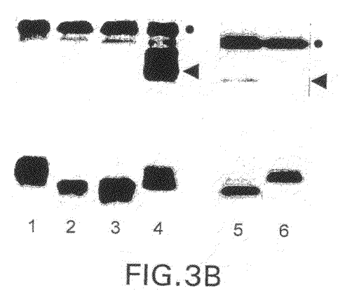

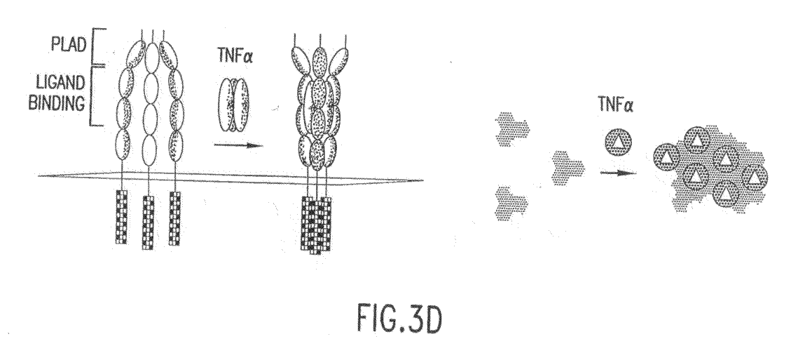

The present invention provides a polypeptide comprising the isolated amino acid sequence of a pre-ligand assembly domain (PLAD). The present invention also provides a polypeptide consisting of the amino acid sequence of a pre-ligand assembly domain. The PLAD of the present invention can be the PLAD of a TNF-R, the PLAD of p60, the PLAD of p80, the PLAD of Fas (CD95/APO-1), the PLAD of TRAIL, the PLAD of LTβR, the PLAD of CD40, the PLAD of CD30, the PLAD of CD27, the PLAD of HVEM, the PLAD of OX40, the PLAD of DR4 or any other PLAD domain from a member of the TNFR superfamily. Since the PLAD domain is highly conserved among members of the TNFR superfamily, one skilled in the art could identify the PLAD domain of any TNF receptor by searching available databases for the conserved motif that characterizes the PLAD domain. Identification of these regions in TNF receptor-like receptors is made routine by the provision of exemplary PLAD sequences herein and their comparison to published sequences of other members of the family (see FIG. 3, for example). Furthermore, one skilled in the art would also be able to identify a PLAD by performing functional assays, such as those provided in the Examples. In one embodiment the functional PLAD is not the PLAD of Fas/CD59 (83). In a further embodiment, the functional PLAD is not the amino-terminal 49 amino acids of the Fas/CD59 receptor (83).

The PLADs provided herein can comprise as few as 38 amino acids of the N-terminus of a mature TNF receptor-like receptor. A mature TNF receptor-like receptor is a TNF receptor-like receptor that does not include a signal sequence. Examples of PLADs are disclosed in the sequence listing, which includes amino acid sequences of examples of TNF receptor-like receptors including their signal sequences. The residues of the signal sequences of the respective receptors can be found by reference to the GenBank accession numbers for these TNF receptor-like receptors listed in Table 1. Thus, the sequences of the mature TNF receptor-like receptors and their corresponding PLADs are disclosed in the provided sequences. Table 3 provides additional information about the TNF receptor-like receptors and receptor ligands disclosed herein. It also provides information regarding the uses for the isolated PLADs and polypeptides containing the isolated PLADs disclosed herein. The PLADs can be used to study the implications of interfering with a signal transduction pathway mediated by a receptor of the TNFR superfamily. For example, if signaling via a receptor of the TNFR superfamily is known or shown to be associated with a disease pathway, the inhibition of receptor pre-ligand assembly by the present polypeptides, can treat or prevent the disease. For example, diseases that can be treated include cancer, heart disease and inflammatory diseases. Modifications of PLAD can also change the affinity of ligand/receptor interactions, which can be used in in vitro studies such as measuring ligand and receptor binding, receptor signals etc. Fluorescence-tagged PLAD proteins may also be utilized as reagents for determining relative expression of specific TNFRs on the surface of cells via flow cytometry or fluorescence microscopy.

The present invention also provides a polypeptide of 38 to 125 amino acids comprising an isolated PLAD. For example, the polypeptide can be from 50 to 125 amino acids comprising an isolated PLAD. In a further example, the polypeptide can comprise the subsequence R₁-TNF receptor-like receptor PLAD-R₂, wherein R₁ and R₂ are optional and when present can be H, acyl, NH₂, an amino acid or a peptide. When present, R₁ and/or R₂ can be any amino acid. When R₁ and/or R₂ is a peptide, this peptide can vary in length. For example, R₁ and/or R₂ can be 1, 2, 3, 4, 5, 6, 7, 8, 9, 10, 11, 12, 13, 14, 15, 16, 17, 18, 19, 20, 21, 22, 23, 24, 25 or more amino acids in length as long as the entire polypeptide comprising the isolated TNF-like PLAD is no more than 125 amino acid residues, and can be 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124 or 125 amino acids in length. R1 and R2 can also be sequences of the TNF receptor-like receptor that normally flank the TNF-like PLAD in a naturally occurring TNF receptor-like receptor, wherein the polypeptide comprising the TNF-like receptor PLAD is not the entire extracellular domain of a TNF receptor-like receptor.

Further provided by this invention is a polypeptide of any size, comprising the isolated amino acid sequence of a pre-ligand assembly domain (PLAD) of a TNF receptor-like receptor, wherein the polypeptide is R1-TNF receptor-like receptor PLAD-R2, wherein R1 or R2 comprise an amino acid sequence that does not flank the TNF receptor-like receptor PLAD in a naturally occurring TNF receptor-like receptor. R1 or R2, but not both can be full or partial sequences of the TNF receptor-like receptor that normally flank the TNF-like PLAD in a naturally occurring TNF receptor-like receptor. For example, the PLAD can be from a TNF receptor-like receptor and R1 or R2, can be amino acid sequences that are not present in the TNF receptor-like receptor from which the TNF-like PLAD of the polypeptide was derived or any other TNF receptor-like receptor. R1 or R2 can be any amino acid sequence as long as R1-TNF-like PLAD-R2 is not a naturally occurring full-length TNF receptor-like receptor. In another example, the PLAD can be from one TNF receptor-like receptor and R1 or R2 or both, if present, can be peptide sequences from another TNF receptor-like receptor. Therefore, one skilled in the art can combine the PLAD of one TNF receptor-like receptor with R1 or R2 sequences from a different TNF receptor-like receptor to obtain this polypeptide. Since the sequences of known TNF receptor-like receptors are publicly available, the structure of R1 and R2 of the present polypeptide are numerous but well known and contemplated herein. Alternatively, R1 or R2 can be peptide sequences that are not related to any of the TNF receptor-like receptor sequences. In one embodiment the polypeptide comprising an isolated PLAD is not the 124 amino acid sequence of the mature (lacking the signal sequence) TNF1 receptor disclosed in U.S. Pat. No. 5,633,145 (Feldman et al.) and shown in SEQ ID NO:40.

Examples of polypeptides comprising the above-mentioned subsequence include: R₁-amino acids 1-38, 1-39, 1-40, 141, 142, 143, 1-44, 1-45, 1-46, 1-47, 1-48, 1-49, 1-50, 1-51, 1-52, 1-53, or 1-54 of mature p60-R₂ (e.g., SEQ ID NO: 1); R₁-amino acids 10-48, 10-49, 10-50, 10-51, 10-52, 10-53, or 10-54 of mature p80-R₂ (SEQ ID NO: 2); R₁-amino acids 1-38, 1-39, 1-40, 1-41, 142, or 1-43 of mature Fas-R₂ (SEQ ID NO: 3); R₁-amino acids 1-38, 1-39, 1-40, 1-41, 1-42, 1-43, 1-44, 145, 1-46, 1-47, 1-48, 1-49, 1-50, 1-51, 1-52, 1-53, 1-54, 1-55, 1-56, 1-57, 1-58, 1-59, 1-60, 1-61, 1-62, 1-63, 1-64, 1-65, or 1-66 of mature Fas-R₂ (SEQ ID NO: 4); R₁-amino acids 13-50 of mature LtβR-R₂ (SEQ ID NO: 5); R₁-amino acids 6-39 of mature CD40-R₂ (SEQ ID NO: 6); R₁-amino acids 11-49, 11-50, or 11-51 of mature CD30-R₂ (SEQ ID NO: 7); R₁-amino acids 7-42 of mature CD27-R₂ (SEQ ID NO: 8), R₁-amino acids 6-37 of mature HVEM-R₂ (SEQ ID NO: 9); R₁-amino acids 3-36 of mature OX40-R₂ (SEQ ID NO: 10), and R₁-amino acids 109-138 of mature DR4-R₂ (SEQ ID NO: 11).

The mature p60 (TNFR1) polypeptide starts at position 30 of the full-length p60 coding sequence set forth as SEQ ID NO: 12. Therefore, the present invention provides a polypeptide comprising amino acids 1-54 of the mature p60 protein (amino acids 30-83 of SEQ ID NO: 12), a polypeptide comprising amino acids 1-53 of the mature p60 protein (amino acids 30-82 of SEQ ID NO: 0.12), a polypeptide comprising amino acids 1-52 of the mature p60 protein (amino acids 30-81 of SEQ ID NO: 12), a polypeptide comprising amino acids 1-51 of the mature p60 protein (amino acids 30-80 of SEQ ID NO: 12), a polypeptide comprising amino acids 1-50 of the mature p60 protein (amino acids 30-79 of SEQ ID NO: 12), a polypeptide comprising amino acids 1-49 of the mature p60 protein (amino acids 30-78 of SEQ ID NO: 12), a polypeptide comprising amino acids 1-48 of the mature p60 protein (amino acids 30-77 of SEQ ID NO: 12), a polypeptide comprising amino acids 1-47 of the mature p60 protein (amino acids 30-76 of SEQ ID NO: 12), a polypeptide comprising amino acids 1-46 of the mature p60 protein (amino acids 30-75 of SEQ ID NO: 12), a polypeptide comprising amino acids 1-45 of the mature p60 protein (amino acids 30-74 of SEQ ID NO: 12), a polypeptide comprising amino acids 1-44 of the mature p60 protein (amino acids 30-73 of SEQ ID NO: 12), a polypeptide comprising amino acids 1-43 of the mature p60 protein (amino acids 30-72 of SEQ ID NO: 12), a polypeptide comprising amino acids 1-42 of the mature p60 protein (amino acids 30-71 of SEQ ID NO: 12), a polypeptide comprising amino acids 1-41 of the mature p60 protein (amino acids 30-70 of SEQ ID NO: 12), a polypeptide comprising amino acids 1-40 of the mature p60 protein (amino acids 30-69 of SEQ ID NO: 12), and a polypeptide comprising amino acids 1-39 of the mature p60 protein (amino acids 30-68 of SEQ ID NO: 12) as well as other polypeptides comprising fragments of amino acids 1-54 of the mature p60 protein that retain PLAD activity.

The mature p80 (TNFR2) polypeptide starts at position 23 of the full-length p80 coding sequence set forth as SEQ ID NO: 13. Therefore the present invention provides a polypeptide comprising amino acids 10-54 of the mature p80 protein (amino acids 32-76 of SEQ ID NO: 13), a polypeptide comprising amino acids 10-53 of the mature p80 protein (amino acids 32-75 of SEQ ID NO: 13), a polypeptide comprising amino acids 10-52 of the mature p80 protein (amino acids 32-74 of SEQ ID NO: 13), a polypeptide comprising amino acids 10-51 of the mature p80 protein (amino acids 32-73 of SEQ ID NO: 13), a polypeptide comprising amino acids 10-50 of the mature p80 protein (amino acids 32-72 of SEQ ID NO: 13), a well as other polypeptides comprising fragments of amino acids 10-54 of the mature p80 protein that retain PLAD activity.

The mature Fas receptor polypeptide starts at position 17 of the full-length Fas coding sequence set forth as SEQ ID NO: 14. Therefore the present invention provides a polypeptide comprising amino acids 1-43 of the mature Fas protein (amino acids 17-59 of SEQ ID NO: 14), a polypeptide comprising amino acids 1-42 of the mature Fas protein (amino acids 17-58 of SEQ ID NO: 14), a polypeptide comprising amino acids 1-41 of the mature Fas protein (amino acids 17-57 of SEQ ID NO: 14), a polypeptide comprising amino acids 1-40 of the mature Fas protein (amino acids 17-56 of SEQ ID NO: 14), a polypeptide comprising amino acids 1-39 of the mature Fas protein (amino acids 17-55 of SEQ ID NO: 14), a well as other polypeptides comprising fragments of amino acids 1-43 of the mature Fas protein that retain PLAD activity.

The present invention also provides a polypeptide comprising amino acids 1-66 of the mature Fas protein (amino acids 17-82 of SEQ ID NO: 14), a polypeptide comprising amino acids 1-65 of the mature Fas protein (amino acids 17-81 of SEQ ID NO: 14), a polypeptide comprising amino acids 1-64 of the mature Fas protein (amino acids 17-80 of SEQ ID NO: 14), a polypeptide comprising amino acids 1-63 of the mature Fas protein (amino acids 17-79 of SEQ ID NO: 14), a polypeptide comprising amino acids 1-62 of the mature Fas protein (amino acids 17-78 of SEQ ID NO: 14), as well as other polypeptides comprising fragments of amino acids 1-66 of the mature Fas protein that retain PLAD activity.

The present invention also provides a polypeptide comprising amino acids 43-80 of the full-length LtβR protein set forth as SEQ ID NO: 15 as well as other polypeptides comprising fragments of amino acids 43-80 of SEQ ID NO: 15 that retain PLAD activity.

The present invention also provides a polypeptide comprising amino acids 26-59 of the full-length CD40 protein set forth as SEQ ID NO: 16 as well as other polypeptides comprising fragments of amino acids 26-59 of SEQ ID NO: 16 that retain PLAD activity.

The mature CD30 polypeptide starts at position 19 of the full-length CD30 coding sequence set forth as SEQ ID NO: 17. Therefore the present invention provides a polypeptide comprising amino acids 11-51 of the mature CD30 protein (amino acids 29-69 of SEQ ID NO: 17), a polypeptide comprising amino acids 11-50 of the mature CD30 protein (ammo acids 29-68 of SEQ ID NO: 17), a polypeptide comprising amino acids 11-49 of the mature CD30 protein (amino acids 29-67 of SEQ ID NO: 17), a polypeptide comprising amino acids 11-48 of the mature CD30 protein (amino acids 29-66 of SEQ ID NO: 17), a polypeptide comprising amino acids 11-47 of the mature CD30 protein (amino acids 29-65 of SEQ ID NO: 17), a well as other polypeptides comprising fragments of amino acids 11-51 of the mature CD30 protein that retain PLAD activity.

The present invention also provides a polypeptide comprising amino acids 27-62 of the full-length CD27 protein set forth as SEQ ID NO: 18 as well as other polypeptides comprising fragments of amino acids 27-62 of SEQ ID NO: 18 that retain PLAD activity

The present invention also provides a polypeptide comprising amino acids 42-75 of the full-length HVEM protein set forth as SEQ ID NO: 19 as well as other polypeptides comprising fragments of the polypeptide comprising amino acids 42-75 of SEQ ID NO: 19 that retain PLAD activity.

The mature OX40 polypeptide starts at position 29 of the full-length OX40 coding sequence set forth as SEQ ID NO: 20. Therefore the present invention provides a polypeptide comprising amino acids 3-36 of the mature OX40 protein (amino acids 31-64 of SEQ ID NO: 20), a polypeptide comprising amino acids 3-35 of the mature OX40 protein (amino acids 31-63 of SEQ ID NO: 20), a polypeptide comprising amino acids 3-34 of the mature OX40 protein (amino acids 31-62 of SEQ ID NO: 20), a polypeptide comprising amino acids 3-33 of the mature OX40 protein (amino acids 31-61 of SEQ ID NO: 20), a polypeptide comprising amino acids 3-32 of the mature CD30 protein (amino acids 31-60 of SEQ ID NO: 20), a well as other polypeptides comprising fragments of the polypeptide comprising amino acids 3-36 of the mature OX40 protein that retain PLAD activity.

The present invention also provides a polypeptide comprising amino acids 132-170 of the full-length DR4 protein set forth as SEQ ID NO: 21 as well as other polypeptides comprising fragments of the polypeptide comprising amino acids 132-170 of SEQ ID NO: 21 that retain PLAD activity.

Table 1 sets forth examples of TNF receptor-like receptors comprising a PLAD of the present invention. The nucleotide and polypeptide sequences for these receptors can be found under the GenBank Accession Nos. set forth in Table 1. The nucleotide sequences, the polypeptide sequences and any information (e.g., signal sequence and mature protein residue numbers) set forth under the GenBank Accession Nos. set forth in Table 1 are hereby incorporated in their entireties by this reference. For example, the nucleotide sequence, the polypeptide sequence and additional information (e.g., signal sequence and mature protein residue numbers) for p60 can be found under GenBank Accession No. M75866. These p60 sequences and additional information set forth under GenBank Accession No. M75866 are hereby incorporated in their entireties by this reference. Similarly, the nucleotide sequence, the polypeptide sequence and additional information (e.g., signal sequence and mature protein residue numbers) set forth for p80 can be found under GenBank Accession No. M32315. These p80 sequences and additional information set forth under GenBank Accession No. M32315 are hereby incorporated in their entireties by this reference. By accessing the GenBank Accession Nos. set forth in Table 1, one of skill in the art can access additional GenBank Accession Nos. listed therein to obtain additional information concerning signal sequences and mature protein sequences. For example, upon accessing GenBank Accession No. M75866, one of skill in the art can access GenBank Accession No. AAA61201 which sets forth the signal sequence and mature protein sequences information for p60. This information can also be found by directly accessing GenBank Accession Nos. AAA51201 (p60), GenBank Accession No. AAA59929 (p80), GenBank Accession No. AAA63174 (Fas), GenBank Accession No. AAA36757 (LTBR), GenBank Accession No. CAA43045 (CD40), GenBank Accession No. AAA51947 (CD30), GenBank Accession No. AAA58411 (CD27), GenBank Accession No. AAB58354, GenBank Accession No. CAA53576 (OX40), GenBank Accession No. AAC51226 (DR4), and is incorporated herein by this reference.

Table 1 also provides Locus Link Accession Nos. for the TNF-like receptors. Locus Link Accession Nos. are now equivalent to Entrez Gene Identification Numbers (Gene ID numbers) that can be accessed at the National Center for Biotechnology Information at the U.S. National Library of Medicine. For example, one of skill in the art can obtain additional information, regarding p60, including nucleotide and protein sequences, by accessing Locus Link number 7132 (now Gene D 7132 in Entrez Gene) in the Entrez Gene database. Similarly one of skill in the art can obtain additional information regarding p80, including nucleotide and protein sequences, by accessing Locus Link number 7133 (now Gene ID 7133 in Entrez Gene) in the Entrez Gene database. Thus, one of skill in the art can readily obtain information regarding any of the TNF-like receptors listed in Table 1 by accessing their respective Locus Link (Gene ID) numbers in Entrez Gene. All of the information provided under the Locus Link (Gene ID) numbers set forth in Table 1 is hereby incorporated by reference in its entirety.

Provided are polypeptides comprising the isolated amino acid sequences for PLAD domains of vTNFR proteins (SEQ ID NOS: 28-39). Other vTNFR PLADs can be identified using protein-protein BLAST database searches of homologs for TNFR1 and TNFR2 PLAD sequences. Also included are full length amino acid sequences for each vTNFR and its modified protein (SEQ. ID NOS: 44-55). Also provided is methodology for identification, production, and functional testing of and additional vTNFR PLAD polypeptides by one of skill in the art.

The vTNFR PLAD domains can disrupt self-association of host TNFRs and/or subsequent ligand binding to dampen anti-viral immunity and/or protect infected cells from TNF-mediated cell death. The M-T2 protein, a TNF-receptor like protein encoded by myxoma virus, can protect myxoma-infected T cells from TNF-induced death independently of its extracellular TNF binding capacity (82). vTNFR PLAD sequences can serve as more potent inhibitors of TNF-induced effects than P60 or P80 PLADs themselves, as a consequence of evolutionary selection for higher affinity binding to host TNFR PLAD domains. In this regard, isolated viral PLAD proteins represent improved agents for clinical use in blocking TNF-associated pathogenesis associated with rheumatoid arthritis and other autoimmune diseases.

It is understood that techniques such as those described herein can be employed to identify additional microbial proteins with homology to PLAD domains found in other TNF receptor-like receptors (e.g. Fas) described herein. For example, three examples of microbial polypeptides containing sequences homologous to the Fas PLAD domain (SEQ ID NOS: 41-43, 56-58) are disclosed.

As used herein an “isolated amino acid sequence of a PLAD” means a sequence which is substantially free from the naturally occurring materials with which the amino acid sequence is normally associated in nature. The polypeptides of this invention can comprise the entire amino acid sequence of a PLAD domain or fragments thereof that have PLAD activity. The polypeptides or fragments thereof of the present invention can be obtained by isolation and purification of the polypeptides from cells where they are produced naturally or by expression of exogenous nucleic acid encoding a PLAD. Fragments of a PLAD can be obtained by chemical synthesis of peptides, by proteolytic cleavage of the PLAD or the polypeptide comprising a PLAD and by synthesis from nucleic acid encoding the portion of interest. The PLAD can include conservative substitutions where a naturally occurring amino acid is replaced by one having similar properties. Such conservative substitutions do not alter the function of the polypeptide. Mutations that enhance binding and effectiveness can be found by creating various amino acid substitutions and testing them in binding assays described within the specification using techniques available to those of ordinary skill in the art.

Thus, it is understood that, where desired, modifications and changes can be made in the nucleic acid encoding the polypeptides of this invention and/or amino acid sequence of the polypeptides of the present invention and still obtain a polypeptide having like or otherwise desirable characteristics. Such changes can occur in natural isolates or can be synthetically introduced using site-specific mutagenesis, the procedures for which, such as mis-match polymerase chain reaction (PCR), are well known in the art.

For example, certain amino acids can be substituted for other amino acids in a polypeptide without appreciable loss of functional activity. It is thus contemplated that various changes can be made in the amino acid sequence of the PLAD (or underlying nucleic acid sequence) without appreciable loss of biological utility or activity and possibly with an increase in such utility or activity. For example, the Q24A mutation, the D49R mutation and the K19E mutation in the natural sequence of p60 TNFR do not impair PLAD self-association.

These polypeptides can also be obtained in any of a number of procedures well known in the art. One method of producing a polypeptide is to link two peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to a particular protein can be synthesized by standard chemical reactions. For example, a peptide or polypeptide can be synthesized and not cleaved from its synthesis resin whereas the other fragment of a hybrid peptide can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form a larger polypeptide. (Grant, Asynthetic Peptides: A User Guide, W.H. Freeman and Co., N.Y. (1992) and Bodansky and Trost, Ed., Principles of Peptide Synthesis, Springer-Verlag Inc., N.Y. (1993)). Alternatively, the peptide or polypeptide can be independently synthesized in vivo as described above. Once isolated, these independent peptides or polypeptides can be linked to form a larger protein via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al. Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. A Synthesis of Proteins by Native Chemical Ligation, Science, 266:776-779 (1994)). The first step is the chemoselective reaction of an unprotected synthetic peptide-%-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Clark-Lewis et al. FEBS Lett., 307:97 (1987), Clark-Lewis et al., J. Biol. Chem., 269:16075 (1994), Clark-Lewis et al. Biochemistry, 30:3128 (1991), and Rajarathnam et al. Biochemistry, 29:1689 (1994)).

Alternatively, unprotected peptide segments can be chemically linked where the bond formed between the peptide segments as a result of the chemical ligation is an unnatural (non-peptide) bond (Schnolzer et al. Science, 256:221 (1992)). This technique has been used to synthesize analogs of protein domains as well as large amounts of relatively pure proteins with full biological activity (deLisle Milton et al. ATechniques in Protein Chemistry IV, Academic Press, New York, pp. 257-267 (1992)).

The present invention also provides peptide mimetics for the disclosed polypeptides. A “peptide mimetic” is defined to include a chemical compound, or an organic molecule, or any other peptide mimetic, the structure of which is based on or derived from a binding region of a protein. For example, one can model predicted chemical structures to mimic the structure of a binding region, such as a PLAD. Such modeling can be performed using standard methods. Alternatively, peptide mimetics can also be selected from combinatorial chemical libraries in much the same way that peptides are. (Ostresh, J. M. et al., Proc Natl Acad Sci USA 1994 Nov. 8; 91(23):11138-42; Dorner, B. et al., Bioorg Med Chem 1996 May; 4(5):709-15; Eichler, J. et al., Med Res Rev 1995 November; 15(6):481-96; Blondelle, S. E. et al. Biochem J 1996 Jan. 1; 313 (Pt 1):141-7; Perez-Paya, E. et al., J Biol Chem 1996 Feb. 23; 271(8):4120-6). Functional assays can also be utilized to select peptide mimetics.

The polypeptides of this invention can be linked to another moiety such as a nucleic acid, a protein, a peptide, a ligand, a carbohydrate moiety, viral proteins, a monoclonal antibody, a polyclonal antibody or a liposome. Furthermore, two or more PLAD containing polypeptides can also be linked to each other. For example, a bifunctional or multifunctional polypeptide containing two or more different PLADs can be made such that the polypeptide is capable of modulating the activity of more than one TNF receptor-like receptor. The polypeptide can also contain two or more PLADs from the same TNF receptor-like receptor in order to increase the avidity of this polypeptide for a particular TNF receptor-like receptor.

### PLAD-Containing Fusion Constructs

Disclosed herein are fusion proteins containing PLAD and the nucleic acids encoding them. The fusion protein can comprise the PLAD of a TNF receptor-like receptor disclosed herein linked to a fusion tag. The functional molecule can be an antibody or targeting portion thereof or other fusion tag. Since PLAD is on the cell surface, the PLAD containing fusion protein can include a component that targets the PLAD to the cell surface of PLAD-expressing cells. For example, marker-binding portions of ligands for non-TNF receptor-like markers on the surfaces of intended target cells can be fused to PLAD. The PLAD can be fused to various carrier proteins such as immunoglobulin or other serum, soluble, and/or stable proteins. The fusion tag can be GST or other molecule that facilitates purification of the fusion protein. The PLAD-containing fusion protein can include a signal sequence to facilitate secretion of a recombinantly expressed PLAD.

The nucleic acids encoding a polypeptide comprising or consisting of a PLAD can also be functionally linked to other nucleic acids to encode an immunoadhesin. For the purposes of the invention, the term “immunoadhesin” is defined as including any polypeptide encoded by a nucleic acid where at least a portion of a nucleic acid encoding a non-immunoglobulin molecule such as a PLAD is coupled to at least a portion of a nucleic acid encoding an immunoglobulin heavy chain polypeptide, IgG for example. The Fc regions of IgG2, IgG3, IgM, IgA, IgE can also be utilized to construct an immunoadhesin. In a particular example, the fusion protein comprises PLAD fused to an Ig Fc portion, especially that of Ig Gamma 4. The coupling can be achieved in a manner which provides for a functional transcribing and translating of the nucleic acid segment and message derived therefrom, respectively.

The PLAD polypeptide fusion protein can be expressed by transient or stable transfection in a variety of mammalian host cells as well as in baculovirus-infected cells. The expressed fusion protein can be purified according to standard methods. Similar, to antibodies, IgG immunoadhesins can be purified from the culture medium into which they are secreted by single-step protein A or protein G affinity chromatography.

### Transgene

Provided are PLAD-encoding transgenes. By a “transgene” is meant a nucleic acid sequence that is inserted by artifice into a cell and becomes a part of the genome of that cell and its progeny. Such a transgene can be (but is not necessarily) partly or entirely heterologous (e.g., derived from a different species) to the cell. The term “transgene” broadly refers to any nucleic acid that is introduced into an animal's genome, including but not limited to genes or DNA having sequences which are perhaps not normally present in the genome, genes which are present, but not normally transcribed and translated (“expressed”) in a given genome, or any other gene or DNA which one desires to introduce into the genome. This can include genes which may normally be present in the nontransgenic genome but which one desires to have altered in: expression, or which one desires to introduce in an altered or variant form. A transgene can include one or more transcriptional regulatory sequences and any other nucleic acid, such as introns, that may be necessary for optimal expression of a selected nucleic acid. A transgene can be as few as a couple of nucleotides long, but is preferably at least about 50, 100, 150, 200, 250, 300, 350, 400, or 500 nucleotides long or even longer. A transgene can be coding or non-coding sequences, or a combination thereof. A transgene usually comprises a regulatory element that is capable of driving the expression of one or more transgenes under appropriate conditions.

### Antibodies

Also provided by the present invention are antibodies that specifically bind to a PLAD of a TNF receptor-like receptor. For example, the antibodies of the present invention can be antibodies that specifically bind to a PLAD of a TNF receptor, antibodies that specifically bind to a PLAD of FAS or antibodies that specifically bind a PLAD of DR4, to name a few. The antibody (either polyclonal or monoclonal) can be raised to any of the polypeptides provided and contemplated herein, both naturally occurring and recombinant polypeptides, and immunogenic fragments, thereof. The antibody can be used in techniques or procedures such as diagnostics, treatment, or vaccination. Anti-idiotypic antibodies and affinity matured antibodies are also considered.

Antibodies can be made by many well-known methods (See, e.g. Harlow and Lane, “Antibodies; A Laboratory Manual” Cold Spring Harbor Laboratory, Cold Spring Harbor, N.Y., (1988)). Briefly, purified antigen can be injected into an animal in an amount and in intervals sufficient to elicit an immune response. Antibodies can either be purified directly, or spleen cells can be obtained from the animal. The cells can then fused with an immortal cell line and screened for antibody secretion. The antibodies can be used to screen nucleic acid clone libraries for cells secreting the antigen. Those positive clones can then be sequenced. (See, for example, Kelly et al. Bio/Technology, 10:163-167 (1992); Bebbington et al. Bio/Technology, 10:169-175 (1992)). Humanized and chimeric antibodies are also comtemplated in this invention. Heterologous antibodies can be made by well known methods (See, for example, U.S. Pat. Nos. 5,545,806, 5,569,825, 5,625,126, 5,633,425, 5,661,016, 5,770,429, 5,789,650, and 5,814,318)

The phrase “specifically binds” with the polypeptide refers to a binding reaction which is determinative of the presence of the protein in a heterogeneous population of proteins and other biologics. Thus, under designated immunoassay conditions, the specified antibodies bound to a particular protein do not bind in a significant amount to other proteins present in the sample. Selective binding to an antibody under such conditions may require an antibody that is selected for its specificity for a particular protein. A variety of immunoassay formats can be used to select antibodies that selectively bind with a particular protein. For example, solid-phase ELISA immunoassays are routinely used to select antibodies selectively immunoreactive with a protein. See Harlow and Lane “Antibodies, A Laboratory Manual” Cold Spring Harbor Publications, New York, (1988), for a description of immunoassay formats and conditions that could be used to determine selective binding.

### Nucleic Acids

The present invention also provides nucleic acids that encode polypeptides of up to 125 amino acids comprising a PLAD of a TNF receptor-like receptor as well as nucleic acids that encode polypeptides consisting of a TNF receptor-like receptor PLAD.

The present invention also provides nucleic acids that encode a polypeptide of up to 125 amino acids comprising an isolated PLAD, wherein the polypeptide comprises the subsequence R₁-PLAD-R₂, wherein R₁ and R₂ are optional and when present can be H, acyl, NH₂, an amino acid or a peptide.

The invention further provides a nucleic acid that encodes a polypeptide comprising the isolated amino acid sequence of a pre-ligand assembly domain (PLAD) of a TNF receptor-like receptor, wherein the polypeptide is R₁-TNF receptor-like receptor PLAD-R₂, wherein R₁ or R₂ comprise an amino acid sequence that does not flank the TNF receptor-like receptor PLAD in a naturally occurring TNF receptor-like receptor.

As used herein, the term “nucleic acid” refers to single- or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the novel genes discussed herein or can include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides).

A nucleic acid molecule encoding a PLAD can be isolated from the organism in which it is normally found. For example, a genomic DNA or cDNA library can be constructed and screened for the presence of the nucleic acid of interest. Methods of constructing and screening such libraries are well known in the art and kits for performing the construction and screening steps are commercially available (for example, Stratagene Cloning Systems, La Jolla, Calif.). Once isolated, the nucleic acid can be directly cloned into an appropriate vector, or if necessary, be modified to facilitate the subsequent cloning steps. Such modification steps are routine, an example of which is the addition of oligonucleotide linkers which contain restriction sites to the termini of the nucleic acid. General methods are set forth in Sambrook et al., “Molecular Cloning, a Laboratory Manual,” Cold Spring Harbor Laboratory Press (1989). Also contemplated by the present invention are nucleic acids encoding a PLAD that do not contain a ligand binding site.

Once the nucleic acid sequence of the desired PLAD is obtained, the sequence encoding specific amino acids can be modified or changed at any particular amino acid position by techniques well known in the art. For example, PCR primers can be designed which span the amino acid position or positions and which can substitute any amino acid for another amino acid. Then a nucleic acid can be amplified and inserted into the wild-type PLAD coding sequence in order to obtain any of a number of possible combinations of amino acids at any position of the PLAD. Alternatively, one skilled in the art can introduce specific mutations at any point in a particular nucleic acid sequence through techniques for point mutagenesis. General methods are set forth in Smith, M. “In vitro mutagenesis” Ann. Rev. Gen., 19:423-462 (1985) and Zoller, M. J. “New molecular biology methods for protein engineering” Curr. Opin. Struct. Biol., 1:605-610 (1991). Techniques such as these can be used to alter the coding sequence without altering the amino acid sequence that is encoded.

Another example of a method of obtaining a DNA molecule encoding a PLAD is to synthesize a recombinant DNA molecule which encodes the PLAD. For example, oligonucleotide synthesis procedures are routine in the art and oligonucleotides coding for a particular protein region are readily obtainable through automated DNA synthesis. A nucleic acid for one strand of a double-stranded molecule can be synthesized and hybridized to its complementary strand. One can design these oligonucleotides such that the resulting double-stranded molecule has either internal restriction sites or appropriate 5′ or 3′ overhangs at the termini for cloning into an appropriate vector. Double-stranded molecules coding for relatively large proteins can readily be synthesized by first constructing several different double-stranded molecules that code for particular regions of the protein, followed by ligating these DNA molecules together. For example, Cunningham, et al., “Receptor and Antibody Epitopes in Human Growth Hormone Identified by Homolog-Scanning Mutagenesis,” Science, 243:1330-1336 (1989), have constructed a synthetic gene encoding the human growth hormone gene by first constructing overlapping and complementary synthetic oligonucleotides and ligating these fragments together. See also, Ferretti, et al., Proc. Nat. Acad. Sci. 82:599-603 (1986), wherein synthesis of a 1057 base pair synthetic bovine rhodopsin gene from synthetic oligonucleotides is disclosed. By constructing a PLAD in this manner, one skilled in the art can readily obtain any particular PLAD with desired amino acids at any particular position or positions within the PLAD. See also, U.S. Pat. No. 5,503,995 which describes an enzyme template reaction method of making synthetic genes. Techniques such as this are routine in the art and are well documented. These nucleic acids or fragments of a nucleic acid encoding a PLAD can then be expressed in vivo or in vitro as discussed below.

The invention also provides for the isolated nucleic acids encoding a PLAD in a vector suitable for expressing the nucleic acid. Once a nucleic acid encoding a particular PLAD of interest, or a region of that nucleic acid, is constructed, modified, or isolated, that nucleic acid can then be cloned into an appropriate vector, which can direct the in vivo or in vitro synthesis of that wild-type and/or modified PLAD. The vector is contemplated to have the necessary functional elements that direct and regulate transcription of the inserted gene, or nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. (See generally, Sambrook et al.).

There are numerous E. coli (Escherichia coli) expression vectors known to one of ordinary skill in the art which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences for example, for initiating and completing transcription and translation. If necessary, an amino terminal methionine can be provided by insertion of a Met codon 5′ and in-frame with the downstream nucleic acid insert. Also, the carboxy-terminal extension of the nucleic acid insert can be removed using standard oligonucleotide mutagenesis procedures.

Additionally, yeast expression can be used. There are several advantages to yeast expression systems. First, evidence exists that proteins produced in a yeast secretion systems exhibit correct disulfide pairing. Second, post-translational glycosylation is efficiently carried out by yeast secretory systems. The Saccharomyces cerevisiae pre-pro-alpha-factor leader region (encoded by the MF′-1 gene) is routinely used to direct protein secretion from yeast. (Brake, et al., Alpha-Factor-Directed Synthesis and Secretion of Mature Foreign Proteins in Saccharomyces cerevisiae. Proc. Nat. Acad. Sci., 81:4642-4646 (1984)). The leader region of pre-pro-alpha-factor contains a signal peptide and a pro-segment which includes a recognition sequence for a yeast protease encoded by the KEX2 gene: this enzyme cleaves the precursor protein on the carboxyl side of a Lys-Arg dipeptide cleavage signal sequence. The nucleic acid coding sequence can be fused in-frame to the pre-pro-alpha-factor leader region. This construct is then put under the control of a strong transcription promoter, such as the alcohol dehydrogenase I promoter or a glycolytic promoter. The nucleic acid coding sequence is followed by a translation termination codon which is followed by transcription termination signals. Alternatively, the nucleic acid coding sequences can be fused to a second protein coding sequence, such as Sj26 or β-galactosidase, used to facilitate purification of the fusion protein by affinity chromatography. The insertion of protease cleavage sites to separate the components of the fusion protein is applicable to constructs used for expression in yeast. Efficient post translational glycosylation and expression of recombinant proteins can also be achieved in Baculovirus systems.

Mammalian cells permit the expression of proteins in an environment that favors important post-translational modifications such as folding and cysteine pairing, addition of complex carbohydrate structures, and secretion of active protein. Vectors useful for the expression of active proteins in mammalian cells are characterized by insertion of the protein coding sequence between a strong viral promoter and a polyadenylation signal. The vectors can contain genes conferring hygromycin resistance, genticin or G418 resistance, or other genes or phenotypes suitable for use as selectable markers, or methotrexate resistance for gene amplification. The chimeric protein coding sequence can be introduced into a Chinese hamster ovary (CHO) cell line using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines capable of secreting intact human proteins have been developed in the art, and include the CHO cell lines, HeLa cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation can be used for other eukaryotic cellular hosts.

Alternative vectors for the expression of genes or nucleic acids in mammalian cells, those similar to those developed for the expression of human gamma-interferon, tissue plasminogen activator, clotting Factor VIII, hepatitis B virus surface antigen, protease Nexin1, and eosinophil major basic protein, can be employed. Further, the vector can include CMV promoter sequences and a polyadenylation signal available for expression of inserted nucleic acids in mammalian cells (such as COS-7).

Insect cells also permit the expression of mammalian proteins. Recombinant proteins produced in insect cells with baculovirus vectors undergo post-translational modifications similar to that of wild-type proteins. Briefly, baculovirus vectors useful for the expression of active proteins in insect cells are characterized by insertion of the protein coding sequence downstream of the Autographica californica nuclear polyhedrosis virus (AcNPV) promoter for the gene encoding polyhedrin, the major occlusion protein. Cultured insect cells such as Spodoptera frugiperda cell lines are transfected with a mixture of viral and plasmid DNAs and the viral progeny are plated. Deletion or insertional inactivation of the polyhedrin gene results in the production of occlusion negative viruses which form plaques that are distinctively different from those of wild-type occlusion positive viruses. These distinctive plaque morphologies allow visual screening for recombinant viruses in which the AcNPV gene has been replaced with a hybrid gene of choice.

The invention also provides for the vectors containing the contemplated nucleic acids in a host suitable for expressing the nucleic acids. The vectors containing the nucleic acid segments of interest can be transferred into host cells by well-known methods, which vary depending on the type of cellular host; For example, calcium chloride transformation, transduction, and electroporation are commonly utilized for prokaryotic cells, whereas calcium phosphate, DEAE dextran, or lipofection mediated transfection or electroporation can be used for other cellular hosts.

Alternatively, the nucleic acids of the present invention can be operatively linked to one or more of the functional elements that direct and regulate transcription of the inserted nucleic acid and the nucleic acid can be expressed. For example, a nucleic acid can be operatively linked to a bacterial or phage promoter and used to direct the transcription of the nucleic acid in vitro. A further example includes using a nucleic acid provided herein in a coupled transcription-translation system where the nucleic acid directs transcription and the RNA thereby produced is used as a template for translation to produce a polypeptide. One skilled in the art will appreciate that the products of these reactions can be used in many applications such as using labeled RNAs as probes and using polypeptides to generate antibodies or in a procedure where the polypeptides are being administered to a cell or a subject.

Expression of the nucleic acid, in combination with a vector, can be by either in vivo or in vitro. In vivo synthesis comprises transforming prokaryotic or eukaryotic cells that can serve as host cells for the vector. Alternatively, expression of the nucleic acid can occur in an in vitro expression system. For example, in vitro transcription systems are commercially available which are routinely used to synthesize relatively large amounts of mRNA. In such in vitro transcription systems, the nucleic acid encoding a PLAD would be cloned into an expression vector adjacent to a transcription promoter. For example, the Bluescript II cloning and expression vectors contain multiple cloning sites which are flanked by strong prokaryotic transcription promoters. (Stratagene Cloning Systems, La Jolla, Calif.). Kits are available which contain all the necessary reagents for in vitro synthesis of an RNA from a DNA template such as the Bluescript vectors. (Stratagene Cloning Systems, La Jolla, Calif.). RNA produced in vitro by a system such as this can then be translated in vitro to produce the desired PLAD polypeptide. (Stratagene Cloning Systems, La Jolla, Calif.).

### Gene Therapy Methods

Using gene therapy methods, a nucleic acid encoding a polypeptide comprising or consisting of a PLAD can be administered. The nucleic acid encoding the polypeptide of this invention can be placed into a vector and delivered to the cells of a subject either in vivo or ex vivo by standard methods.

The nucleic acid encoding the polypeptide of this invention can be functionally attached to a specific leader peptide which can specify for secretion of the polypeptide. For example the polypeptide can have a signal sequence, such as the murine Ig-kappa signal sequence (Blezinger et al. Nat. Biotechnol. 17: 343-8, 1999), rat insulin leader sequence (Fakhral et al. J. Immunother. 20: 437-8, 1997), FGF-4 signal sequence (Ueno et al. Aterioscler. Thromb. Vasc. Biol., 17: 2453-2460, 1997), human growth hormone signal peptide (Rade et al. Gene Ther. 6: 385-92, 1999), beta lactamase signal sequence (Hughes et al. Hum. Gene Ther. 5: 1445-55, 1994), bovine prolactin signal sequence (Gorman et al. Bran Res. Mol. Brain. Res. 44:143-146, 1997) and other similar signal sequences.

For in vivo administration, the cells can be in a subject and the nucleic acid can be administered in a pharmaceutically acceptable carrier. The subject can be any animal in which it is desirable to selectively express a nucleic acid in a cell. In a preferred embodiment, the animal of the present invention is a human. In addition, non-human animals which can be treated by the method of this invention can include, but are not limited to, cats, dogs, birds, horses, cows, goats, sheep, guinea pigs, hamsters, gerbils and rabbits, as well as any other animal in which selective expression of a nucleic acid in a cell can be carried out according to the methods described herein.

In the method described above which includes the introduction of exogenous DNA into the cells of a subject (i.e., gene transduction or transfection), the nucleic acids of the present invention can be in the form of naked DNA or the nucleic acids can be in a vector for delivering the nucleic acids to the cells for expression of the nucleic acid inside the cell. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as Lipofectin®, Lipofectamine® (GIBCO-BRL, Inc., Gaithersburg, Md.), Superfect® (Qiagen, Inc. Hilden, Germany) and Transfectam® (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a Sonoporation machine (ImaRx Pharmaceutical Corp., Tucson, Ariz.).

As one example, vector delivery can be via a viral system, such as a retroviral vector system which can package a recombinant retroviral genome. The recombinant retrovirus can then be used to infect and thereby deliver nucleic acid to the infected cells. The exact method of introducing the nucleic acid into mammalian cells is, of course, not limited to the use of retroviral vectors. Other techniques are widely available for this procedure including the use of adenoviral vectors, adeno-associated viral (AAV) vectors, lentiviral vectors, pseudotyped retroviral vectors, and pox virus vectors, such as vaccinia virus vectors. Physical transduction techniques can also be used, such as liposome delivery and receptor-mediated and other endocytosis mechanism. This invention can be used in conjunction with any of these or other commonly used gene transfer methods.

The nucleic acid and the nucleic acid delivery vehicles of this invention, (e.g., viruses; liposomes, plasmids, vectors) can be in a pharmaceutically acceptable carrier for in vivo administration to a subject. By “pharmaceutically acceptable” is meant a material that is not biologically or otherwise undesirable, i.e., the material can be administered to a subject, along with the nucleic acid or vehicle, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The nucleic acid or vehicle can be administered orally, parenterally (e.g., intravenously), by intramuscular injection, by intraperitoneal injection, transdermally, extracorporeally, topically or the like. The exact amount of the nucleic acid or vector required will vary from subject to subject, depending on the species, age, weight and general condition of the subject, the severity or mechanism of any disorder being treated, the particular nucleic acid or vehicle used, its mode of administration and the like.

### Inhibitors of PLAD Self-Association

The present invention further provides a composition comprising an inhibitor of PLAD self-association or TNF-like receptor oligomerization. An “inhibitor” is defined as a compound that binds a PLAD or a compound, including antibodies, that binds the target for a PLAD and prevents an activity of a PLAD. Upon binding to a PLAD, the inhibitor can disrupt or prevent PLAD self-association, thus inhibiting TNF receptor-like receptor oligomerization. The inhibitor of TNF-like receptor oligomerization can be an antibody, either polyclonal or monoclonal, that specifically binds to a PLAD, a ligand that binds to a PLAD, a polypeptide that binds to a PLAD, a compound that binds to a PLAD or a peptide mimetic based on a PLAD. For example, a polypeptide comprising or consisting of a PLAD can associate with the PLAD of a naturally occurring TNF receptor-like receptor, thus preventing or inhibiting the TNF receptor-like receptor from self-associating with other naturally occurring TNF receptor-like receptors. The polypeptide comprising or consisting of a PLAD can be a soluble PLAD. Anti-idiotypic antibodies and affinity matured antibodies are also considered. Other inhibitors include, but are not limited to molecules or compounds designed to block PLAD self-association. The inhibitor can be a whole protein or a fragment of a protein that inhibits PLAD self-association, thus preventing TNF receptor-like receptor oligomerization. The inhibitor can be an organic molecule identified according to the methods described herein. Crystal structures of the TNF receptors and their oligomeric complexes can be utilized to design molecules that can disrupt PLAD self-association. The crystal structures can also be analyzed to design molecules that mimic PLAD and disrupt PLAD self-association.

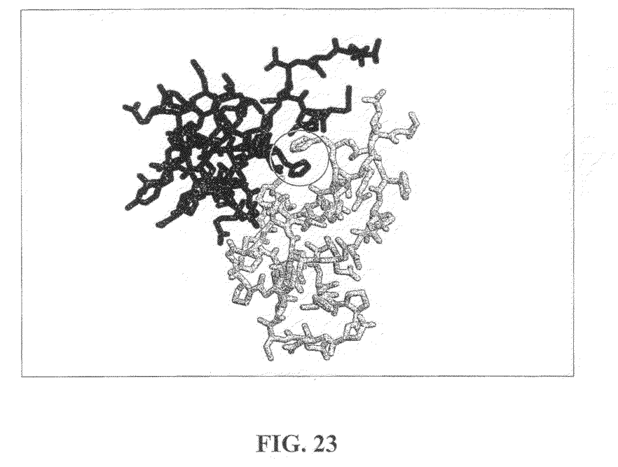

Thus, a method of making a small molecule inhibitor is provided, as is the inhibitor produced by this method. Provided are small molecules (SM) and PLAD-peptide derivatives that interfere with TNFR assembly and TNF binding. Based on the crystal structure of dimerized TNFR1 protein, a search for the binding surfaces of PLAD association was performed. This in combination with a conservative homology search and data on PLAD mutagenesis described herein, allows the identification of some potential inter-chain association sites. For example, as illustrated in the dimerized PLAD structure (FIG. 23), the two histidine rings at position 34 from each peptide chain seem to mirror-lock each other within an inter-chain pocket. Since imidazoles of histidine residues are good candidates for disruption of PLAD self-association, imidazole and imidazole derivatives can be potent receptor-specific blockers and be used to treat TNF-mediated diseases.

Disclosed herein are compounds that can interfere with TNFR assembly and TNF binding. Generally, suitable inhibitors are those compounds that can enter the inter-chain pocket of the PLAD dimerized structure and interfere with the interaction between the two histidine rings at position 34. In this sense, inhibitors containing bulky substituents that hinder entry into the inter-chain pocket or prevent the disruption of the interaction between the two histidine residues at position 34 are not preferred. Conversely, inhibitors that contain substituents that allow easy entry into the inter-chain pocket and facilitate insertion and consequent disruption of the interaction between the two histidine residues at position 34 are preferred. Further, inhibitors containing substituents that have increased affinity for other residues in the inter-chain pocket, thus facilitating and/or enhancing binding of the inhibitor in the PLAD dimerized structure are even more preferred. Analyzing putative inhibitors to evaluate whether certain substituents enhance or hinder binding to the PLAD dimerized structure can be performed in silico by those of skill in the art.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OA¹ where A¹ is alkyl as defined above.

The term alkoxylalkyl as used herein is an alkyl group that contains an alkoxy substituent and can be defined as -A¹-O-A², where A¹ and A² are all groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻.

The term “ester” as used herein is represented by the formula —OC(O)A¹ or —C(O)OA¹, where A¹ can be an alkyl, halogenated alkyl alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiA¹A²A³, where A¹, A², and A³ can be, independently, hydrogen, alkyl, halogenated alkyl, alkoxy, alkenyl alkynyl, aryl, heteroaryl, cycloalkyl, cycloalkenyl, heterocycloalkyl, or heterocycloalkenyl group described above.

The term “sulfo-oxo” as used herein is represented by the formulas —S(O)A¹, —S(O)₂A¹, —OS(O)₂A¹, or —OS(O)₂OA¹, where A¹ can be hydrogen, an alkyl, halogenated alkyl, alkenyl, alkyl, aryl, heteroaryl, cycloalkyl cycloalkenyl, heterocycloalkyl, or heterocycloalkenyl group described above. Throughout this specification “S(O)” is a short hand notation for S═O

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “sulfone” as used herein is represented by the formula A¹S(O)₂A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “sulfoxide” as used herein is represented by the formula A¹S(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

Provided is a method of inhibiting ligand binding to a TNF receptor-like receptor by administering an effective amount of an imidazole or imidazole derivative.

Examples of inhibitors that are contemplated herein are generally 5-membered nitrogen containing heterocycles functionalized with various substituents. Examples of such inhibitors are shown below and generically identified by the name of the basic unsubstituted heterocylic structure (i.e., where Rⁿ is H).

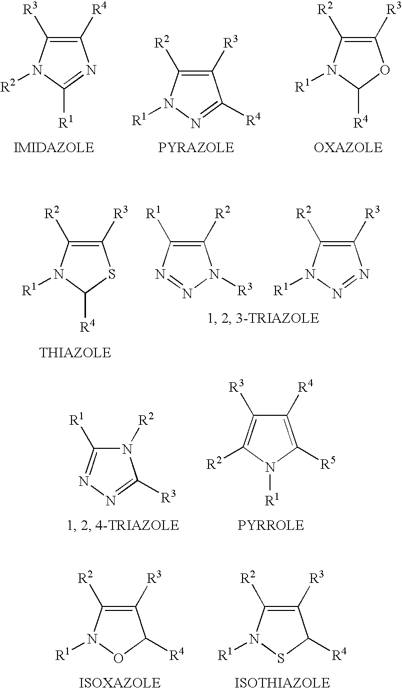

In many examples of inhibitors having the structures shown above, R¹, R², R³, R⁴, and R⁵, when present, are independently H, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol. In specific examples, R¹⁻⁵ are independently a C₁-C₆ alkyl, a C₁-C₆ alkoxyalkyl group, or a C₁-C₆ alkoxy group. Exemplary C₁-C₆ alkyl groups and C₁-C₄ alkyl groups include methyl, ethyl, propyl, iso-propyl, butyl, sec-butyl, iso-butyl, pentyl, iso-pentyl, hexyl, 2-ethylbutyl, 2-methylpentyl, and the like. Corresponding C₁-C₆ alkoxy groups contain the above C₁-C₆ alkyl group bonded to an oxygen atom that is also bonded to the cation ring. An alkoxyalkyl group contains an ether group bonded to an alkyl group, and here contains a total of up to six carbon atoms. It is to be noted that there are two isomeric 1,2,3-triazoles.

The invention also contemplates targeting other regions of TNF receptor-like receptors such that upon binding that region, the conformation of a PLAD in the receptor is disrupted thus preventing it from associating with another PLAD. For example, one skilled in the art could target the CRD3 of p60 TNFR, such that upon binding the CRD3 region of p60 TNFR, the conformation of the receptor is changed, thus preventing the PLAD of the p60 TNFR from associating with another PLAD.

Thus, further provided by the present invention is a method of inhibiting TNF receptor-like receptor oligomerization in a cell by administering an effective amount of an inhibitor of TNF receptor-like receptor oligomerization.

The invention also contemplates enhancing PLAD self-association in order to enhance the effects of a TNF receptor-like receptor. For example, there are circumstances in which it would be desirous to enhance TNFR signaling. In such instances, agonists of PLAD self association, such as certain antibodies or molecules that bind to a PLAD and have the specific property of enhancing PLAD self association, can be utilized to convert cells that are resistant to TNFR effects due to weak PLAD interactions, into cells that are responsive to TNFR effects. Such enhanced PLAD self association can increase ligand binding as well as signaling. Examples of disease states where such enhanced PLAD interactions would be desirable include, but are not limited to, autoimmune lymphoproliferative syndrome (ALPS) and hyper IgM syndrome.

The invention also provides for utilizing a PLAD as a targeting moiety to deliver biological agents to cells. For example, a PLAD linked to a toxin can be delivered to cells, such that upon binding to a naturally occurring PLAD on a TNF-R, oligomerization is inhibited and upon internalization of the naturally occurring TNF-R, the PLAD linked to the toxin is internalized as well, thus delivering the toxin to the cell.

As used throughout, “TNF receptor-like receptor” refers to any member of the TNF receptor superfamily that includes, but is not limited to: TNF-R, p60 (also known as p55 and TNFR1), p80 (also known as p75, TNFR2), Fas (CD95/APO-1), TRAIL receptor, LTβR, CD40, CD30, CD27, HVEM, OX40, DR4, TROY, EDAR, XEDAR, DCR3, AITR, 4-1BB, DR3, RANK, TACT BCMA, DR6, DPG, DR5, DCR1 AND DCR2 (See Table 1). The nucleotide sequences, the polypeptide sequences and any information (e.g., signal sequence and mature protein residue numbers) set forth under the provided GenBank Accession Nos. set forth in Table 1 are hereby incorporated in their entireties by this reference.

As previously stated, inhibitors of TNF receptor-like receptor oligomerization include antibodies, ligands, peptide mimetics, compounds and polypeptides that specifically bind to a PLAD. These polypeptides include polypeptides comprising or consisting of an isolated (e.g., soluble) PLAD.

The present invention also provides a method of inhibiting ligand binding to a TNF receptor-like receptor by administering an effective amount of an inhibitor of TNF receptor-like receptor oligomerization. For example, by administering an inhibitor, such as a polypeptide comprising or consisting of a TNFR-PLAD, TNF receptor oligomerization would be inhibited, thus preventing the binding of TNF-α to the TNF receptor and diminishing the deleterious effects of TNF-α Similarly, the administration of a polypeptide comprising a CD40 receptor-PLAD (CD40R-PLAD), would inhibit CD40R oligomerization, thus preventing the binding of CD40 ligand to the CD40R and diminishing the deleterious effects of CD40 in disease states such as allograft rejection, rheumatoid arthritis and systemic lupus erythematosis. Inhibition of ligand binding to a TNF receptor-like receptor results in inhibition of signal transduction via TNF receptor-like receptors, thus providing a method of modulating signaling via TNF receptor-like receptors. Furthermore, the present invention has established that TNF receptor-like receptors bind ligand and signal via homotypic association, i.e. TNFR-PLAD interacts with TNFR-PLAD; Fas-PLAD interacts with Fas-PLAD; CD40-PLAD interacts with CD40-PLAD etc. Therefore, therapy with PLAD self-association disrupting peptides and peptide mimetics would ensure receptor specific therapy because the present invention shows that each receptor associates only with itself through the PLAD. For example, disrupting TNF-R1 function without affecting TNF-R2 has major benefits above current non-selective therapeutics. Similarly, the specific disruption of a particular TNF receptor-like receptor function without affecting other TNF receptor-like receptor functions is highly desirable and provided by the teaching herein.

### Protein Therapy Methods

The present invention also provides a method of treating inflammation in a subject by administering an effective amount of an inhibitor of PLAD self-association. The present invention also provides a method of treating inflammation associated with an autoimmune disease in a subject by administering an effective amount of an inhibitor of PLAD self-association. Such diseases include, but are not limited to, periodic fever syndromes, sepsis syndromes and adult respiratory distress syndrome. Thus, provided is a method wherein the inflammation is associated with septic arthritis and the inhibitor is a soluble PLAD of a TNF receptor-like receptor.

In the present invention, the subject can be any mammal, preferably human, and can include but is not limited to mouse, rat, guinea pig, hamster, rabbit, cat, dog, goat, monkey, horse and chimpanzee.

As used herein, “treating” describes an improvement in the patient's clinical state. The improvement may range from reduction of the inflammatory response to complete amelioration of the inflammatory disease.

As used herein, “autoimmune disease” describes a disease state or syndrome whereby a subject's body produces a dysfunctional immune response against the subject's own body components, with adverse effects. This may include production of B cells which produce antibodies with specificity for all antigens, allergens or major histocompatibility (MHC) antigens, or it may include production of T cells bearing receptors that recognize self-components and produce cytokines that cause inflammation. Examples of autoimmune diseases include, but are not limited to, ulcerative colitis, Crohn's disease, multiple sclerosis, rheumatoid arthritis, septic arthritis, diabetes mellitus, pernicious anemia, autoimmune gastritis, psoriasis, Bechet's disease, Wegener's granulomatosis, Sarcoidois, autoimmune thyroiditis, autoimmune oophoritis, bullous pemphigoid, phemphigus, polyendocrinopathies, Still's disease, Lambert-Eaton myasthenia syndrome, myasthenia gravis, Goodpasture's syndrome, autoimmune orchitis, autoimmune uveitis, systemic lupus erythematosus, Sjogren's Syndrome and ankylosing spondylitis.

Since certain TNFR receptors, such as HVEA, are viral receptors, and these receptors may depend on oligomerization, the present invention also contemplates blocking viral entry by preventing PLAD assembly.

Optimal dosages used will vary according to the individual being treated and the inhibitor being used. The amount of inhibitor will also vary among individuals on the basis of age, size, weight, condition, etc. One skilled in the art will realize that dosages are best optimized by the practicing physician and methods for determining dose amounts and regimens and preparing dosage forms are described, for example, in Remington's Pharmaceutical Sciences. For example, suitable doses and dosage regimens can be determined by comparison to agents presently used in the treatment or prevention of inflammation or autoimmune disorders.

Typically, the inhibitor of this invention can be administered orally or parenterally in a dosage range of 0.1 to 100 mg/kg of body weight depending on the clinical response that is to be obtained. For example, when the inhibitor is a soluble PLAD or PLAD-containing compound, the inhibitor can be administered at an amount of 0.5 to 100 mg/kg, for example 5 mg/kg. In a further example, when the inhibitor is soluble (isolated) p60 PLAD and the disease is arthritis (e.g., septic arthritis), the PLAD can be administered in a dosage or 0.5 to 100 mg per joint. For example, doses of p60 PLAD of 4 mg/kg for intra-articular administration or 16 mg/kg for parenteral administration are effective to treat septic arthritis. Administration of inhibitor can be stopped completely following a prolonged remission or stabilization of disease signs and symptoms and readministered following a worsening of either the signs or symptoms of the disease, or following a significant change in immune status, as determined by routine follow-up immunological studies well known to a clinician in this field.

The efficacy of administration of a particular dose of inhibitor in treating inflammation or an autoimmune disorder as described herein can be determined by evaluating the particular aspects of the medical history, the signs, symptoms and objective laboratory tests that have a documented utility in evaluating pathophysiological activity of the particular disorder being treated. These signs, symptoms and objective laboratory tests will vary depending on the particular disorder being treated, as will be well known to any clinician in this field. For example, if, based on a comparison with an appropriate control group and knowledge of the normal progression of the disorder in the general population or the particular individual, 1) a subject's frequency or severity of recurrences is shown to be improved; 2) the progression of the disease or disorder is shown to be stabilized; or 3) the need for use of other immunosuppressive medications is lessened, then a particular treatment can be considered efficacious.

Once it is established that disease activity is significantly improved or stabilized by a particular inhibitor, specific signs, symptoms and laboratory tests can be evaluated in accordance with a reduced or discontinued treatment schedule. If a disease activity recurs, based on standard methods of evaluation of the particular signs, symptoms and objective laboratory tests as described herein, treatment can be reinitiated.

Additionally, the efficacy of administration of a particular dose of a peptide ligand in preventing an autoimmune disorder in a subject not known to have an autoimmune disorder, but known to be at risk of developing an autoimmune disorder, can be determined by evaluating standard signs, symptoms and objective laboratory tests, known to one of skill in the art, over time. This time interval may be long (i.e., years/decades). The determination of who would be at risk for the development of an autoimmune disorder would be made based on current knowledge of the known risk factors for a particular disorder familiar to clinicians and researchers in this field, such as a particularly strong family history of a disorder or exposure to or acquisition of factors or conditions which are likely to lead to development of an autoimmune disorder.

By “pharmaceutically acceptable” is meant a material that is not biologically or otherwise undesirable, i.e., the material can be administered to an individual along with the selected compound without causing any undesirable biological effects or interacting in a undesirable manner with any of the other components of the pharmaceutical composition in which it is contained. The carrier chosen depends on the method of administration and the particular patient. Methods of administration can be oral, sublingual, mucosal, inhaled, absorbed, or by injection. It is also noted that not all methods of administering the inhibitors of TNF receptor-like receptor oligomerization described herein require a pharmaceutically acceptable carrier.

In the present invention, the inhibitors of PLAD self-association or TNF-like oligomerization can be orally or parenterally administered in a carrier pharmaceutically acceptable to human subjects. Suitable carriers for oral or inhaled administration can include one or more of the carriers pharmaceutically acceptable to human subjects. Suitable carriers for oral administration include one or more substances which may also act as flavoring agents, lubricants, suspending agents, or as protectants. Suitable solid carriers include calcium phosphate, calcium carbonate, magnesium stearate, sugars, starch, gelatin, cellulose, carboxypolymethylene, or cyclodextrans. Suitable liquid carriers include water, pyrogen free saline, pharmaceutically accepted oils, or a mixture of any of these. The liquid can also contain other suitable pharmaceutical addition such as buffers, preservatives, flavoring agents, viscosity or osmo-regulators, stabilizers or suspending agents. Examples of suitable liquid carriers include water with or without various additives, including carboxypolymethylene as a ph-regulated gel. The inhibitor can be contained in enteric coated capsules that release the polypeptide into the intestine to avoid gastric breakdown. For parenteral administration of the antagonist, a sterile solution or suspension is prepared in saline that can contain additives, such as ethyl oleate or isopropyl myristate, and can be injected for example, into subcutaneous or intramuscular tissues, as well as intravenously.

### Screening Methods

A method of screening for an inhibitor of PLAD association comprising: a) transfecting a cell with a plasmid containing a nucleic acid comprising a nucleic acid sequence encoding an isolated PLAD and a plasmid comprising a nucleic acid sequence encoding a second isolated PLAD; b) contacting the cell with a putative inhibitor and; c) measuring PLAD self association, wherein a decrease in PLAD association in the cell of step b) as compared to PLAD association in a cell that was not contacted with the putative inhibitor indicates the presence of an inhibitor of PLAD-association.

One example of this screening method is a method of screening for an inhibitor of PLAD-association comprising: a) transfecting a cell with a plasmid containing a nucleic acid comprising a nucleic acid sequence encoding an isolated PLAD functionally linked to a flourescence donor and a plasmid comprising a nucleic acid sequence encoding an isolated PLAD functionally linked to a flourescence acceptor; b) contacting the cell with the inhibitor; and c) measuring FRET, wherein a decrease in FRET as compared to FRET measurement in a cell that was not contacted with the inhibitor indicates the presence of an inhibitor of PLAD-association.

Also provided by the present invention is a method of screening for an agonist of PLAD association comprising: a) transfecting a cell with a plasmid containing a nucleic acid comprising a nucleic acid sequence encoding an isolated PLAD and a plasmid comprising a nucleic acid sequence encoding a second isolated PLAD; b) contacting the cell with a putative agonist and; c) measuring PLAD self association, wherein an increase in PLAD association in the cell of step b) as compared to PLAD association in a cell that was not contacted with the putative agonist indicates the presence of an agonist of PLAD-association.

The Examples below exemplify the use of FRET to measure PLAD association. Furthermore, in performing the screening methods described above, a single plasmid can be utilized to deliver more than one nucleic acid encoding a PLAD. In methods involving FRET analysis, a single plasmid can be utilized to deliver more than one nucleic acid encoding a PLAD functionally linked to a fluorescence donor or acceptor.

One skilled in the art could also utilize a yeast two hybrid screening method to screen for inhibitors or agonists of PLAD association. Inhibitors or agonists of PLAD association can also be identified by utilizing cellular assays which can include, but are not limited to, apoptosis induction, NF-KB induction, lymphocyte maturation or activation, and necrosis induction (3, 4, 8, 15, 29, 33, 42, 45).

The present invention is more particularly described in the following examples which are intended as illustrative only since numerous modifications and variations therein will be apparent to those skilled in the art.

### Example I

H9 lymphoma cells were washed and resuspended in PBS. The cells were then incubated with 100 ng/ml of human recombinant TNFα (R&D Systems) for 1 hour at 4° C. with rotation. Cells were then treated with 2 mM of the crosslinker DTSSP (Pierce) for 30 minutes and the reaction was quenched with 20 mM Tris.Cl [pH 7.5] for 15 minutes on ice. The cells were lysed in 150 mM NaCl, 20 M Tris.Cl [pH 7.5], 1 μM EDTA, 30 mM NaF, 2 mM b-glycerophosphate and 1 mM sodium orthovanadate with protease inhibitors added (Boehringer Mannheim). Equal amounts of the lysates were subjected to electrophoresis under non-reducing (without β-mercaptoethanol) or reducing (with 280 mM β-mercaptoethanol) conditions and analyzed for p60 and p80 complexes with specific antibodies (19). Densitometry was performed with a Kodak Image Station 440.

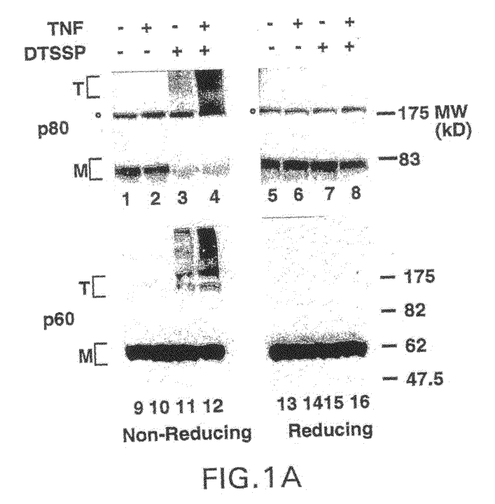

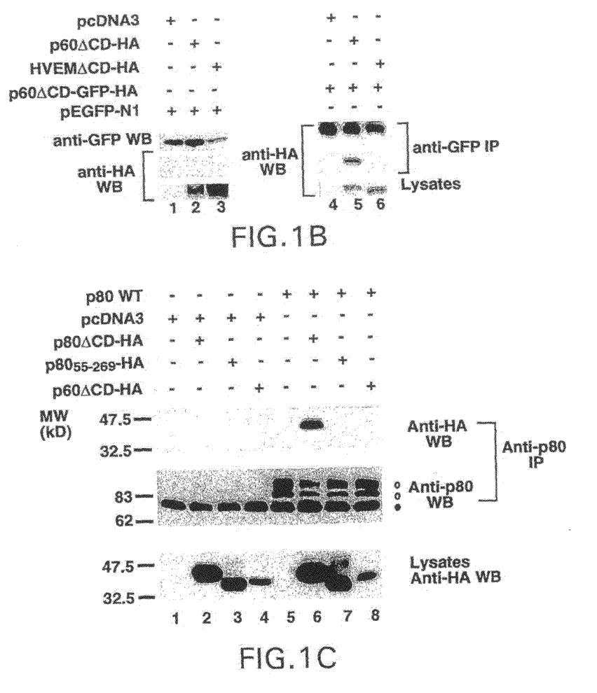

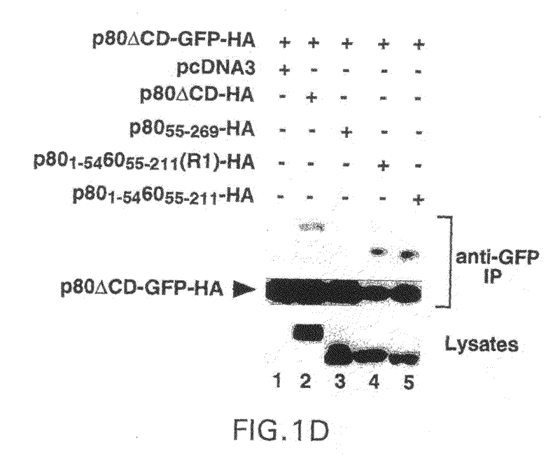

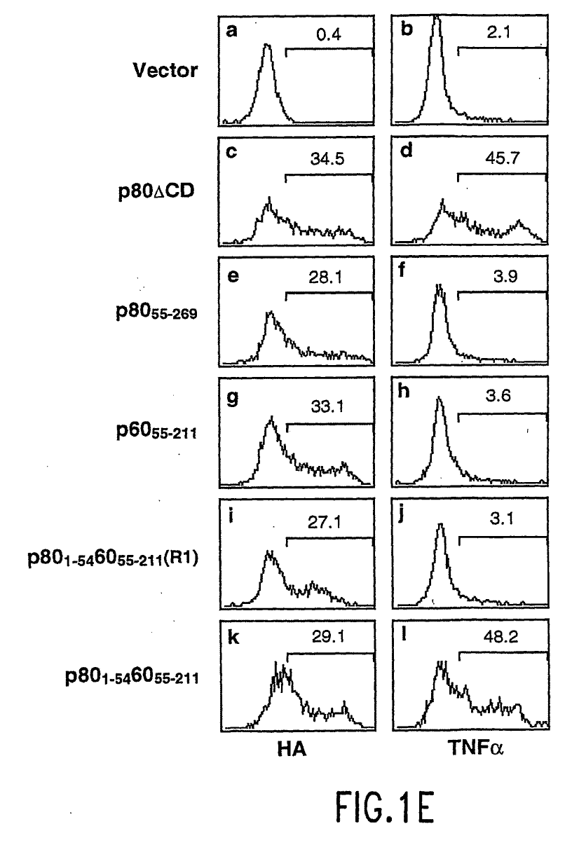

Complexes were found for p80 that exhibited molecular sizes approximately three times the unit size, consistent with glycosylated and non-glycosylated trimers (FIG. 1A). Surprisingly, p80 complexes were efficiently captured in the presence or absence of TNFα (65-70% of chains by densitometry, FIG. 1A, lanes 3 and 4). Despite the fact that most p60 resides in the Golgi apparatus and was inaccessible to the crosslinker (7), as much as 15-20% of the p60 chains were cross-linked as apparent trimers and discrete higher order complexes, whether or not TNFα was added (FIG. 1A, lanes 11 and 12). Control experiments revealed no detectable endogenous TNFα and no evidence of other proteins such as p80 crosslinked to the p60 complex. Western blot analysis confirmed the absence of TNFα in the lysates. Immunoprecipitation of the crosslinked complexes with anti-p60 antibody revealed no detectable level of p80 in the p60 complex in Western blots.

The complexes were resolved into monomers by cleaving the crosslinker with β-mercaptoethanol (FIG. 1A, lanes 7, 8, 15 and 16). Thus, these results are suggestive of p60 and p80 chain self-association prior to ligand binding.

To validate the possibility of ligand-independent self-assembly, a domain in the TNFR that would mediate this phenomenon was identified. It is well established that the cytoplasmic death domain of p60 can self-associate and trigger apoptosis when over-expressed (8). However, since the pre-assembled complexes observed were apparently non-signaling, it was hypothesized that the assembly domain resides outside of the cytoplasmic region. It was found that the N-terminal regions of the ECDs of p60 and p80 could specifically self-associate in a yeast two-hybrid interaction assay (9).

The various truncations and mutations of p60, p80, HVEM, DR4 and CD40 were generated by Polymerase chain reaction (PCR) and sequenced. Briefly, the leader sequence and the first ten amino acid residues from p80 was amplified so that the HA epitope tag was included at the 3′ end to create a HA tag at the N-terminus of the receptors. The PCR product was digested with BamHI and EcoRI and cloned into pcDNA3. The PCR fragments containing the receptor fragments were then introduced into this plasmid using the EcoRI and XhoI sites. For the GFP/CFP/YFP chimeras, the fragments were amplified by PCR and introduced in-frame into the XhoI and XbaI sites of p60ΔCD-HA. 293T cells were transfected with Fugene 6 (Boehringer Mannheim) as per manufacturer's protocol. Cells were lysed in 150 mM NaCl, 20 mM Tris.Cl [pH 7.5], 1 mM EDTA, 30 mM NaF, 2 mM β-glycerophosphate, 1 mM sodium orthovanadate, 5 mM iodoacetamide, 2 mM dithiothreitol (DTT), 1% TRITON X-100 and protease inhibitors (Boehringer Mannheim). After pre-clearing with protein G agarose beads (Boehringer Mannheim) and normal mouse IgG, proteins were immunoprecipitated from the lysates with 2 mg anti-GFP and protein G agarose beads. Immune complexes were washed twice with lysis buffer containing 0.5 M NaCl and then three times with regular lysis buffer. Immune-complexes were resolved on Tris/Glycine gels (Novex). Transfection in Jurkat cells showed similar results. In mammalian cells, it was found that a chimeric p60 receptor with the cytoplasmic domain replaced by the green fluorescent protein (GFP) interacted strongly with a tailless p60 (p60ΔCD-HA) but not with the TNFR-like receptor herpesvirus entry mediator (HVEMΔCD-HA) (FIG. 1B, compare lanes 2 and 3).

GFP alone failed to associate with p60ΔCD-HA (FIG. 1B, lane 5). A similar homotypic interaction of the extracellular portion was observed between full-length p80 and a tailless p80 (FIG. 1C, lane 6).

Moreover, removal of as little as amino acids (a.a)10-54 of p80, overlapping CRD1, completely abrogated ligand-independent association with intact p80 (FIG. 1C, lane 7). Self-association was eliminated by a similar deletion (a.a.1-54) in p60 (see below).

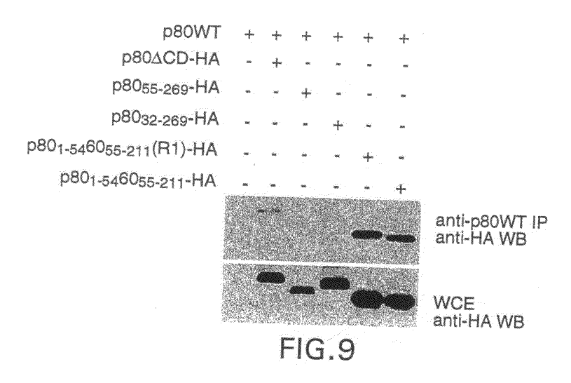

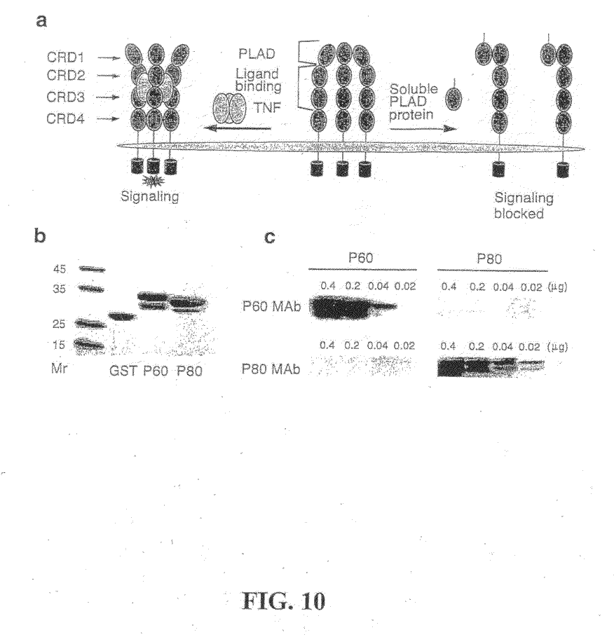

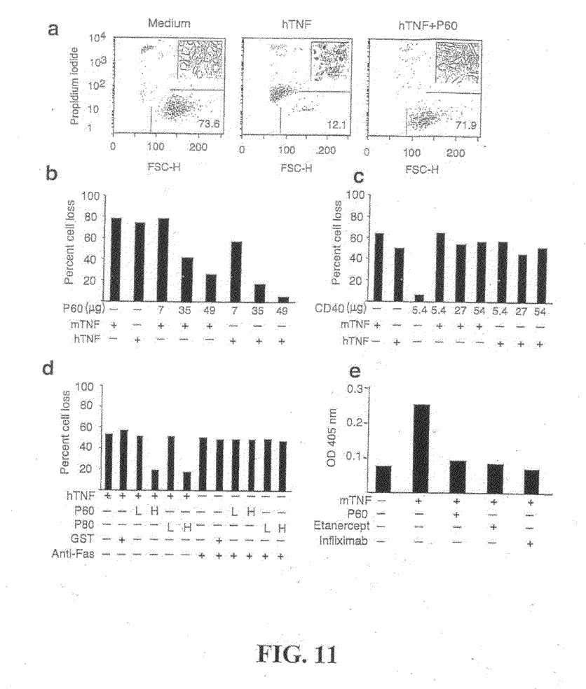

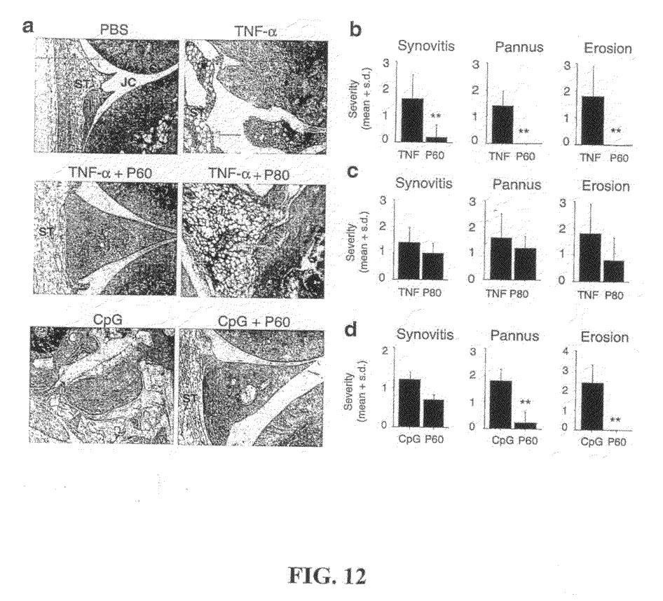

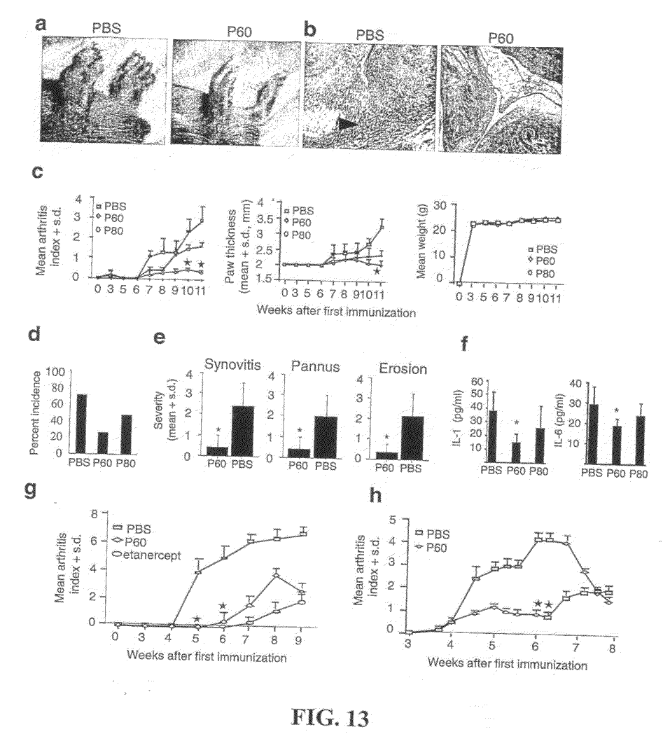

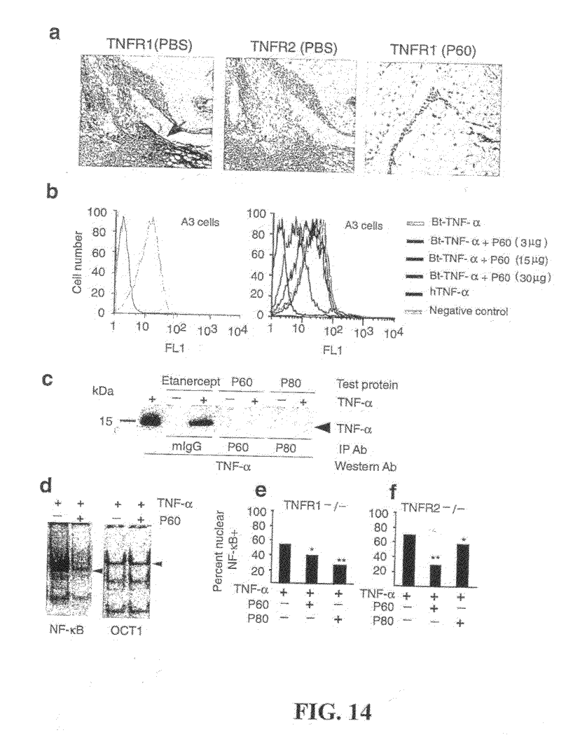

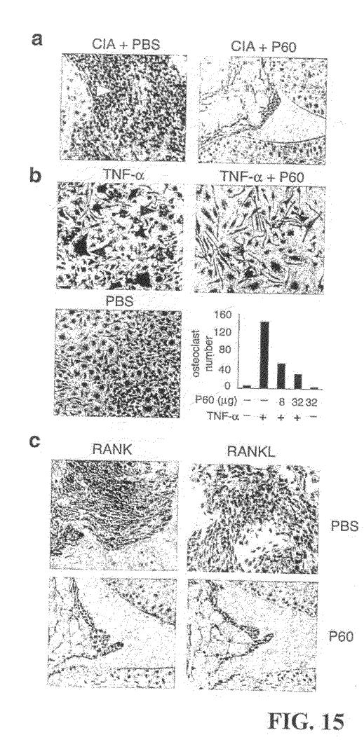

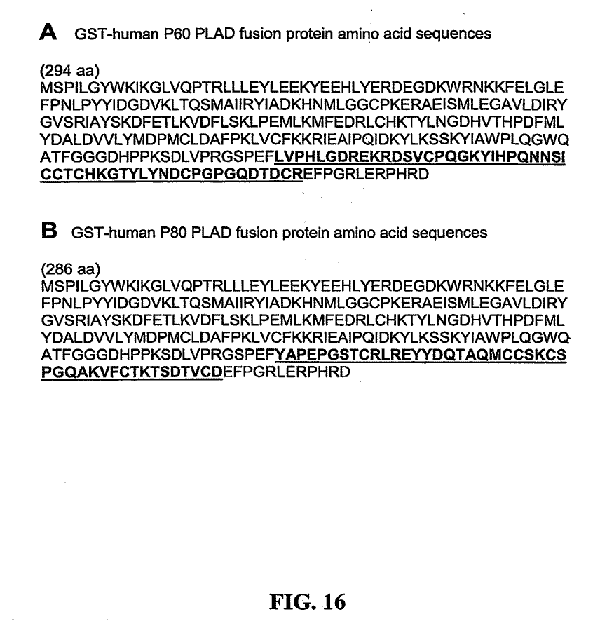

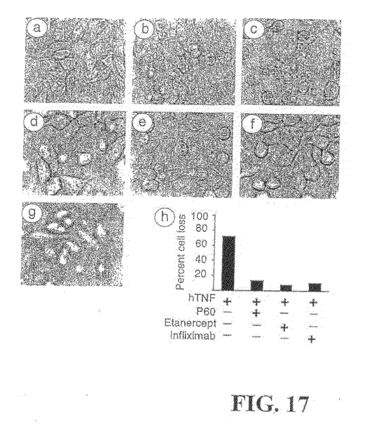

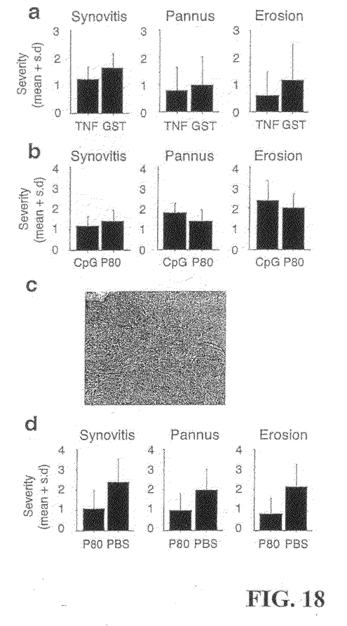

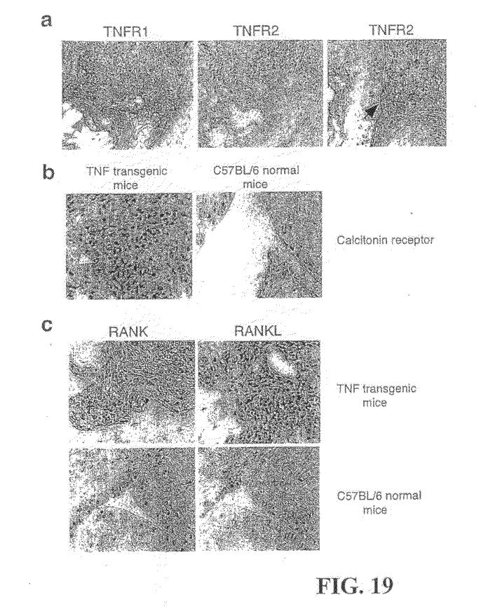

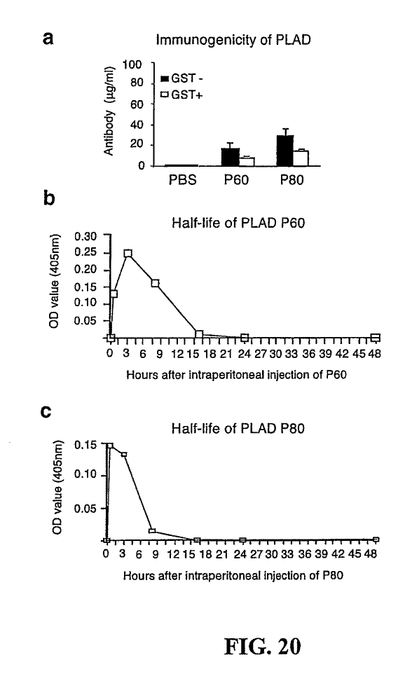

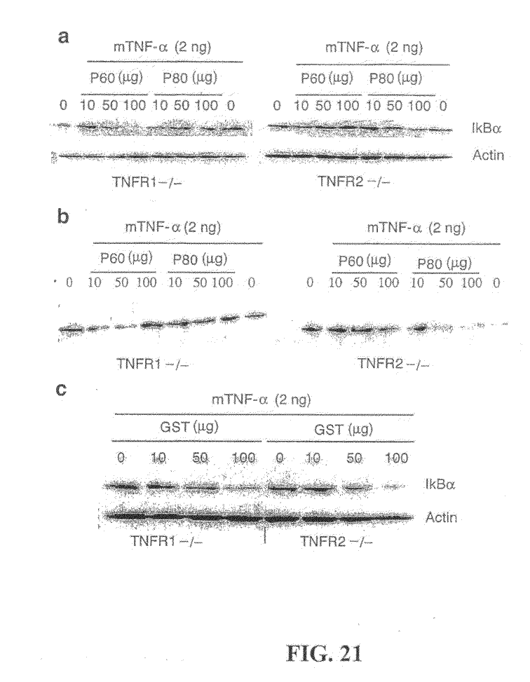

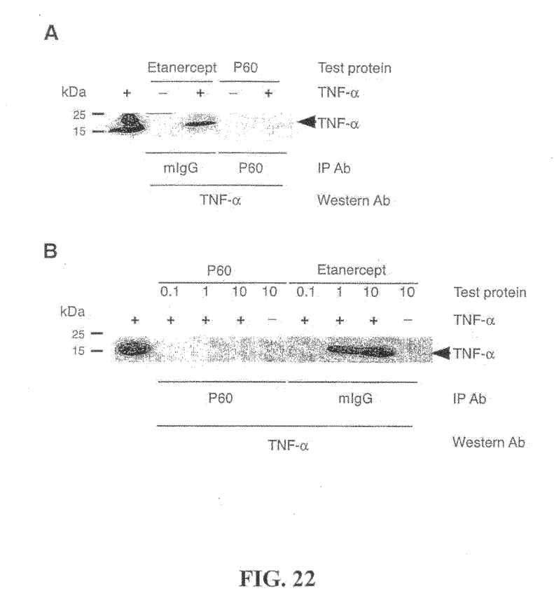

The importance of the N-terminal portion of p80 (a.a.10-54) is further illustrated by appending it to the p60 receptor which then interacted with full-length p80 (FIG. 1D, compare lane 3 to lanes 4 and 5, FIG. 9). Thus, this domain was sufficient to mediate specific association of a heterologous receptor. This association is ligand-independent because the chimera p80₁₀₋₅₄60₅₅₋₂₁₁(R1) has two amino acids encoded by an EcoRI restriction site inserted at the junction of the p80 and p60 sequences that abolished TNFα binding (FIG. 1E, panels k and l). Thus, a novel functional domain distinct from the ligand-binding pocket of the TNFR-ECD mediates self-assembly in the absence of ligand. Henceforth, this domain is referred to as the Pre-Ligand Assembly Domain (PLAD).

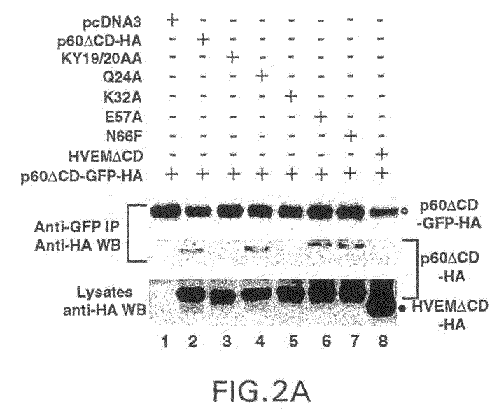

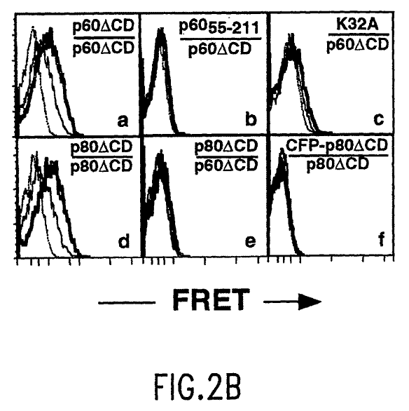

The deletion of the PLAD from either p60 or p80 completely abrogated ligand binding (Table 2 and FIG. 1E, compare panels d, f, and h). However, addition of the PLAD from p80 enabled the PLAD-deleted p60 to bind TNFα but, as indicated above, this was abolished by the two amino acid insertion encoded by an EcoRI site (FIG. 1E, panels j and 1). Thus, efficient TNFα binding by TNFRs could depend on receptor self-assembly. Alternatively, removal of the PLAD may have disrupted the overall ECD structure. The latter possibility is unlikely as previous results showed that removal of CRD1 does not disrupt proper folding of the p60 ECD (13). To definitively distinguish these possibilities, however, amino acid substitutions into CRD1-3 of p60ΔCD-HA were introduced and their effect on interaction with p60ΔCD-GFP-HA was determined. Mutagenesis was performed using the Quikchange method (Stratagene) as per manufacturer's instructions. The mutations were confirmed by DNA sequencing. All amino acids replaced except K19 are conserved between p60 and p80. The corresponding residue in p80 is E22. Two substitutions in the PLAD that are not expected to disturb direct ligand contact, KY19/20AA and K32A (5), abrogated self-association (FIG. 2A, compare lanes 3 and 5 to lane 2) and eliminated TNFα binding (Table 2). Substitution of another residue within the PLAD, Q24A, did not affect self-association or TNFα binding (FIG. 2A, lane 4 and Table 2). The residue numbers referred to in Table 2 are based on the mature sequence of p60. Two other substitutions outside of the PLAD in the CRD2 ligand binding pocket, E57A and N66F, disrupted TNFαt binding but had little effect on receptor self-association (Table 2 and FIG. 2A, lanes 6 and 7). It was also found that the association of a mutant receptor lacking the cytoplasmic tail with the wild type ECD correlates with its ability to dominantly interfere with p60-induced apoptosis, indicating that the mutant receptors enter into endogenous functional p60 receptor complexes via the PLAD (Table 2). These results show that the PLAD is physically distinct from the ligand contact domain but is nonetheless essential for efficient TNFα binding and receptor function.

Unlike monomeric receptor chains, the cytoplasmic portions of the receptor chains within a pre-assembled receptor complex might be expected to be in close proximity to each other. TNFα binding could then cause fighter association of the cytoplasmic domains leading to the recruitment of signaling proteins. To evaluate this hypothesis, a novel flow cytometric approach described in Example II (11) was employed to analyze fluorescence resonance energy transfer (FRET) between two spectral variants of GFP, cyan fluorescent protein (CFP) as the fluorescence donor and yellow fluorescent protein (YFP) as the fluorescence acceptor (12). FRET is a powerful approach to measure molecular interactions in living cells. Since energy transfer is rapidly attenuated as the distance between fluorophores increases, FRET between GFP variants allows the detection of molecular interactions within 100 Å.

Chimeric proteins were generated in which the cytoplasmic regions of p60 and p80 were replaced by either CFP or YFP and tested to determine if energy transfer occurs, between different receptor pairs. FRET was performed with a dual laser FACSvantage machine that excites the YFP protein at 514 nm prior to exciting the CFP protein at 413 nm. Energy transfer from CFP to YFP was then detected as emission at 546 nm. Cells were transfected with a large excess of YFP protein compared with CFP protein. FRET was then analyzed on the CFP positive populations using the program Flowjo (Treestar Inc.).

Energy transfer between p60ΔCD-CFP and p60ΔCD-YFP which increased substantially following the addition of TNFα (FIG. 2B, panel a) was observed. This FRET was abolished by deletion of the PLAD or by the K32A mutation that prevented PLAD association (FIG. 2B, panels b and c). Similar analyses of the p80ΔCD-CFP-p80ΔCD-YFP pair revealed a strong FRET signal that again increased with TNFα addition (FIG. 2B, panel d). Controls using p60ΔCD-YFP as acceptor for p80ΔCD-CFP or CFP-p80ΔCD (CFP fused to N-terminus of the extracellular domain) as donor showed no FRET (FIG. 2B, panels e and f). Together, these data demonstrate in living cells that the p60 and p80 chains are in close proximity to themselves and that ligand induces a change in the complexes that leads to fighter association of the CFP and YFP moieties in the cytoplasm.

Comparison of the PLAD of p60 and p80 with the first CRD of a number of receptors in the TNFR family reveals conservation beyond the cysteines that form the disulfide bond scaffold of the domain (FIG. 3A, Y23, P36, G37 and T50 in p80). Certain other TNFR-like receptors including DR4 and Fas show less conservation in the CRD 1, raising the question of whether a PLAD-like domain exists in these receptors. Whether or not ligand-independent self-association occurs in other members of the TNFR superfamily was explored.

Strikingly, the ECDs of TRAIL receptor 1 (DR4) and CD40 both self-associate but do not significantly interact with ECDs from other TNFR-like receptors (FIG. 3B). These chimeras also showed homo-specific FRET (FIG. 3C). As described in Example II, Fas (CD95/APO-1) also specifically associates with itself (11). Thus, self-assembly through the PLAD is a conserved feature of the TNFR superfamily.

This invention reveals that the p60 and p80 TNFRs pre-assemble into functional complexes in the absence of ligand via a novel N-terminal domain termed PLAD. This reveals how CRD1 plays a crucial role in ligand-binding and receptor signaling for p60 and p80 (10). Until now, the fundamental concept of signaling by members of the TNFR superfamily is that ligand brings monomer receptor chains into apposition in three-fold complexes which leads to recruitment of cytoplasmic signal transduction proteins (1, 3, 5, 6). This model was based largely upon the crystal structure of p60 complexed with ligand, which showed that three receptor chains embrace the trimeric ligand in its intersubunit grooves and remain at least 40 Å apart. The ligand makes contact with the elongated CRD2 and CRD3 domains whereas the CRD1 domains do not interact with ligand or with each other (5). The recent description of the structure of DR5/TRAIL complex reveals similar receptor-ligand interactions (13). However, the liganded structure does not appear to reflect the receptor structure prior to ligand binding. It is now clear that p60 and p80 self-associate on the cell surface and are only found as monomers if the PLAD is deleted. Cross-linking the endogenous p60 and p80 receptors suggests that trimers are a favored conformation, but other oligomeric complexes may also occur.

How ligand interacts with the pre-associated receptor complex is of great interest since it is now evident that pre-association is required for TNFα binding. TNFR signaling could be explained by one of two broad classes of models: 1) chain rotation and rearrangement, and 2) supercluster formation models (FIG. 3D). In chain rotation and rearrangement model, ligand intercalates into the pre-formed receptor trimer, causing disruption of the PLAD contacts as well as rotation and realignment of the chains into a trimer stabilized exclusively by contacts with the ligand trimer. Alternatively, ligand binding may trigger the clustering of pre-assembled TNFR trimers in which the PLAD contacts are not fully disrupted. The presence of PLAD-mediated pre-assembled TNFR trimers sheds new light on important aspects of signaling by this large family of receptors, many of which are known to be critical for lymphocyte function and homeostasis (2). Specific homotypic ECD contacts and conservation of key residues in the PLAD are characteristic of members of the TNFR superfamily including receptors that signal through death domains (p60, DR4 and Fas) and those that do not (p80 and CD40). The pre-sorting of chains into homotypic complexes on the cell surface could promote the efficiency and specificity of response. “Receptor interference” in which, for example, a p80 chain (lacking a death domain) is recruited by TNFα into a complex with p60 and causes dominant inhibition of apoptosis would be avoided. The fact that p80 actually enhances p60-induced apoptosis by providing an independent pro-apopototic signal supports this notion (15). Pre-formed trimers may also circumvent the requirement to sequentially recruit receptor chains to “build” a complex as might be required by the conventional model, thus accounting for the rapid signaling achieved through TNFR-like receptors (3).

Pre-assembly has been described for other receptor families, notably IL-1 and IL-2, which are comprised of heteromers of different polypeptides (16). Of particular interest is a recent description of pre-association of the erythropoietin receptor dimers that apparently undergo a “scissors-type” movement to accommodate ligand (17). In that case, self-association of the receptor chains occurs via the same amino acid contacts that are critical for ligand binding (17). By contrast, the T superfamily utilizes a dedicated self-association domain distinct from the CRD2/3 ligand contact region. Identification of the PLAD could allow new treatments of diseases caused by TNFα or related ligands through the use of therapeutics that specifically inhibit the pre-ligand assembly of TNFR-like receptors and thereby prevent signaling.

### Example II

Heterozygous mutations encoding abnormal forms of Fas (CD95/APO-1) dominantly interfere with Fas-induced lymphocyte apoptosis in the human Autoimmune Lymphoproliferative Syndrome (ALPS). This invention demonstrates that, rather than depending on ligand-induced receptor oligomerization, this stems from pre-association of wild-type and mutant Fas receptors through the extracellular domain Pre-associated Fas receptor complexes were found to be essential for signal transduction, and were demonstrated in living cells using a novel application of FRET between variants of the Green Fluorescent Protein (GFP). These results provide a new molecular mechanism for Fas signaling and dominant interference in human disease.

Fas (APO-1/CD95) is a cell surface receptor that transduces apoptotic signals critical for immune homeostasis and tolerance (19-21). Fas is a 317 amino-acid type 1 membrane glycoprotein with three extracellular cysteine-rich domains (CRD) that are characteristic of the tumor necrosis factor receptor (TINE) superfamily.

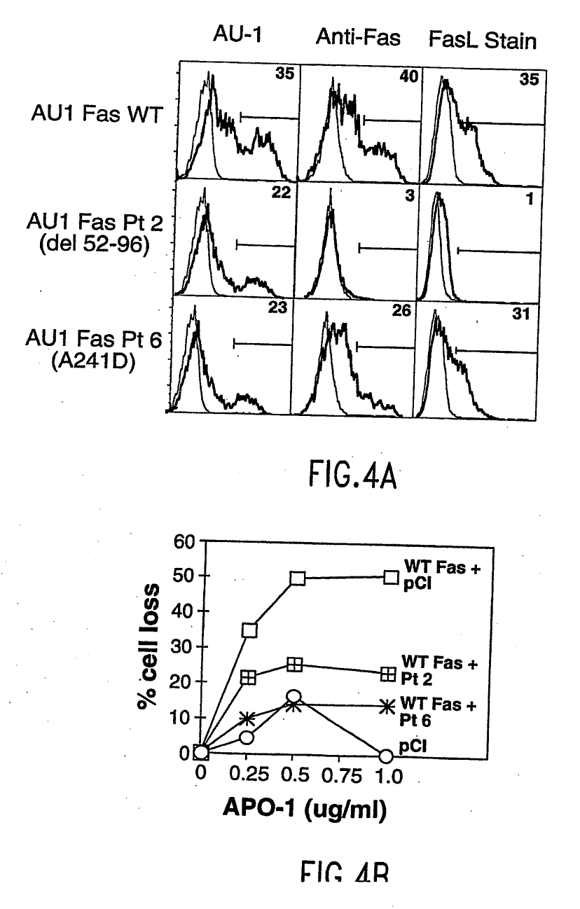

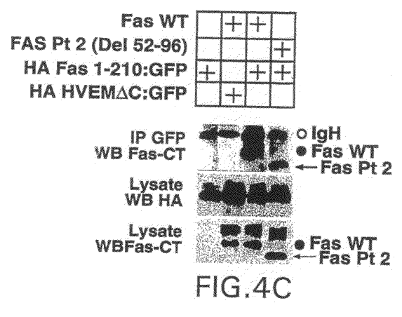

In humans, lymphocytes from patients with ALPS Type 1A harboring heterozygous Fas mutations have reduced Fas-induced apoptosis, and transfection of the mutant allele causes dominant interference with apoptosis induced through Fas (29-34). This was thought to be due to ligand-mediated cross-linking of wild-type and defective Fas chains into mixed trimer complexes that cannot recruit downstream signaling molecules. However, a dominant-interfering mutation that causes an extracellular domain (ECD) deletion of most of CRD 2 (Pt 2, deletion a.a. 52-96) through altered RNA splicing has been studied. Expression of this mutant on Fas-negative 293T cells shows no binding to agonistic antibodies (FIG. 4A) (33,35). This mutant also failed to bind to trimerized FasL, while ALPS mutations in the cytoplasmic death domain, e.g. Pt 6, A241D did not affect FasL binding or APO-1 binding (FIG. 4A). Even without binding agonistic antibodies or FasL, the Pt 2 mutant dominantly interfered with Fas-induced apoptosis almost as efficiently as the Pt 6 death domain mutant (FIG. 4B). Surface staining of co-transfected cells showed no reduction in Fas expression compared to those transfected with WT Fas alone, ruling out the possibility that the mutant Fas molecules inhibited expression of the normal allele (36). Thus dominant interference cannot be explained by the conventional model of signaling by FasL-induced crosslinking of receptor monomers, because in this scheme, the Pt 2 mutant Fas molecule would not become part of a mixed receptor complex. Therefore, ligand-independent interactions between Pt 2 Fas and wild-type Fas were tested using constructs in which the cytoplasmic domain of wild-type Fas was replaced with the Green Fluorescent Protein (GFP) (HA-Fas 1-210:GFP) to avoid spurious interactions through the death domain (24,37). Both full-length and the Pt 2 Fas receptor co-precipitated with the Fas 1-210:GFP chimera in the absence of FasL (FIG. 4C). This interaction was specific, since another member of the TNFR family, the Herpesvius Entry Mediator, fused to GFP (HVEMΔCD:GFP) did not immunoprecipitate Fas.

In order to conduct these immunoprecipitation studies, 293T cells were transfected with Fugene 6 (Boehringer Mannheim) according to the manufacturer's instructions. Cells were lysed in 150 mM NaCl, 20 mM Tris.Cl [pH 7.5], 1 mM EDTA, 5 mM iodoacetamide, 2 mM dithiothreitol (DTT), 10% glycerol, 1% TRITON X-100 and protease inhibitors Boehringer Mannheim). After pre-clearing with protein G agarose beads (Boehringer Mannheim) and normal mouse IgG, proteins were immunoprecipitated with 1 mg anti-GFP] (Roche Molecular Biochemicals) and protein G agarose beads. Immune complexes were washed three times with lysis buffer. AU1 was immunoprecipitated with 2 μl of anti-AU1 (Covance) and protein A beads. Proteins were electropheresed on Tris/Glycine gels (Novex), transferred to nitrocellulose membranes, and blotted with the indicated antibodies. Bands were visualized with SuperSignal WestDura (Pierce). Densitometry was performed with 1D image analysis software (Kodak).

In Example I, a conserved N-terminal domain, termed the “pre-ligand assembly domain” (PLAD) is described that mediates specific self-association of other members of the TNFR superfamily. However, the N-terminus of Fas is lacking several key amino-acids conserved in other TNFR-family receptors, raising the issue of whether Fas contains a functional PLAD.

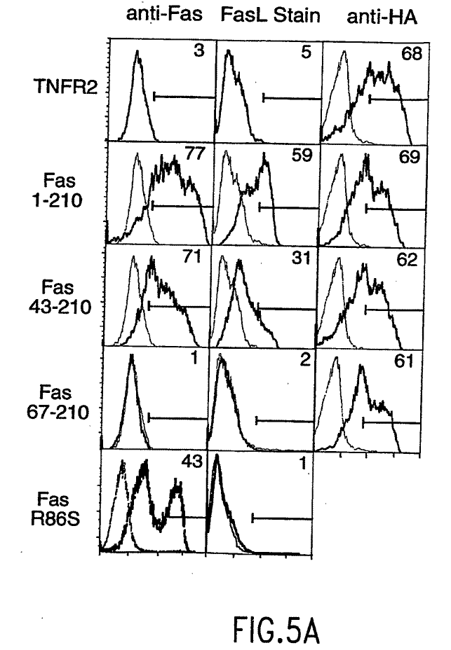

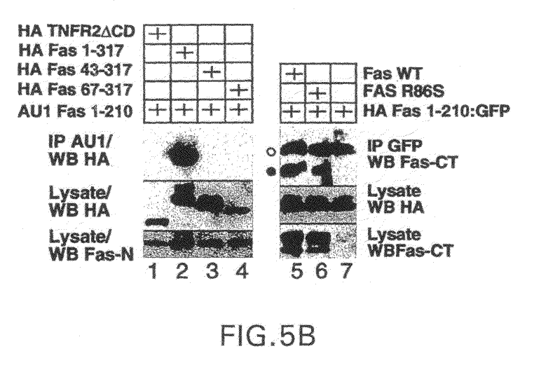

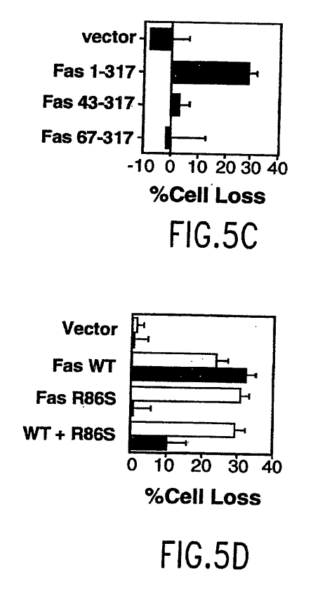

N-terminal Fas mutants truncating or eliminating the first CRD were constructed and tested for ligand binding, Fas-Fas association, and apoptotic function (FIG. 5). Fas truncation mutants were created by PCR mutagenesis with appropriate primers and Pwo high fidelity polymerase (Roche Molecular Biochemicals). For the AU-1 tagged receptors, a template with an AU-1 tag previously inserted into the region upstream of Fas CRD1 was used. For HA tagging, mutations were cloned into the EcoRI/XhoI sites of a modified pcDNA3 vector containing the leader sequence of p80 followed by an HA tag sequence. Point mutations were created with the Quickchange technique (Stratagene), substituting Pwo for Pfu polymerase. Mutations were verified by restriction enzyme mapping and automated sequencing.

These studies indicated that deleting the first 43 amino acids (a.a.) of the mature Fas protein that make up the first CRD subdomain (39) substantially reduced ligand binding but did not prevent binding of the APO-1 agonist antibody. Deleting the first 66 a.a. encoding the entire CRD1 abrogated binding of both FasL and APO-1 (FIG. 5A). Both of these deletions showed corresponding defects in apoptosis initiated by the APO-1 antibody (FIG. 5C), as well as a loss of co-precipitation of the truncated chains with a differentially tagged Fas 1-210 protein containing the complete ECD (FIG. 5B, lanes 14). Thus, despite partial FasL binding and normal APO-1 binding, removal of as little as 43 a.a. from the N-terminus of Fas prevented apoptosis induction, correlating with the loss of association of these truncated receptors with wild-type Fas. The loss of FasL binding by the 66 a.a. deletion (comprising CRD1) was surprising in light of the fact that most predicted contacts with FasL are found in CRD2 and CRD3 (22,23). Comparing these results with those obtained with the p60 and p80 TNFRs in Example I, it was hypothesized that ligand-independent pro-assembly of Fas receptor complexes may be critical to allow efficient FasL binding and receptor signaling. To further explore the requirement for ligand binding in receptor self-association, a Fas point mutation, R86S, that removes a crucial CRD2 contact residue for FasL was tested (23) and does not bind FasL when expressed on the cell surface (FIG. 5A, bottom panels). The overall receptor structure was preserved, as indicated by staining by two different agonistic anti-Fas antibodies (FIG. 5A and (18)), and self-association with intact Fas still occurred as shown by co-immunoprecipitation (FIG. 5B, lanes 5-7). Even more significantly, when co-expressed with the wild-type (WT) receptor, this mutant dominantly interfered with FasL-induced apoptosis without itself binding FasL (FIG. 5D, filled bars). Apoptosis induced with the APO-1 antibody in the same cells was unimpaired in all transfections indicating that both receptors were functionally expressed on the cell surface (FIG. 5D, open bars). Thus, dominant interference is independent of ligand binding by both naturally occurring and engineered Fas mutants. Instead, Fas function correlates with the ability to self-associate.

To quantitate Fas receptor self-association in living cells, flow cytometric and microscopic assays based on fluorescence resonance energy transfer (FRET) between spectrally distinct mutants of GFP, Cyan fluorescent protein (CFP) and Yellow fluorescent protein (YFP) were developed. CFP and YFP have spectral properties favorable for FRET in that the emission maximum for CFP is near the absorption maximum for YFP (40). Since FRET between these proteins rapidly declines at distances larger than 50 Å-100 Å, the presence of FRET between CFP and YFP fusion proteins indicates close proximity of their fluorescent protein domains. When Fas receptors with C-terminal in-frame fusions to CFP and YFP (at position 210 in place of the death domain) were co-transfected into 293T HEK cells, they were appropriately expressed on the cell surface (36).

In-frame CFP and YFP fusions with Fas and other TNF family receptors were generated by standard PCR cloning techniques and correct protein expression was confirmed by western blotting and fluorescence microscopy. 293T cells were transfected with 1 μg of the indicated YFP fusion protein constructs and 2 μg of the indicated CFP constructs. 24-36 hours later cells were harvested in PBS and analyzed on a FACSvantage cytometer with a krypton laser (Spectrophysics) tuned to 413 nm for CFP and an ILT air-cooled laser tuned to 514 nm for YFP. CFP was detected with a 470 nm/20 nm bandpass filter. YFP and FRET were detected with 546 nm/10 nm bandpass filters with signals from the 514 and 413 nm lasers respectively. Cells were sequentially illuminated with the 514 and 413 nm lasers so that all three signals could be detected from each cell. Compensation was applied so that there was no FRET signal visible from cells transfected with CFP or YFP alone. 50,000 events were collected from each sample and the data was analyzed by the FlowJo software package (Treestar) For FRET efficiency measurements, CFP emission intensities from co-transfected cells were measured on a fluorescence microscope before and after bleaching the YFP with 5 min illumination through a 505-545 nm bandpass filter. Controls showed that this much intensity bleached YFP essentially completely, with very little direct bleaching of CFP. Such direct bleaching was corrected for. FRET efficiencies were calculated using the formula

E%=[1−(CFP emission before YFP-bleach/CFP emission after YFP bleach)]*100%.

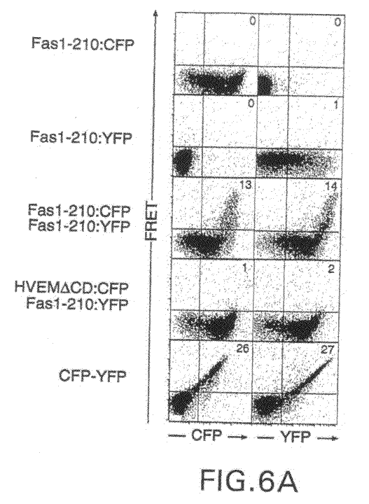

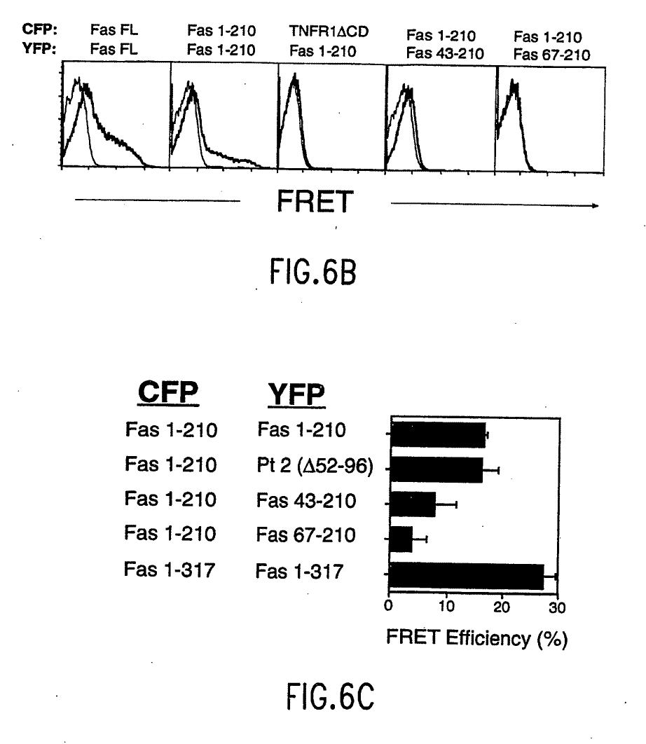

When examined by flow cytometry, CFP excitation of cells co-transfected with the CFP and YFP Fas fusion proteins triggered strong fluorescence emission at the YFP wavelength attributable to FRET (FIG. 6A, Fas 1-210:CFP/Fas 1-210:YFP), especially at high levels of YFP expression. As a positive control, a construct in which CFP was covalently fused to YFP through a 9 a.a. peptide linker (CFP-YFP) was utilized (41). In these cells, a strong FRET signal was also detected that increased linearly with expression levels. FRET was detected between Fas fusion proteins with or without the death domain, but not between Fas and the TNF family members TNFR1 or HVEM (FIGS. 6A and B). The N-terminal truncated versions of Fas that truncate or remove the PLAD gave reduced FRET signal when co-expressed with Fas 1-210 (FIG. 6B). To quantify the FRET efficiency between these different receptor mutants, microscope-based measurements of CFP dequenching after selectively photobleaching the YFP acceptor molecule, which is another characteristic of FRET (40),24) (FIG. 6C) were made. Association of Fas lacking the death domain with itself resulted in FRET with an observed efficiency of 16%. With the death domain on both molecules, FRET efficiency rose to 27%, indicative of the oligomerization property of the death domain (42). Pt 2 Fas gave a comparable FRET efficiency to Fas 1-210 indicating nearly normal self-association, but there was reduced signal with Fas 43-210 and no significant FRET efficiency with Fas 67-210. These results suggest that Fas molecules specifically self-associate on the cell surface and that this property is dependent on the PLAD.

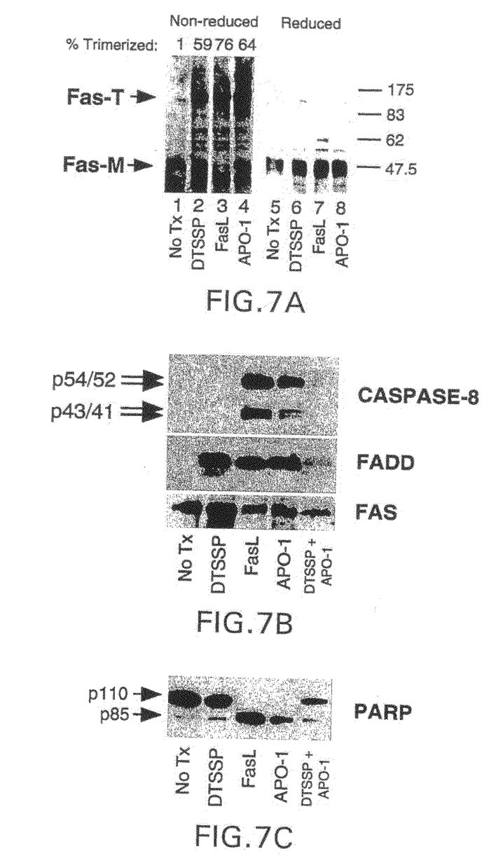

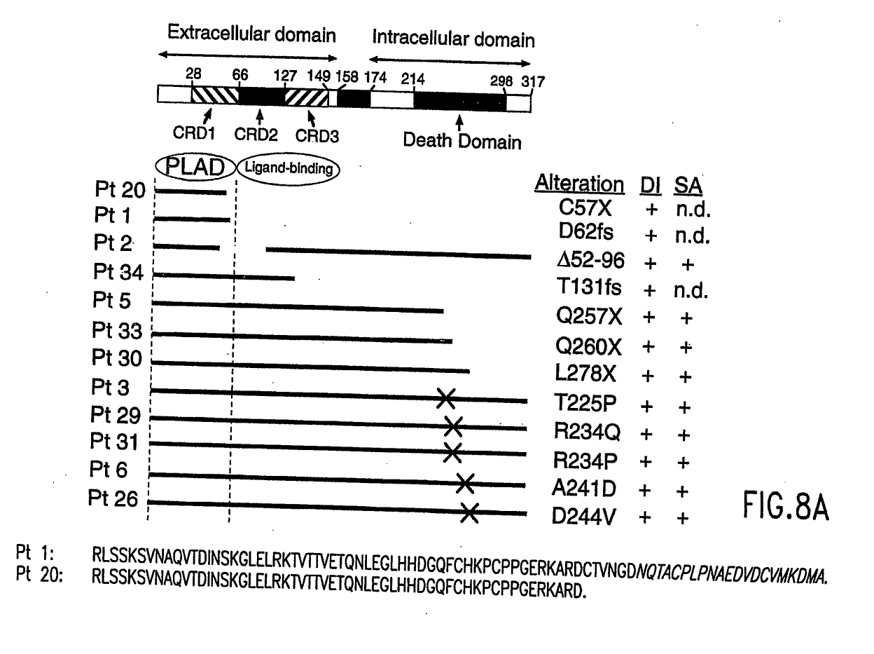

To test whether native Fas receptors self-associate on the surface of untransfected T lymphocytes, chemical cross-linking studies (FIG. 7) were performed. Addition of the cell-impermeant thiol-cleavable crosslinker 3,3′-dithiobis[sulfosuccinimidyl propionate] (DTSSP) shifted the apparent molecular weight of Fas in deglycosylated cell lysates from 45 to 140 kD, corresponding to the formation of Fas trimers (FIG. 7A, lane 2). Densitometric comparison with the monomer bands suggested that 60% of the Fas chains were cross-linked as trimers. Cleavage of the crosslinker with dithiothreitol (DTT) reduced most of these trimeric complexes to a unit state (FIG. 7A, lanes 5-8). DTSSP-induced complexes were similar to those found after stimulation with the APO-1 agonistic antibody or FasL without chemical crosslinking (FIG. 7A, lanes 3-4) (25). Agonist-induced complexes were linked by intermolecular disulfide bonds, shown by reduction with DTT (FIG. 7A, lanes 7-8). Examination of immunoprecipitated Fas signaling complexes from these cells showed that antibody or ligand stimulation triggered recruitment of FADD and caspase-8 and led to proteolysis of caspase-8 into its 41 and 43 kD processed forms (FIG. 4B) as well as the caspase-dependent cleavage of poly(ADP-ribose)polymerase (PARP) (FIG. 7C). However, signaling complexes in cells treated with DTSSP showed moderate FADD association but no caspase-8 binding or processing and no PARP cleavage, indicating that chemical crosslinking of the pre-associated receptor complex is not sufficient to trigger apoptotic signaling. Interestingly, pre-treatment with DTSSP prevented the formation of active signaling complexes in response to subsequent APO-1 treatment (FIG. 7B). These results show that non-covalent preassociation of Fas receptors is not dependent on overexpression. Ligand binding triggers a change in the structure of the receptor complex associated with interchain disulfide bond formation and intracellular signaling. Chemical crosslinking of Fas receptors appears to capture pre-associated complexes in a non-signaling state.

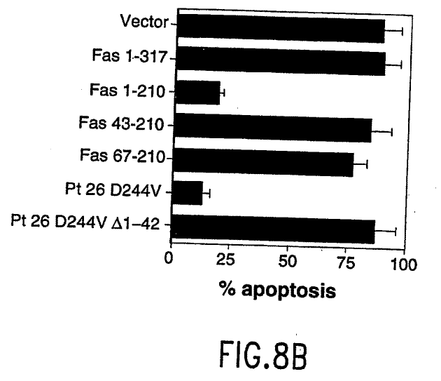

The conserved N-terminal PLAD was required for appropriate Fas receptor function, and could thus play a key role in dominant interference in ALPS. Comparing the structure, dominant interference (DI), and Fas-Fas self-association (SA) of a large number of ALPS patients that have been studied at the National Institutes of Health (29, 33-35) (FIG. 8), it was found that the PLAD was preserved in every example of a dominant-interfering mutation associated with disease, including mutations that affect either the extracellular or intracellular portions of Fas. In Pts 1 and 20, mutations create premature termination polypeptides encoding only the first 57 and 62 a.a. of the mature Fas protein, suggesting that the PLAD itself was sufficient for dominant interference (FIG. 8A). Removal of all or part of the death domain (Pts 5, 30 or 33) or abrogating its FADD binding function by point mutations (Pts 3, 6, 26, 29, or 31) create potent dominant-interfering Fas molecules (29). Therefore, whether the PLAD was required for dominant interference by an engineered termination mutant of Fas that eliminates the death domain (Fas 1-210) was tested. Results indicated that both N-terminal truncations abolished dominant interference by Fas 1-210 (FIG. 8B). Truncation of the PLAD (deleting up to ala 42) in a Fas death domain point mutant from an ALPS patient (ALPS Pt 26, D244V) eliminated the dominant-inhibitory effect of this natural mutant (FIG. 8B).

Together these findings redefine the mechanism by which Fas mutations in ALPS dominantly interfere with normal Fas function. It is now evident that dominant-interfering Fas mutations preserve the N-terminal PLAD because this domain is responsible for complex formation between wild-type and mutant Fas molecules. The central molecular principle of genetic dominant interference is that mutant proteins must physically interact with wild-type proteins in a specific functional complex (43). Previously, dominant negative receptor mutations associated with human diseases have been shown to interfere with normal receptor signaling by sequestering ligand, blocking intracellular signaling or preventing transport of the WT chain to the cell surface (44). For Fas, the data show that dominant interference stems from a novel mechanism involving PLAD-mediated association between wild type and mutant receptors prior to ligand binding. These findings explain why the abnormal Fas protein in ALPS Pt 2 and other Fas ECD mutants can fail to bind FasL and yet exert dominant interference sufficient to cause disease. PLAD-mediated interactions also account for the dominant-interfering interactions of the large number of ALPS patients that carry mutations affecting the death domain of Fas, since removing the PLAD abrogated the dominant negative function of Fas molecules with deleted or mutated death domains. PLAD interactions are also likely involved in the down-modulation of Fas-induced apoptosis by soluble alternatively spliced forms of Fas that all include this domain (45). Natural receptor mutants that do not encode a functional PLAD would not be expected to be dominant-interfering. PLAD-mediated dominant interference may also play a role in modulation of signaling by decoy receptors (20) and in the pathogenesis of diseases due to heterozygous genetic abnormalities in other members of the TNFR family.

These results also suggest a new model for understanding transmembrane signaling by Fas, involving conversion of pre-associated trimers to a signaling state by ligand, rather than ligand-induced oligomerization of individual receptor chains. The FET studies allow estimation of the distance between CFP and YFP-tagged Fas molecules on the cell surface in the absence of ligand. The Förster radius, R₀, for randomly oriented CFP and YFP is 50 Å (23). Assuming that CFP and YFP in the fusions to Fas are equally expressed, randomly oriented with respect to each other, and randomly assorted into equilateral trimers, the observed FRET efficiencies (FIG. 3C) suggest an upper limit of 57 Å for the distance between CFP and YFP chromophores fused to full-length Fas molecules and 65 Å for fusions to Fas 1-210. These distances are much closer than what would be observed for randomly distributed molecules on the cell surface, and were specific, since FRET was not observed between Fas and other TNFR-family receptors. The fact that FRET required a threshold level of YFP expression (FIG. 3A, FAS1-210:CFP/FAS 1-210:YFP) could reflect the statistics of mixing CFP-labeled and YFP-labeled Fas, or an actual dependence on receptor density for preassociation. Since pre-association enhances Fas signaling, regulating the amount of receptor pre-association through changes in Fas expression or other means is a novel mechanism for modulating apoptosis signaling.

Signaling through receptor complex rearrangement may be a widely-used mechanism to ensure rapid and specific cellular responses to ligands. However, this signaling mechanism also confers susceptibility to dominant interference by naturally occurring receptor variants or pathogenic heterozygous mutations in ALPS.

### Example III

### Methods

Mice and reagents. BALB/c, C57BL/6, DBA/1J, C3H/HeJ, C3H/HeN, TNFR1 and TNFR2 knockout mice were purchased from the Jackson Laboratory. TNF-transgenic mice were purchased from Taconic. Male mice at 6-8 weeks of age were used in all experiments and were housed in the animal facility of the Laboratory of Immunology, NIAID, NIH. CpG DNA was synthesized by CBER. CpG DNA 1668 sequence is 5′-TCCATGACGTTCCTGATGCT-3′ (SEQ ID NO:61) (50). Mouse monoclonal antibodies (MAbs) against P60 (1H11) or P80 PLAD (3H11) were prepared by using P60 PLAD and P80 PLAD.
Purification of PLAD-GST fusion protein. Cloning and purification of PLAD-GST fusion protein was carried out according to an established procedure (78). Purified PLAD-GST fusion proteins were validated on a 4-20% Tris-Glycine gel and by mass spectroscopy and stored at 80° C. LPS was removed with Detoxi-Gel AffinityPak Columns (Pierce).
In vitro tests of P60 and P80 PLAD proteins. L929 cells were collected after trypsin treatment. Cells were pretreated with different doses of PLAD protein for 30 min, after which TNF-α (2 ng) was added. 42.3 cells pretreated similarly with PLAD protein or GST. Following the 30 minute' pretreatment, TNF-α (3 ng), antibody to Fas (11) (10 ng), and protein A (20 ng) were added and incubated at 37° C. Quantitation of cell death was performed by hemocytometer or flow cytometry at indicated time-points. TNF-α binding was measured according to the manufacturer's protocol (R&D systems).
Immunogenicity and half-life of PLAD proteins. Immunogenicity in vivo and half-life of PLAD proteins by use of ELISA were investigated. Both PLAD proteins could elicit an antibody response at least half of which was directed at the GST portion (Supplementary FIG. 5a). The in vivo half-lives of P60, or P80 PLAD protein were measured by ELISA on blood taken at various times and were approximately 5 h (Supplementary FIG. 5b, c).
Development of arthritis triggered by TNF-αe, CpG DNA with PLAD protein treatment. TNF-α, CpG DNA or LPS were intramuscularly injected with or without 100 μg of PLAD protein into mouse knee joints. The mice were sacrificed and joints were removed for histopathological examination 3 d after injection.
Induction and treatment of CIA with PLAD protein. Chicken collagen type II (Sigma) emulsified with an equal volume of complete Freund's adjuvant (Sigma) was intradermally injected with 0.1 ml of emulsion containing 100 μg of type II collagen at the base of the tail of male DBA/1J mice. Mice were boosted with 100 μg of type II collagen emulsified with an equal volume of incomplete Freund's adjuvant (Sigma) at 21 d. Preventive treatment with PLAD protein or PBS began at 22 d after primary immunization and was administered intraperitoneally three times per week until day 52 in a coded fashion. Therapeutic treatment with P60 PLAD protein or PBS began three d after mice have already developed arthritis. P60 PLAD protein (400 μg administered every other day) was given intraperitoneally for two weeks and then the treatment was switched between groups. In the “masked” experiments, mice were analyzed by two independent examiners unaware of the treatment every third day and assessed for extent of arthritis: paw swelling and clinical score. Joint swelling was determined by measuring the thickness of the paws with a caliper. Clinical arthritis was evaluated using the following scale: grade 0, no swelling; grade 1, slight swelling and erythema; grade 2, pronounced swelling; grade 3, joint rigidity. Each limb was graded as score of 0-3 with a maximum possible score of 12 for each animal.
Histopathological examination of joints. After routine fixation, decalcification and paraffin embedding of the tissue, joint sections were cut and stained with hematoxylin and eosin. All the slides were coded and evaluated blindly by researchers. The extent of synovitis, pannus formation, or bone/cartilage destruction was judged on a scale from grade 0 (no signs of inflammation), grade 1 (mild inflammation with hyperplasia of the synovial lining layer, minimal without cartilage destruction) to grades 2 and 4 (increasing degrees of inflammatory cell infiltrate, or cartilage and bone destruction).
In vitro osteoclastogenesis assay. Bone marrow cells were extracted from the tibia of BALB/c mice and cultured in α-MEM medium in presence of M-CSF. After 3 d, bone marrow macrophages were induced further for 4 d with TNF-α (20 ng/ml) and M-CSF (50 ng/ml) in the presence or absence of PLAD proteins. Cells were then fixed and stained for the osteoclast marker TRAP using the manufacturer's procedure (Sigma).
Immunofluorescence (66). Mononuclear cells isolated from spleen of TNFR1 or TNFR2 knockout mice were treated with TNF-α with or without P60 and P80 PLAD protein in vitro. Fixed and permeabilized cells were stained with Rabbit antibody to NF-κB p65 (C-20, Santa Cruz). Cells were then washed and incubated with a FITC-conjugated donkey anti-rabbit antibody. Nuclear NF-κB was analyzed and scored in masked fashion on a confocal microscope.
Statistical analysis. The difference of incidence and severity in groups was analyzed by use of the Fisher's test and Mann-Whitney U test, respectively. P≦0.05 was considered to be significant.

### Results

### Recombinant PLAD-GST Fusion Protein

To obtain soluble proteins containing the PLAD domain of the human P60 or P80 TNFRs, glutathione-S-transferase (GST) fusion proteins were prepared. Affinity purification provided P60 and P80 PLAD-GST fusion proteins whose full-length molecule weights were 34 kD and 33 kD, respectively, the expected sizes based on amino acid sequences (FIG. 1b and supplementary FIG. 1). Western blots and protein sequencing by mass spectroscopy were carried out to confirm that both bands in the gel are P60 or P80 PLAD proteins (FIG. 1c).

### PLAD Protein Inhibits TNF-α-Induced Cell Death

This experiment was performed to determine whether the purified PLAD proteins inhibit the cytopathic effect of TNF-α on L929 cells (63). The purified P60 PLAD protein clearly inhibited mouse and human TNF-α-induced cell death in a dose-dependent fashion (FIG. 2a, b). The protection was comparable to infliximab and etanercept (Supplementary FIG. 2). By contrast, a control CD40 PLAD protein had no effect (FIG. 2c). Human 42.3 cells that require signals from both P60 and P80 to undergo death induced by TNF-α (15) were also tested. It was found that either the P60 or the P80 PLAD protein inhibited TNF-α- but not anti-Fas-induced cell death (FIG. 2d). A GST alone protein does not inhibit TNF-α- or anti-Fas-induced cell death. Also, the P60 PLAD protein inhibited TNF-α-induced caspase-8 activation in L929 cells as effectively as etanercept or infliximab (FIG. 2e). Thus, purified human P60 or P80 PLAD proteins efficiently blocked either human or mouse TNF-α.

### P60 PLAD Inhibits TNF-α or CpG DNA-Induced Arthritis

Whether PLAD proteins could inhibit TNF-α-induced arthritis in vivo was also tested. Arthritis was induced by intra-articular injection of mouse TNF-α (45 ng) with or without 100 μg PLAD protein added. The P60 PLAD protein powerfully inhibited the features of TNF-α-induced arthritis including synovitis, pannus, and bone erosion (FIG. 3a, b). By contrast, the P80 PLAD or the GST protein alone did not significantly affect disease (FIG. 3a, c) (Supplementary FIG. 3a).

TNF-α plays an important role in the pathogenesis of SA (49, 50, 61). CpG-containing bacterial DNA and LPS are bacterial components that strongly induce TNF-α release from monocytes and macrophages that can trigger arthritis (49, 61). Co-injection of 100 μg P60 PLAD protein could significantly reduce pannus and bone destruction in CpG DNA-induced arthritis (P<0.05) (FIG. 3a, d), but 100 μg P80 PLAD did not (Supplementary FIG. 3b). The P60 PLAD protein, but not the P80 PLAD protein, could significantly reduce LPS-induced arthritis.

### P60 PLAD Inhibits MMP Expression in Collagen-Induced Arthritis (CIA)

Matrix metalloproteinases (MMP) are important mediators of destruction of cartilage and bones during arthritis, and MMP expression can be induced by TNFα. Therefore, this experiment addressed whether P60-PLAD protein inhibited MW expression in CIA. The results showed that P60 PLAD significantly inhibited MMP expression in CIA rig. 25). Note that the dark (brown in its original color slide) staining in the left panel indicates M expression.

P60 PLAD Inhibits iNOS Expression in Collagen-Induced Arthritis (CIA)

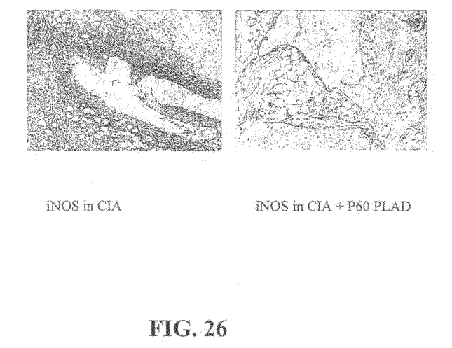

Nitric oxide (NO) is a short-lived free radical gas. Excessive nitric oxide is toxic and causes chronic inflammation. Importantly, it has been shown that TNFα can stimulate macrophages/monocytes to produce nitric oxide by activating inducible nitric oxide synthase (iNOS) and increase NO level, thus causing local joint damage. Therefore, it was determined whether P60-PLAD protein inhibits iNOS expression in CIA. The results showed that P60-PLAD protein markedly inhibited iNOS expression in affected joints of CIA (FIG. 26). Note that the dark (brown in its original color slide) staining in the left panel indicates iNOS expression. These results support the teaching herein that by interfering TNFR assembly, P60 PLAD blocks TNFα-induced tissue damage in CIA.

### CONCLUSION

The present results demonstrate that the PLAD in TNFR1 is an important therapeutic, target. CD95 PLAD was found to participate in pathogenesis of ALPS by mediating dominant interference (11). The P60 and P80 PLAD proteins block binding of TNF-α to TNFR1 but do not directly bind to TNF-α. Most importantly, the P60 PLAD exhibited a striking ameliorative effect in several mouse models of arthritis through systemic as well as intra-articular administration. Moreover, inflammatory cell infiltration of the joints was markedly reduced by P60 PLAD treatment. In previous examples, it was found that the PLADs for P60 and P80 are selective for homotypic binding and do not cross-bind with each other or the CD95 PLAD (11, 53). Those experiments demonstrated that P60 and P80 PLAD had preferential effects on the corresponding receptors but might not be completely specific (FIG. 5e, f). Furthermore, the P60 PLAD can arrest key aspects of the disease process in several mouse models of arthritis. Taken together, our data shows that the PLAD is an important drug target for arthritis.

The P60 PLAD protein potently arrested NF-κB induction by TNF-α. The P60 and

P80 PLAD proteins can inhibit TNF-α-induced NF-κB translocation in a receptor preferential manner. Moreover, NF-κB is required for RANKL-induced osteoclastogenesis (71). Osteoclast-mediated bone and cartilage erosion causes most of the permanent damage in arthritis (67, 68, 69). The present results show osteoclast activation in pannus and sites of bone destruction in TNF-α transgenic mice. Also, there was abundant expression of RANK and RANKL in arthritic joint of the TNF-α transgenic mice and collagen-induced arthritis (69). P60 PLAD treatment dramatically reduced RANK and RANKL in the lining layer and pannus.

Infliximab and etanercept both can directly bind and block the effects of TNF-α and they are now widely used in the treatment of RA and other diseases (48). However, side effects such as lupus-like disease, mycobacterial infections, and an increased incidence of lymphoma have been observed (72-75). Both drugs directly block TNF-o binding to both TNFRs. Thus, they could inhibit potentially beneficial effects mediated by TNF2 while arresting the disease-causing effects of TNFR1. The P60 PLAD is small protein domain that could preferentially target TNFR1. In our models, a dose of P60 PLAD protein of about 4 mg/kg (intra-articularly) or 16 mg/kg (parenterally) had similar effects to doses of infliximab (10 mg/kg) (76) and etanercept (0.4 mg/kg)³³ that have been used clinically in the amelioration of arthritis. The P60 PLAD protein offers a new approach to inhibit the pathogenic effects of TNF-α that is useful in RA.

### Example IV

### PLAD Protein Directly Binds to TNF Receptor

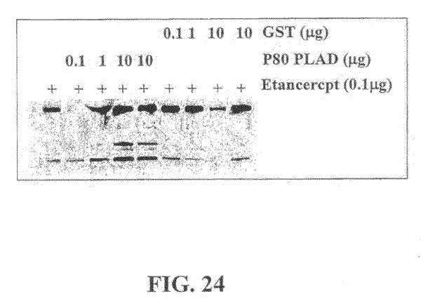

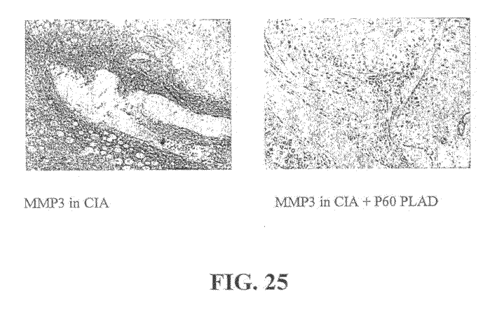

To determine whether the PLAD protein directly binds to TNFR, P80-PLAD protein was mixed with etanercept, a TNFR2 fusion protein that contains complete extracellular domain of TNFR2, was tested by mixing with P80 PLAD protein. The results of immunoprecipitation (IP) and Western Blot (WB) show that P80-PLAD-GST protein but not the GST-only proteins can associate with etanercept, indicating that PLAD protein directly binds to TNFR and therefore inhibit TNFα activity (FIG. 24).

TABLE 1

| | | | | Accession | | | | |
|---|---|---|---|---|---|---|---|---|
| | | | | (human Locus | Human | Mouse | | |
| TNFR family Receptor | | Atlases | | Link) | Chromosome | Chromosome | | Ligands |
| NGFR | TNFRSF16 | p75 | | M14764 | 17q21-q22 | 11 | 55.8 cM | NGF |
| | | | | (4804) | | | | |
| Troy | TNFRSF19 | Tajos | | AF167655 | 13q12.11-12.3 | 14 | | ? |
| | | | | (55504) | | | | |
| EDAR | | | | AF130888 | 2q11-q13 | 10 | 29.0 cM | EDA-A1 |
| | | | | (19808) | | | | |
| XEDAR | | EDA-A2R | | AF298812 | X | | | EDA-A2 |
| CD40 | TNFRSF5 | p50 | 8p50 | X60592 | 20q12-q13.2 | 2 | 97.0 cM | CD40 Ligand |
| | | | | (958) | | | | |
| DcR3 | TNFRSF6B | | | AF104419 | 20q13 | | | Fas Ligand |
| | | | | (8771) | | | | |
| FAS | TNFRSF6 | CD95 | APO-1 | M87454 | 10q24.1 | 19 | 23.0 cM | Fas Ligand |
| | | APT1 | | (14102) | | | | |
| HvaA | TNFRSF14 | HVEM | ATAR | U70321 | 1p36.3-p38.2 | | | LIGHT |
| | | TR2 | LIGHTR | (8764) | | | | |
| OX40 | TNFRSF4 | CD134 | ACT35 | X75362 | 1p36 | 4 | 79.4 cM | OX40 Ligand |
| | | TXGP1L | | (7293) | | | | |
| AITR | TNFRSF18 | GITR | | AP125304 | 1p36.3 | 4 | | AITR Ligand |
| | | | | (8784) | | | | |
| CD30 | TNFRSF8 | Ki-1 | D1S158E | M88854 | 1p38 | 4 | 75.5 cM | CD30 Ligand |
| | | | | (948) | | | | |
| 4-1BB | TNFRSF8 | CD137 | ILA | L12954 | 1p35 | 4 | 76.5 cM | 4-1BB Ligand |
| | | | | (3604) | | | | |
| TNFR2 | TNFRSF1B | CD120b | p75 | M32315 | 1p38.3-p38.2 | 4 | 75.5 cM | TNF |
| | | TNFBR | p80 | (7183) | | | | Lymphotoxin |
| | | TNF-R-II | TNFR2 | | | | | |
| DR3 | TNFRSF12 | TRAMP | WSL-1 | U72783 | 1p35.2 | | | ? |
| | | LARD | WSL-LR | (8716) | | | | |
| | | DDR3 | TR3 | | | | | |
| | | APO-3 | | | | | | |
| CD27 | TNFRSF7 | Tp55 | S152 | M53928 | 12p13 | 6 | 60.35 cM | CD27 Ligand |
| | | | | (939) | | | | |
| TNFR1 | TNFRSF1A | CD120S | p55-R | M75888 | 12p13.2 | 6 | 60.55 cM | TNF |
| | | TNFAR | TNFR1 | (7132) | | | | Lymphotoxin |
| | | p80 | TNF-R-1 | | | | | |
| LTBR | TNFRSF3 | CD | TNFR2-RP | LD4270 | 12p13 | 6 | 60.4 cM | TNF |
| | | TNFCR | | (4055) | | | | Lymphotoxin |
| | | TNF-R-III | | | | | | LIGHT |
| RANK | TNFRSF-11A | TRANCER | | AF018253 | 18q22.1 | | | RANK Ligand |
| TAC1 | | CAML interactor | | AF023814 | 17p11 | 11 | | Blys |
| | | | | (23495) | | | | April |
| BCMA | TNFRSF17 | BCM | | Z29574 | 16p13.1 | | | Blys |
| | | | | (606) | | | | April |
| DR8 | | TR7 | | NM_014452 | 8p21.1-12.2 | | | ? |
| | | | | (27242) | | | | |
| OPG | TNFRSF11B | oatcoprotegerin | TR1 | U94332 | 8q24 | | | RANK Ligand |
| | | OCIF | | (4982) | | | | TRAIL |
| DR4 | TNFRSF10A | Apo2, | TRAILR-1 | U80875 | 8p21 | | | TRAIL |
| | | | | (8797) | | | | |
| DR5 | TNFRSF10B | KILLER | TRICK2A | AF012828 | 8p22-p21 | | | TRAIL |
| | | TRAIL-R2 | TRICKB | (8795) | | | | |
| DcR1 | TNFRSF10C | TRAILR3 | LIT | AF012538 | 8p22-p21 | | | TRAIL |
| | | TRID | | (8794) | | | | |
| DcR2 | TNFRSF10D | TRUNDD | TRAILR4 | AF029761 | 8p21 | | | TRAIL |
| | | | | (8783) | | | | |

TABLE 2

| | | CLONE | TNFα | SELF | DOMINANT |
|---|---|---|---|---|---|
| | MAB225* | 4.12† | BINDING‡ | ASSOCIATION§ | INTERFERENCE\|\| |
| p60ΔCD | 1 | 1 | 1 | + | + |
| p60₅₅₋₂₁₁ | 0.87 | 0.02 | 0.01 | NT | − |
| K19E | 1.05 | 1.07 | 1.1 | + | + |
| KY19/20AA | 0.59 | 0.14 | 0.03 | − | − |
| Q24A | 1.06 | 0.97 | 1.13 | + | + |
| K32A | 0.36 | 0.01 | 0.01 | − | − |
| DT49/50AA | 1.16 | 0.96 | 1.13 | NT | + |
| E57A | 1.68 | 0.04 | 0.02 | + | + |
| T61A | 1.42 | 1.28 | 1.35 | NT | + |
| N66F | 0.67 | 0.04 | 0.01 | + | + |
| R77A | 1.06 | 0.99 | 1.24 | NT | + |
| W108T | 1.32 | 1.11 | 1.25 | NT | + |
| L112E | 1.31 | 1.27 | 0.85 | NT | + |

*Staining of p60-specific monoclonal antibody clone MAB225 (R&D Systems). The values were normalized against the staining of HA epitope tag by dividing the pencentage of MAB225 positive cells with the percentage of HA positive cells.
†Staining with p60-specifc monoclonal antibody clone 4.12 (Zymed) was normalized against HA staining.
‡TNFα binding was determined by using a biotinylated from of TNFα and normalized against HA staining (25).
§Self-association was determined by immunoprecipitation assays in 293T transient transfections as described (12).
NT, not tested.
||Dominant interference was determined as described (26). Dominant inhibition by the p60ΔCD-HA mutants was at least 50% of p60ΔCD wild type (+).
P60₅₅₋₂₁₁, KY19/20AA and K32A did not confer any protection (<5%) against TNF-induced death (−) in all experiments.
The antibodies and TNFα binding to p60ΔCD is arbitrarily set at 1. Results are representative of three independent experiments.

TABLE 1
Members of the TNF/NFR superfamily

| | | | | |
|---|---|---|---|---|
| | | | Human | Mouse |
| Standardized | Other Names | Accession | Chromosome | Chromosome |

Receptor

| | | | | | |
|---|---|---|---|---|---|
| NGFR | TNFRSF16 | p75 | M14764 | 17q21-q22 | 11, 56.8 cM |
| Troy | TNFRSF16 | Taj | AF187555 | 13q12.11-12.2 14 | |
| EDAR | | | AF130998 | 2q11-q12 | 10, 28.0 cM |
| XEDAR | EDA-A2R | | AF288312 | X | |
| CD4D | TNFRSF6 | p60, 8p60 | XB0592 | 20q12-q13.2 | 2, 97.0 cM |
| DcF3 | TNFRSF88 | | AF104419 | 20q12 | |
| FAB | TNFRSF8 | CO85, APO-4, APT1 | M67454 | 10q24.1 | 19, 23.0 cM |
| OXAD | TNFRSF4 | CO134, AOT25, TXGPIL | X75962 | 1p36 | 4, 79.4 cM |
| AnR | TNFRSF19 | GITA | AF125304 | 1p282 | 4 |
| CD20 | TNFRSF9 | 1q-1, D15188E | MB3554 | 1p36.3 | 4, 75.5 cM |
| HvBA | TNFRSF14 | HVEM, ATAR, TR2, | U70221 | 1p38.2-p38.2 | |
| | | LIGHTR | | | |
| 4-1B8 | TNFRSF9 | CO127, ILA | L12954 | 1p38 | 4, 76.5 cM |
| TNFR2 | TNFRSF18 | CO12 [symbol] , p75, TNFBR, | M22315 | 1p38.2-p38.2 | 4, 76.5 cM |
| | | TNFRBD, TNF-R-11 | | | |
| OR3 | TNFR [symbol] F12 | TRAMP, WEL-t, LARD, | U72763 | 1p38.2 | |
| | | [symbol] SL-LR, DDR3, TR3, | | | |
| | | APQ- [symbol] | | | |
| CD27 | TNFRSF7 | Tp55, S152 | M [symbol] | 12p13 | 8, 60.35 cM |
| TNFR1 | TNFRSF1A | CO1208, P55-R, TNFAR | M75868 | 12p13.2 | 8, 8055 cM |
| | | TNFRED TNF-R-1 | | | |
| LTBR | TNFRSF3 | TNFR2-RP, TNFCR, | L04270 | 12p19 | 8, 80.4 cM |
| | | TNF-R-m | | | |
| RANK | TNFRSF11A | TRANCE-R | AF018253 | 18q22.1 | |
| TACI | CA [symbol] IL | | | AF023614 | 17p11 |
| | interactor | | | | |
| BCMA | TNFRSF17 | BCM | Z28574 | 16p13.1 | |
| DR6 | TR7 | HM014452 | 8p21.1-12.2 | | |
| OPG | TNFRSF11B | CCIF, TR1 | U94332 | 9Q24 | |
| | | ostaoprotegerin | | | |
| DR4 | TNFRSF10A | Apo2, TRAILR-1 | U90975 | 9p21 | |
| DR5 | TNFRSF10B | KILLER, TRICK2A, | AF012629 | 9p22-p21 | |
| | | TRAIL-R2, TRICKB | | | |
| DcR1 | TNFRSF10C | TRAILR3, LIT, TRID | AF012538 | 9p22-p21 | |
| DoR2 | TNFRSF10D | TRUNDD TRAILR4 | AF029781 | 9p21 | |

Ligand

| | | | | | |
|---|---|---|---|---|---|
| EDA | | EDA1 | NM_001289 | Xq12-q13.1 | X, 37.0 cM |
| CD40L | TNFRF8 | IMDA, HIGM1, TRAP, | XB7978 | Xq28 | X, 19.0 cM |
| | | CD154, EP³⁹ | | | |
| FasL | TNFSF8 | APTILG1 | U11921 | 1q23 | 1, 95.0 cM |
| OX40L | TNFSF4 | Sp34 TXGP1 | D90224 | 1q25 | 1, 91.9 cM |
| AITRL | TNFSF18 | TL6, hGITRL | AF125303 | 1q23 | |
| CD39L | TNFSF8 | | L08753 | 9q23 | 4, 92.2 cM |
| VEGI | TNFSF15 | TL1 | AF039390 | | |
| LIGHT | TNRSF14 | LT_HVEM-L | AF038591 | 19 (probable) | 17 |
| 4-1BBL | TNFRF9 | | U02298 | 19p12.3 | 17 |
| CD27L | TNFSF7 | CD70 | L09096 | 19p13 | 17, 20.0 cM |
| LTα | TNFSF1 | TNFS, LT | X01293 | 6p21.3 | 17, 19.08 cM |
| TNF | TNFSF2 | tumor necrosis factor, | X01394 | 6p21.2 | 17, 19.06 |
| | | cachectin, TNFA, DIF | | | |
| LTβ | TNFSF3 | TNFO, p33 | L11015 | 6p21.3 | 17,19.061 |
| TNEAK | TNFSF12 | DR3L APO3L | AF030099 | 17p13 | 117 |
| APRIL | TNFSF13 | | NM_003909 | 17p13.1 | 117 |
| BLYD | TNFSF19B | BAFF, THANK, TALL1 | AF132600 | 12q32-34 | |
| RANKL | TNFSF11 | TRANCE, OPGL, DDF | AF013171 | 13q13 | 14, 45.0 |
| TRAIL | TNFSF10 | Apo-2L TL2 | U37512 | 3q28 | |

| | |
|---|---|
| Phenotypes associated | Additional functional |
| with mutations | observations |

Receptor

| | | |
|---|---|---|
| NGFR | Defective sensory nauron in- | |
| | nervation impaired neat | |
| | sensitivity | |
| Troy | | Expressed in hairfollicles and |
| | | epithelium; the mouse gene |
| | | is located near the urated |
| | | coat locus. |
| EDAR | Hypohydrotic ectodermal dys- | |
| | plasta - abnormal tooth, hair | |
| | and sweet gland formation | |
| XEDAR | Likely role in skin, hair and | |
| | | tooth formation |
| CD4D | Defective Ig class switching | |
| | and Go formation causing | |
| | immunodeficiency | |
| DcF3 | | Secreted decay receptor for |
| | | Fast with possible role in |
| | | tumor erosion |
| FAB | impaired activation-induced T | |
| | cell death lymphoproliferation; | |
| | autoimmunity (ALPO) | |
| OXAD | Defective T cell responses | |
| AnR | | Glucocorticoid-induced; |
| | | inhibits T cell receptor- |
| | | dependent epoptosis |
| CD20 | | Matter of Fleed-Sterrbarg cells |
| | | in [symbol] disease |
| HvBA | | Probable role in T cell proliferation |
| | | and receptor for herpes |
| | | simplex virus |
| [symbol] | Probable role in T cell | |
| | | responses |
| TNFR2 | Increased senstivity to bacterial | |
| | pathogens; decreased | |
| | sensitivity to LPS; reduced | |
| | antigen-induced T cell | |
| | apoptosis | |
| OR3 | | A linked, partially duplicated |
| | | copy of the gene encodes a |
| | | potential decay receptor |
| CD27 | Defective T cell responses | |
| TNFR1 | Impaired clearance of bacterial | |
| | pathogens; resistance to | |
| | LPS; LH present; defective | |
| | GO formation; defective PP | |
| | formation | |
| LTBR | Absence of LH, PP, defective | |
| | GC formation | |
| RANK | Osteopetrosis; absence of osteoctasis; | Required for lactating mammary |
| | absence of lymph nodes; | gland development |
| | PP present; abnormal | |
| | B cell development | |
| TACI | 11 | Probable role in B cell responses |
| BCMA | | Probable role in B cell responses |
| DR6 | | |
| OPG | Osteoporosis, arterial calcification | |
| DR4 | | Probable inducer of tymphocyte |
| | | death and activation |
| DR5 | | Probable inducer of tymphocyte |
| | | death and activation |
| DcR1 | | GPI-linked decoy receptor- |
| | | interferes with TRAIL |
| | | signaling |
| DoR2 | | Transmembrane decoy receptor- |
| | | interferes with TRAIL |
| | | signaling |

Ligand

| | | |
|---|---|---|
| EDA | Hypotrydrotic ectoderned | |
| | dysptasia-abnormal tooth, | |
| | hair and sweet grand | |
| | formation | |
| CD40L | Detactive T cell and IgG responses; | |
| | tryper IgM syndrome | |
| FasL | Impartred activation-induced | |
| | T cell death, lymphopromeration, | |
| | autotraomuritty, ALPS | |
| OX40L | Defactive T cell responses | |
| AITRL | | Inhibits T cell receptor-dependerit |
| | | apoptosts |
| CD39L | | Possible role in malignant |
| | | tymptrocyte disorders |
| VEGI | | Potertial vascular endotreted |
| | | cell growth inhibitor |
| LIGHT | | |
| 4-1BBL | Defective T cell responses | |
| CD27L | | |
| LTα | Absence of LN and PP, ctsorgarized | |
| | spienic microarchitecture; | |
| | defective GO formation | |
| TNF | LN present, defective-GO formation, | |
| | increased susceptibility | |
| | to microbial pathogans | |
| LTβ | Absence of peripheral LN and | |
| | PP, presence of measenteric | |
| | and same cerital LN, defective | |
| | GO formation | |
| TNEAK | | Potential role in monocyte and |
| | | NK cell cytotoxicity |
| APRIL | | Probable role in B cell responses |
| BLYD | | Probable role in B cell responses |
| RANKL | Ostaopatrosis, absence of ostaociasts; | Required for lactating mammary |
| | absence of lymphnodes; | grand development |
| | PP; present, normal | |
| | spleric architacture; abnormal | |
| | B cell and T cell development | |
| TRAIL | | |

[symbol]

indicates data missing or illegible when filed

Throughout this application, various publications are referenced. The disclosures of these publications in their entireties are hereby incorporated by reference into this application in order to more fully describe the state of the art to which this invention pertains.

### REFERENCES

1. K. J. Tracey and A. Cerami, Annu. Rev. Cell Bio. 9, 317 (1993); K. J. Tracey and A. Cerami, Annu. Rev. Med. 45, 491 (1994).
2. H. Wajant, K. Pfeffer, K. Pfizenmaier and P. Scheurich, Cytokine & Growth Factor Reviews 9, 297 (1998); A. Ashkenazi and V. M. Dixit, Science 281, 1305 (1998); G. G. Klaus, et al., Int. Rev. Immunol. 15, 5 (1997); R. Horie and T. Watanabe, Semin. Immunol. 10, 457 (1998).
3. M. Lenardo, et al., Annu. Rev. Immunol. 17, 221 (1999); D. Wallach, et al., Annu. Rev. Immunol. 17, 331 (1999); A. M. Chinnaiyan, K. O'Rourke, M. Tewari, et al., Cell 81, 505 (1995); M. P. Boldin, et al., J. Biol. Chem. 270, 7795 (1995); M. Muzio, et al., Cell 85, 817 (1996); M. P. Boldin, T. M. Goncharov, Y. V. Goltsev, et al., Cell 85, 805 (1996); H. Hsu, J. Huang, H. Shu, et al., Immunity 4, 387 (1996); H. Shu, M. Takeuchi, D. V. Goeddel, Proc. Natl. Acad. Sci. U.S.A. 93, 13973 (1996); A. T. Ting, F. X. Pimentel-Muinos, B. Seed, EMBO J. 15, 6189 (1996).
4. Y. Jiang, J. D. Woronicz, W. Liu, et al., Science 283, 543 (1999).
5. D. Banner, et al., Cell 73, 431 (1993).
6. L. Tartaglia, D. Goeddel, J. Biol. Chem. 267, 4304 (1992); H. Loetscher, et al., J. Biol. Chem. 266, 18324 (1991); C. A. Smith, T. Farrah, R. G. Goodwin, Cell 76, 959 (1994).
7. S. J. Jones, et al., J. Immunol. 162, 1042 (1999).
8. M. Boldin, et al., J. Biol. Chem. 270, 387 (1995).
9. L. Zheng and M. J. Lenardo, unpublished data
10. S. A. Marster, A. D. Frutkin, N. J. Simpson, et al., J. Biol. Chem. 267, 5747 (1992); K. C. Hsu and M. V. Chao, ibid. 268, 16430 (1993); P. C. Chen, G. C. DuBois, M. Chen, ibid. 270, 2874 (1995); T. Reid, P. Louie, R. A. Heller, Circ. Shock 44, 84 (1994); A. E. Corcoran, et al., Eur. J. Biochem. 223, 831 (1994).
11. Siegel R. M. Frederiksen, J. K., Zacharias, D. A. Chan, D. A., Johnson, M., Lynch, D., Tsien, R. Y. & Lenardo. M. J. Fas preassociation required for apoptosis signaling and dominant inhibition by pathogenic mutations. Science 288, 2354-2357 (2000).
12. L. Tron, et al., Biophys. J. 45, 939 (1984); J. Szollosi, et al., Cytometry 5, 210 (1984); R. Y. Tsien, Annu. Rev. Biochem. 67, 509 (1998).
13. S. G. Hymowitz, et al., Molecular Cell 4, 563 (1999); J. Mongkolsapaya, et al., Nature Structural Biology 6, 1048 (1999).
14. J. Naismith, T. Devine, B. J. Brandhuber, et al., J. Biol. Chem. 270, 13303 (1995); J. Naismith, T. Devine, T. Kohno, et al., Structure 4, 1251 (1996); J. Naismith, B. Brandhuber, T. Devine, et al, J. Mol. Recog. 9, 113 (1995).
15. Chan, F. K. & Lenardo M. J. A crucial role for p80 TNF-R2 in amplifying p60 TNF-R1 apoptosis signals in T lymphocytes. Eur. J. Immunol. 30, 652-660 (2000); R. A. Heller, K. Song, N. Fan, et al., Cell 70, 47 (1992); T. Weiss, et al, J. Immunol. 158, 2398 (1997); V. Haridas, et al., ibid. 160, 3152 (1998); W. Declercq, et al, ibid. 161, 390 (1998).
16. C. Guo, S. K. Dower, D. Howlowka, B. Baird, J. Biol. Chem. 270, 27562 (1995); S. Damjanovich et al., Proc. Natl. Acad. Sci. U.S.A. 93, 13973 (1996); T. Gadella Jr. and T. M. Jovin, J. Cell. Biol. 129, 1543 (1995).
17. O. Livnah et al., Science 283, 987 (1999); I. Remy, I. A. Wilson, and S. W. Michnick, Science 283, 990 (1999).
18. A. R. Schievella, J. H. Chen, J. R. Graham, et al, J. Biol. Chem. 272, 12069 (1997).
19. D. Wallach, et al., Annu. Rev. Immunol 17, 331 (1999).
20. A. Ashkenazi, V. M. Dixit, Science 281, 1305 (1998).
21. M. Lenardo, et al., Annu. Rev. Immunol. 17, 221 (1999).
22. J. R. Orlinick, A. Vaishnaw, K. B. Elkon, M. V. Chao, J. Biol. Chem. 272, 288-89 (1997).
23. G. C. Starling, et al., J. Exp. Med. 185, 1487 (1997).
24. A. M. Chinnaiyan, K. O'Rourke, M. Tewari, V. M. Dixit, Cell 81, 505 (1995).
25. F. C. Kischkel, et al., EMBO Journal 14, 5579 (1995).
26. B. Huang, M. Eberstadt, E. T. Olejniczak, R. P. Meadows, S. W. Fesik, Nature 384, 638 (1996).
27. D. A. Martin, R. M. Siegel, L. Zheng, M. J. Lenardo, J. Biol. Chem. 273, 4345 (1998).
28. D. Nicholson, N. Thornberry, TIBS 22, 299 (1997).
29. D. A. Martin, et al., Proc. Nat. J. Acad. Sci. USA 96, 4552 (1999).
30. A. K. Vaishnaw, et al., J. Clin. Invest. 103, 355 (1999).
31. F. Rieux-Laucat, et al., Science 268, 1347 (1995).
32. J. Drappa, A. K Vaishnaw, K. E. Sullivan, J. L. Chu, K B. Elkon, N. Engl. J. Med. 335, 1643 (1996).
33. G. H. Fisher, et al., Cell 81, 935 (1995).
34. S. E. Straus, M. Sneller, M. J. Lenardo, J. M. Puck, W. Strober, Ann. Intern. Med. 130, 591 (1999).
35. C. Jackson, et al., Am. J. of Hum. Genet. 64, 1002 (1998).
36. R. M. S and M. J. L., unpublished observations.
37. M. P. Boldin, et al., J. Biol. Chem. 270, 7795 (1995).
38. F. C. Chan, et. al., accompanying manuscript (1999).
39. J. H. Naismith, S. R. Sprang, TIBS 23, 74 (1998).
40. A. Miyawaki, R. Tsien, Meth. Enzymol. In Press (2000).
41. N. P. Mahajan, D. C. Harrison-Shostak, B. Michaux, B. Herman, Chem. Biol. 6, 401 (1999).
42. M. P. Boldin, et al., J. Biol. Chem. 270, 387 (1995).
43. L Herskowitz, Nature 329, 219 (1987).
44. E. Jouanguy, et al., Nat Genet 21, 370 (1999); R. Levy-Toledano, L. H. Caro, D. Accili, S. I. Taylor, Embo J 13, 835 (1994); M. P. Cosma, M. Cardone, F. Carlomagno, V. Colantuoni, Mol. Cell. Biol. 18, 3321 (1998).
45. G. Papoff et al., J Immunol. 156, 4622 (1996).
46. W. Li, J. Schlessinger, Mol. Cell. Biol. 11, 3756 (1991); W. Li, E. R. Stanley, Embo J. 10, 277 (1991).
47. H. Walczak, et al., Nat Med 5, 157 (1999).
48. Feldmann, M. & Maini, R. N. TNF defined as a therapeutic target for rheumatoid arthritis and other autoimmune diseases. Nat. Med. 9, 1245-1250 (2003).
49. Hultgren, O. Eugster, H. P., Sedgwick, J. D., Korner, H., & Tarkowski A. TNF/lymphotoxin-alpha double-mutant mice resist septic arthritis but display increased mortality in response to Staphylococcus aureus. J. Immunol. 161, 5937-5942 (1998).
50. Deng, G. M., Nilsson, I. M. Verdrengh, M. Collins. L. V. & Tarkowski. A. Intra-articularly localized bacterial DNA containing CpG motifs induces arthritis. Nat. Med. 5, 702-705 (1999).
51. Gutierrez-Ramos. J. C. & Bluethmann. H. Molecules and mechanisms operating in septic shock: lessons from knockout mice. Immunol. Today 18, 329-334 (1997).
52. Locksley, R. M., Killeen, N., & Lenardo. M. J. The TNF and TNF receptor superfamilies: integrating mammalian biology. Cell 104, 487-501 (2001).
53. Chan, F. K., Chun, H. J., Zheng. L., Siegel. R. M., Bui, K. L., & Lenardo, M. J. A domain in TNF receptors that mediates ligand-independent receptor assembly and signaling. Science 288, 2351-2354 (2000).
54. Siegel, R. M., Chan, F. K., Chun, H. J., & Lenardo, M. J. The multifaceted role of Fas signaling in immune cell homeostasis and autoimmunity. Nat. Immunol. 1, 469-474 (2000).
55. Chan, F. K. The pre-ligand binding assembly domain: a potential target of inhibition of tumour necrosis factor receptor function. Ann. Rheum. Dis. 59 (Suppl 1):i50-3 (2000).
56. Feldmann, M., Brennan, F. M., & Maini, R. N. Role of cytokines in rheumatoid arthritis. Annu. Rev. Immunol. 14, 397-440 (1996).
57. Miossec, P. Cytokine abnormalities in inflammatory arthritis. Baillieres Clin. Rheumatol. 6, 373-392 (1992).
58. Pettipher, E. R., Higgs, G. A., & Henderson, B. Interleukin 1 induces leukocyte infiltration and cartilage proteoglycan degradation in the synovial joint. Proc. Natl. Acad. Sci. USA. 83, 8749-8753 (1986).
59. Henderson, B., & Pettipher, E. R. Arthritogenic actions of recombinant IL-1 and tumour necrosis factor alpha in the rabbit: evidence for synergistic interactions between cytokines in vivo. Clin. Exp. Immunol. 75, 306-310 (1989).
60. Nakashima, T., Wada, T., & Penninger, J. M. RANKL and RANK as novel therapeutic targets for arthritis. Curr. Opin. Rheumatol. 15, 280-287 (2003).
61. Deng, G. M., Verdrengh, M., Liu Z. Q., & Tarkowski, A. The major role of macrophages and their product tumor necrosis factor alpha in the induction of arthritis triggered by bacterial DNA containing CpG motifs. Arthritis Rheum. 43, 2283-2289 (2000).
62. Williams, R. O., Feldmann, M., & Maini, R. N. Anti-tumor necrosis factor ameliorates joint disease in murine collagen-induced arthritis. Proc. Natl. Acad. Sci. USA. 89, 9784-9788 (1992).
63. Sheehan, K. C. Pinckard, J. K., Arthur. C. D., Dehner, L. P., Goedde,1 D. V. & Schreiber, R. D. Monoclonal antibodies specific for murine p55 and p75 tumor necrosis factor receptors: identification of a novel in vivo role for p75. J. Exp. Med. 181, 607-617 (1995).
64. Keffer, J. Probert, L., Cazlaris, H., Georgopoulos, S., Kaslaris, E., Kioussis, D., & Kollias, G. Transgenic mice expressing human tumour necrosis factor: a predictive genetic model of arthritis. EMBO J. 10, 4025-4031 (1991).
65. Chen, G., & Goedde,1 D. V. TNF-R1 signaling: a beautiful pathway. Science 31, 1634-1635 (2002).
66. Su H, Bidere N, Zheng L, Cubre A, Sakai K, Dale J, Salmena L, Hakem R, Straus S, Lenardo M. Requirement for caspase-8 in NF-kappaB activation by antigen receptor. Science 307, 1465-8 (2005).
67. Redlich, K., Haver, S., Ricci R., David, J. P., Tohidast-Akrad, M., Kollias G., Steiner, G., Smolen, J. S., Wagner. E. F., & Schett, G. Osteoclasts are essential for TNF-alpha-mediated joint destruction. J. Clin, Invest. 110, 1419-1427 (2002).
68. Pondel, M. Calcitonin and calcitonin receptors: bone and beyond. Int. J. Exp. Pathol. 81, 405-422 (2000).
69. Boyle, W. J., Simonet, W. S., & Lacey, D. L. Osteoclast differentiation and activation. Nature 423, 337-342 (2003).
70. Mori, L., Iselin, S., De Libero, G., & Lesslauer, W. Attenuation of collagen-induced arthritis in 55-kDa TNF receptor type 1 (TNFR1)-IgG1-treated and TNFR1-deficient mice. J. Immunol. 157, 3178-3182 (1996).
71. Jimi, E., Aoki, K., Saito, H., D'Acquisto, F., May, M. J., Nakamura, I., Sudo, T., Kojima, T., Okamoto, F., Fukushima, H., Okabe, K., Ohya, K., & Ghosh, S. Selective inhibition of NF-kappa B blocks osteoclastogenesis and prevents inflammatory bone destruction in vivo. Nat. Med. 10, 617-624 (2004).
72. Gomez-Reino, J. J., Carmona, L., Valverde, V. R., Mola, E. M., Montero, M. D.; BIOBADASER Group. Treatment of rheumatoid arthritis with tumor necrosis factor inhibitors may predispose to significant increase in tuberculosis risk: a multicenter active-surveillance report. Arthritis Rheum. 48, 2122-2127 (2003).
73. Wolfe, F., Michaud, K., Anderson, J., & Urbansky, K. Tuberculosis infection in patients with rheumatoid arthritis and the effect of infliximab therapy. Arthritis Rheum. 50, 372-379 (2004).
74. Shakoor, N., Michalska, M., Harris, C. A., & Block, J. A. Drug-induced systemic lupus erythematosus associated with etanercept therapy. Lancet. 359, 579-580 (2002).
75. Wolfe, F., & Michaud, K, Lymphoma in rheumatoid arthritis: the effect of methotrexate and anti-tumor necrosis factor therapy in 18,572 patients. Arthritis Rheum. 50, 1740-1751 (2004).
76. Elliott, M. J., Maini, R. N., Feldmann, M., Long-Fox, A, Charles, P., et al. Repeated therapy with monoclonal antibody to tumour necrosis factor alpha (cA2) in patients with rheumatoid arthritis. Lancet 22, 1125-1127 (1994).
77. Jarvis, B., & Faulds, D. Etanercept: a review of its use in rheumatoid arthritis. Drugs 57, 945-966 (1999).
78. Zhang, Z., Jimi, E., & Bothwell A. L. Receptor activator of NF-kappaB ligand stimulates recruitment of SHP-1 to the complex containing TNFR-associated factor 6 that regulates osteoclastogenesis. J. Immunol. 171, 3620-3626 (2003).
79. Finlay, B. B., and G. McFadden. 2006. Anti-immunology: evasion of the host immune system by bacterial and viral pathogens. Cell 124:767-782.
80. Alcami, A 2003. Viral mimicry of cytokines, chemokines and their receptors. Nat Rev Immunol 3:36-50.
81. Reading, P. C., A. Khanna, and G. L. Smith. 2002. Vaccinia virus CrmE encodes a soluble and cell surface tumor necrosis factor receptor that contributes to virus virulence. Virology 292:285-298.
82. Schreiber, M., L. Sedger, and G. McFadden. 1997. Distinct domains of M-T2, the myxoma virus tumor necrosis factor (TNF) receptor homolog, mediate extracellular TNF binding and intracellular apoptosis inhibition. J Virol 71:2171-2181.
83. Papoff et al. Identification and Characterization of a Ligand-independent Oligomerization Domain in the Extracellular Region of the CD59 Death Receptor. J. Biol. Chem. 274 (53), 38241-38250 (1999).

1. A polypeptide of 38 to 125 amino acids, comprising the isolated amino acid sequence of a pre-ligand assembly domain (PLAD) of a TNF receptor-like receptor.

2. The polypeptide of claim 1, wherein the PLAD is selected from the group consisting of: the PLAD of TNF-R, the PLAD of p60, the PLAD of p80, the PLAD of Fas (CD95/APO-1), the PLAD of TRAIL, the PLAD of LTyR, the PLAD of CD40, the PLAD of CD30, the PLAD of CD27, the PLAD of HVEM, the PLAD of OX40, the PLAD of DR4, the PLAD of NGFR, the PLAD of Troy, the PLAD of EDAR, the PLAD of XEDAR, the PLAD of DcR3, the PLAD of AITR, the PLAD of 4-1BB, the PLAD of DR3, the PLAD of RANK, the PLAD of TACI, the PLAD of BCMA, the PLAD of DR6, the PLAD of OPG, the PLAD of DRS, the PLAD of DcR1, and the PLAD of DcR2.

3. A polypeptide consisting of the amino acid sequence of a pre-ligand assembly domain of a TNF receptor-like receptor.

4. A polypeptide comprising the isolated amino acid sequence of a pre-ligand assembly domain (PLAD) of a TNF receptor-like receptor, wherein the polypeptide is R₁-TNF receptor-like receptor PLAD-R₂, wherein R₁ or R₂ comprise an amino acid sequence that does not flank the TNF receptor-like receptor PLAD in a naturally occurring TNF receptor-like receptor.

5. The polypeptide of claim 1, wherein the polypeptide is R₁-TNF receptor-like receptor PLAD-R₂, wherein R₁ is H, acyl, NH₂, an amino acid or a peptide, and R₂ is H, acyl, NH₂, an amino acid or a peptide.

6. The polypeptide of claim 1, wherein the polypeptide is R₁-amino acids 1-54 of p60-R₂ (SEQ ID NO: 1), wherein R₁ is H, acyl, NH₂, an amino acid or a peptide, and R₂ is H, acyl, NH₂, an amino acid or a peptide.

7. The polypeptide of claim 1, wherein the polypeptide is R₁-amino acids 10-54 of p80-R₂ (SEQ ID NO: 2), wherein R₁ is H, acyl, NH₂, an amino acid or a peptide, and R₂ is H, acyl, NH₂, an amino acid or a peptide.

8. The polypeptide of claim 1, wherein the polypeptide is R₁-amino acids 1-43 of Fas-R₂ (SEQ ID NO: 3), wherein R₁ is H, acyl, NH₂, an amino acid or a peptide, and R₂ is H, acyl, NH₂, an amino acid or a peptide.

9. The polypeptide of claim 1, wherein the polypeptide is R₁-amino acids 1-66 of Fas-R₂ (SEQ ID NO: 4), wherein R₁ is H, acyl, NH₂, an amino acid or a peptide, and R₂ is H, acyl, NH₂, an amino acid or a peptide.

10. The polypeptide of claim 1, wherein the polypeptide is R-amino acids 13-50 of LtÿR-R₂ (SEQ ID NO:5), wherein R₁ is H, acyl, NH₂, an amino acid or a peptide, and R₂ is H, acyl, NH₂, an amino acid or a peptide.

11. The polypeptide of claim 1, wherein the polypeptide is R1-amino acids 6-39 of CD40-R2 (SEQ ID NO:6), wherein R1 is H, acyl, NH₂, an amino acid or a peptide, and R2 is H, acyl, NH₂, an amino acid or a peptide.

12. The polypeptide of claim 1, wherein the polypeptide is R₁-amino acids 11-51 of CD30-R₂ (SEQ ID NO: 7), wherein R₁ is H, acyl, NH₂, an amino acid or a peptide, and R₂ is H, acyl, NH₂, an amino acid or a peptide.

13. The polypeptide of claim 1, wherein the polypeptide is R-amino acids 7-42 of CD27-R₂ (SEQ ID NO: 8), wherein R₁ is H, acyl, NH₂, an amino acid or a peptide, and R₂ is H, acyl, NH₂, an amino acid or a peptide.

14. The polypeptide of claim 1, wherein the polypeptide is R₁-amino acids 6-37 of HVEM-R₂ (SEQ ID NO: 9), wherein R₁ is H, acyl, NH₂, an amino acid or a peptide, and R₂ is H, acyl, NH₂, an amino acid or a peptide.

15. The polypeptide of claim 1, wherein the polypeptide is R-amino acids 3-36 of OX40-R₂ (SEQ ID NO: 10), wherein R₁ is H, acyl, NH₂, an amino acid or a peptide, and R₂ is H, acyl, NH₂, an amino acid or a peptide.

16. The polypeptide of claim 1, wherein the polypeptide is R-amino acids 109-138 of DR4-R₂ (SEQ ID NO: 11), wherein R₁ is H, acyl, NH₂, an amino acid or a peptide, and R₂ is H, acyl, NH₂, an amino acid or a peptide.

17. The polypeptide of claim 3, wherein the PLAD is selected from the group consisting of: the PLAD of TNF-R, the PLAD of Fas (CD95/APO-1) and the PLAD of TRAIL.

18. An isolated nucleic acid encoding the polypeptide of claim 1.

19. The isolated nucleic acid of claim 18 in a vector.

20. The vector of claim 19 in a host suitable for expressing the nucleic acid.

21. A method of inhibiting TNF receptor oligomerization in a cell by administering an effective amount of the polypeptide of claim 1.

22. A method of inhibiting Fas oligomerization in a cell by administering an effective amount of the polypeptide of claim 1.

23. A method of inhibiting ligand binding to a TNF receptor-like receptor by administering an effective amount of the polypeptide of claim 1.

24. A method of inhibiting ligand binding to Fas by administering an effective amount of the polypeptide of claim 1.

25. A method of treating inflammation in a subject by administering an effective amount of the polypeptide of claim 1.

26. The method of claim 25, wherein the inflammation is associated with an autoimmune disorder.

27. The method of claim 25, wherein the inflammation is associated with rheumatoid arthritis, osteoarthritis or septic arthritis.

28. A composition comprising an inhibitor of PLAD association.

29. The method of claim 25, wherein the inhibitor is an antibody that specifically binds to the PLAD of a TNF receptor-like receptor.

30. The method of claim 25, wherein the inhibitor is a PLAD of a TNF receptor-like receptor.

31. The method of claim 30, wherein the soluble PLAD is PLAD of p60.

32. A method of screening for an inhibitor of PLAD-association comprising:
a) transfecting a cell with a plasmid containing a nucleic acid comprising a nucleic acid sequence encoding an isolated PLAD functionally linked to a fluorescence donor and a plasmid comprising a nucleic acid sequence encoding an isolated PLAD functionally linked to a fluorescence acceptor;
b) contacting the cell with a putative inhibitor; and
c) measuring FRET, wherein a decrease in FRET as compared to FRET measurement in a cell that was not contacted with the putative inhibitor indicates the presence of an inhibitor of PLAD-association.

33. A method of screening for an inhibitor of PLAD association comprising:
a) transfecting a cell with a plasmid containing a nucleic acid comprising a nucleic acid sequence encoding an isolated PLAD and a plasmid comprising a nucleic acid sequence encoding a second isolated PLAD;
b) contacting the cell with a putative inhibitor and;
c) measuring PLAD self association, wherein a decrease in PLAD association in the cell of step b) as compared to PLAD association in a cell that was not contacted with the putative inhibitor indicates the presence of an inhibitor of PLAD-association.
